SEQUENCE LISTING (134 sequences)

SEQ ID NO: 1 - DNA, 1353 bp, Homo sapiens
cgcctgccgttcagcctgagaggctgctgctagcctgctcacgcatgcagcccgggctgcagaggaagtgtggggaggaaggaagtgggtatagaagggtgctgagatgtgggtcttgaagagaatagccataacgtctttgtcactaaaatgttccccaggggccttcggcgagtctttttgtttggttttttgtttttaatctgtggctcttgataatttatctagtggttgcctacacctgaaaaacaagacacagtgtttaactatcaacgaaagaactggacggctccccgccgcagtcccactccccgagtttgtggctggcatttgggccacgccgggctgggcggtcacagcgaggggcgcgcagtttggggtcacacagctccgcttctaggccccaaccaccgttaaaaggggaagcccgtgccccatcaggtccgctcttgctgagcccagagccatcccgcgctctgcgggctgggaggcccgggccaggacgcgagtcctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctggcggcagaaaccgggagtggggccgggcgagtgcgcggcatcccaggccggcccgaacgctccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgtttagtgttgaatgttccccaccgagagcgcatggcttgggaagcgaggcgcgaacccggcccccgaagggccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggtgagtagagttggactgatgcgccctcagcagctcagagcggcgtgagaatggcgccctagggattccctgcccgattcaaactttcctccctcccaaggaggcaggagggaaacgctgcgagagccagctcgctggaaatctgccagggaaactaacttatcttggggcggcagcgccccgggtgcgttatggtgcaaggtcttggagcttcgagtcgggggtggtgggcggaggaggaggtggggaagctgccctgctgc

SEQ ID NO: 2 - DNA, 116 bp, Homo sapiens
cgggaccccacccagcggcccgtacccggagaagcagcgcgagcacccgaagctcccggctggcggcagaaaccgggagtggggccgggcgagtgcgcggcatcccaggccggccc

SEQ ID NO: 3 - DNA, 1374 bp, Homo sapiens
cgtcaggtatagttttgtttgggggaagaaagaaacagacccataagatccttaattaggacattattatctttttttaagttccggagtagatgtgcaggatgtgcatgtttgttacataggtaaacgtgtgctgtggtggcttgctgcacctatcaacccatcacctaagtattaagtccctctgcccccttctatcccctgcccccaggccccaatgtgtgttgttcccctccctgtgtacatgtgttctcatagaacattattttttgattcatttgtcaagttggcctgagctggtttgattaggaggtgaaatgtttatatagctgtccttggtcttcctgtccatcaatcttttccagtttgatcgcagtaaattcatttctgtaaaacttaatatctcccataaaggattcagatttcataataacagattactaaaaatgcaaatgtttagttgttactgtctttggatactttctgataaagttgagctttatcgttaatcagcttttccactgggacatggcagatatggcacagccaagccctcccggagcatagtctaatcatggcaagttttaactattgatgtaagagttttaactattgattttgctttcatcaaaagcaaagccattcacacagtatttaacaaaaaccctcgggctctgctaggttcttgtttactgttaaaacagtttaaaggcagggtcttgatcacttaaaatactcttagaagggaaagtaacattttaaaggattaacagtggaaaaagaatatccctgttcctcttccttcccccaagtcctcaacatgtgaaaaatagagttgataagtaaggagatttaaggcagaacccgaggcctacggagcagttaccttctacggcaatttcaagggtggatggtgcgagcgccgctggggcagctggcgttctggttcttactccgtgggaaaatggccctgagcccgactggcttgaggcttagacaggtgaccccgcgaagcgggtgggcaggcgcggccgaggggcgggaggcgggcagcctccgtgattggccggcggagggggcggtgtagcgacccgaggagggaagaggactccaagcgccatggccgctgccgcccgagcccgggtcgcgtacttgctgaggcaactgcaacgcgcagcgtgagtgcggggccggcgggcgggcaggggaccgcagggtgcccacgcagccccctgggtgtgacccgctcagatcctgcgcagtcttctccgcttactttcaagaattcggagaatttggataccgccctgggctcccaccgcgcgccaggcggaacagcgaggtcacagagtgcgcgatgcagcctctcagggc

SEQ ID NO: 4 - DNA, 86 bp, Homo sapiens
gcttgaggcttagacaggtgaccccgcgaagcgggtgggcaggcgcggccgaggggcgggaggcgggcagcctccgtgattggccg

SEQ ID NO: 5 - DNA, 1973 bp, Homo sapiens
cgcctgtagtgtgtcagctatgtgcattttccttcaggatgagcaaaaatatttcgtcatcctccccgctcctcaacagcgcagaagatgccgaaaagcaattacaggaccttacagagctgctccgggggccgcgggaaacttaccagcatcctggaaaagacaaaaccagtggggtgcacttggcctctcctggctgcagggtgtggccgcccccacctttcaccttcccatccttaggaagcaaagtgacccctaagcctagacaaagctctcgaaagcccaaagcctcgggcccaccggccagctccccaccccgctgctgggccggacaggtgtaggggaggcggacccgccccgcagccgactcacccagctccagggcctggtcgcacctgagcagcgcggcctccggctgctgctggcgctgcaggctccgcgcctggcctgccagcctgcgcagccggcactcggccgggaagcacttctccagcaggccgctcagcaccacgttcacgcgccgcacctgcggccgcgcgggccctggctccacgcagcgcttgcagactgtgagcccgcagggcagcgtcaccggcttatgcagcagccgccggcagcgcgggcagccgagcaggtcgcggggcgcgcggggctccggggccggccctccctcgccgggcgcctcgggctcgccgcccgggttctccgcggacagcggccggtcgcgcaggcccacggcgcgcaccaggccgcccgccagctcttccagctcctccggccgcagcgccccgagccgcgcggcgccgcggaacgcgcccagggcttcggggaggcggccggcgcgggccagcgcgtcccccagcctcaggcacaggccgcggtccggctgcgccagcccggctagcatggagcgaaagagctcggctgccatctcgtagtcgcccgcgcggaaggcctcgtcgccctcctctaagcgctgggcgatcggctccgcgcggtcgcagccaggacactggggcggcggcggcggcgggaccggctcggggctcatcaccgcggggctgcgacgacgcgggtccggagcgaaggcgcggagcagggaggatgcgctgctgctgggaactggccggcgggagcgcggtctcagccctcgccagcagccacgcgcgtctgggggcggcgcgctgcgagcggctgagaccgcgggcgggggcgggcgcctggcttgggcagcgtcctcagcgcggtgtgggcggcgagccccgcagggctgcaatcgttccggggtgggggccgggacaggcaccgcgggcgcaatctgagcccctgcccacgcgcagcggcctctcagtcccgccggcttaggtaacccaggtcgctgcggtaacgcagtgaccgcgctccaggtccgcgtctcttgcgatgcttcccccactcgcctgagggctcctgcgcgactgcgcgcgcgtcctctgcctgccgcctccccgcagaggtgccggggccctgggagcaggtggccttggccgcgggctgctggcgcgccggcaccgcggcacctgctcttccccagaggcctggccgcccccacaacctgtggctccgcttaagcaagaacccaggaaaagtcaccaaacgcatcacgcatctctagcttcgacttaggaaattgtcctaaatgactggggaggctgaagtgggcacccagaggccccgcctcagcgagcttcttctcttaactctaggctgaggcctttgagaactcattttaacagagaacgtggagggccaagagaaccagataccaggaggaggagggattcgagaagtggacaattgtattgtgcccttgattaggaagcagaaatagaattagaaatagaattctaattaatagaattagaatagaaatagaaagggtgccctagtatggagtactgcagacataagttgctggggagagggagctaaac

SEQ ID NO: 6 - DNA, 68 bp, Homo sapiens
ggccgggacaggcaccgcgggcgcaatctgagcccctgcccacgcgcagcggcctctcagtcccgccg

SEQ ID NO: 7 - DNA, 26 bp, Homo sapiens
gggattttatttagcggttcgtattc

SEQ ID NO: 8 - DNA, 21 bp, Homo sapiens
gaaccgacctaaaataccgcg

SEQ ID NO: 9 - DNA, 27 bp, Homo sapiens
ttggcggtagaaatcgggagtggggtc

SEQ ID NO: 10 - DNA, 26 bp, Homo sapiens
tttgaggtttagataggtgatttcgc

SEQ ID NO: 11 - DNA, 23 bp, Homo sapiens
cgaccaatcacgaaaactacccg

SEQ ID NO: 12 - DNA, 19 bp, Homo sapiens
cgggtgggtaggcgcggtc

SEQ ID NO: 13 - DNA, 18 bp, Homo sapiens
cggcgggattgagaggtc

SEQ ID NO: 14 - DNA, 18 bp, Homo sapiens
accgaaacaaacaccgcg

SEQ ID NO: 15 - DNA, 26 bp, Homo sapiens
cgcgtgggtaggggtttagattgcgt

SEQ ID NO: 16 - DNA, 50 bp, Homo sapiens
gattaaccaacgaaaaaaaacgccccaaaaaataaaacgcaacgtatccg

SEQ ID NO: 17 - DNA, 50 bp, Homo sapiens
cgaaatacgacgattaaccaacgaaaaaaaacgccccaaaaaataaaacg

SEQ ID NO: 18 - DNA, 50 bp, Homo sapiens
cgaccgaataaccgaacaacgtattataaaaaccctaataaaacaacgcg

SEQ ID NO: 19 - DNA, 50 bp, Homo sapiens
ctacraaacaattaccttctacracaatttcaaaaataaataatacraac

SEQ ID NO: 20 - DNA, 50 bp, Homo sapiens
ctacrcaatcttctccrcttactttcaaaaattcraaaaatttaaatacc

SEQ ID NO: 21 - DNA, 50 bp, Homo sapiens
cttaaaacttaaacaaataaccccgcgaaacgaataaacaaacgcgaccg

SEQ ID NO: 22 - DNA, 50 bp, Homo sapiens
aaaaatacttcccgaccgaataccgactacgcaaactaacaaaccaaacg

SEQ ID NO: 23 - DNA, 50 bp, Homo sapiens
aaaaataccraaaccctaaaaacaaataaccttaaccrcraactactaac

SEQ ID NO: 24 - DNA, 50 bp, Homo sapiens
atataaacgacgaaccccgcaaaactacaatcgttccgaaataaaaaccg

SEQ ID NO: 25 - DNA, 112 bp, Homo sapiens
cggaagctgccgtcgcttgtgtccagaacccgtcttaaaagaacccgggccagcatccccagtcgcgcgccctcggcccgcgtgagctctccgatgcctgctctggctgtgg

SEQ ID NO: 26 - DNA, 127 bp, Homo sapiens
cgtccgcatcggcaacgtggcttcagacctgggcatcgtgaccggatcgggtgaggtgactttcagcctggagtccggttccgagtacctgaagatcgacaacctcactggcgagctgagcacgagc

SEQ ID NO: 27 - DNA, 113 bp, Homo sapiens
gcgccctccggcgagaggagctgaccccagggttgctcttgggggccgtgcctgccaagtatcgcgggacttgcgcacatcgcagaagcgccagctccagaagcagctggagc

SEQ ID NO: 28 - DNA, 146 bp, Homo sapiens
aagagtcagggtctaataagcccttcgtcggctccggggactccctcgggctcgggctccctgctctagctggagacctcgacagagcgccggccccctgactgcccgcgaagcgagacgcggggcgccgggtctagcgcagtgag

SEQ ID NO: 29 - DNA, 95 bp, Homo sapiens
cggacgcggagggaatgcagctggagctcgagaactgcggcggtcgctcctccgggcgcgcagcgcgggcggtgcggggaggtgcgggcgccgtg

SEQ ID NO: 30 - DNA, 91 bp, Homo sapiens
gctgccgggacggagggggcggcccccgcgctcgggcgctcggctacagctgcggggcccgaggtctccgcgcactcgctcccggcccatg

SEQ ID NO: 31 - DNA, 81 bp, Homo sapiens
cggagtggtggccacagcgaggcgctcgggagaggcgcctggaggccggcagtgggggcgcgccgcctgagcaggggtgcg

SEQ ID NO: 32 - DNA, 94 bp, Homo sapiens
gcgcgggctgactggttctggccgtgcctggcgggggccgggctggtaatgtagcctgcgccccaggtaacgcgccgattgcattaacccagcg

SEQ ID NO: 33 - DNA, 98 bp, Homo sapiens
gctgcgaagagcagagcgcgcagcggggctggcggcggcgcggaggagcgggaggaggagcgggaggaggaggagagggccgagcaagggaggagcgg

SEQ ID NO: 34 - DNA, 152 bp, Homo sapiens
acgcttagtaaacggccgcagaaccctggtcggtttgacgcaagaccctggcggatcccagagccaaagtaagcgctcacctccgccggacagggtaggtggggttgccaacccaggagagcgagggaagggggacggcagggaccactcgg

SEQ ID NO: 35 - DNA, 85 bp, Homo sapiens
cttgaggcttagacaggtgaccccgcgaagcgggtgggcaggcgcggccgaggggcgggaggcgggcagcctccgtgattggccg

SEQ ID NO: 36 - DNA, 127 bp, Homo sapiens
gcggcgcggccggctccgtgccctgcgcccgaggcccgcgatgcgcgctcgttagttccttggccggtccgggccaggacggctcaggggcgctggggctgcgagaggggctgctagaggggctgcg

SEQ ID NO: 37 - DNA, 143 bp, Homo sapiens
gaccctcggtgggcagcgcactccagtcttcccaggctagcggctgcagggagctccggcccgcggcccctccgcctcaagtctgggagctgccggtcccactctgtctttgcctatggggattcgagagtttcccagcggcg

SEQ ID NO: 38 - DNA, 153 bp, Homo sapiens
cgggcactgcgctcttggacacgtgggtggcgcgcggccctggacttggtggcgacggaccctgcccgaggtggggatcccaggctaaagaggaagaagagcctccggagagggacagggcacagtgactggcggcctccggagagggacagg

SEQ ID NO: 39 - DNA, 144 bp, Homo sapiens
cggtggactctcgagtctcgcttctgcaccctgcgtccctagacatgaatgtgaggagggtggaaagcatttcggctcagctggaggaggccagctctacaggcggtaggatgcaataagcttgcgagcgggcgcccgcttccg

SEQ ID NO: 40 - DNA, 151 bp, Homo sapiens
aggcggcgaagatgtggtaggttgcagagcatttggaaatggggtgctaactgggcgggtgcatgggagcaccgagtagcttgagctggcaagagaccttaaagggttacatactgcgaaaaggaaaggtaaatgttgggcgctggtgctg

SEQ ID NO: 41 - DNA, 112 bp, Homo sapiens
cgcgcggaggagacaccgctgaggggacgccgctgagggcgccacgcggggggcgcggctgaggcgcctcgaagggcaagcgccaagggggcgcgggcgccgccggaagctg

SEQ ID NO: 42 - DNA, 115 bp, Homo sapiens
aactcggcggtgggagcgggtgcttggtttgcatcagggatgaggggtggagtagcgaaagcgcccggagcgcgcactcactcacactccccaagccagggcgcggccgctctgg

SEQ ID NO: 43 - DNA, 118 bp, Homo sapiens
ggaacgatccaggcgcgctggagaccccgcgtgaagatgaaatgacggtagctccgggctgcttctgtaaaccggggagcgggctccatgcacccctttcccgtgtgtgtgggtttcg

SEQ ID NO: 44 - DNA, 107 bp, Homo sapiens
cgcggatcagtgggacggagttcggggttcggctccgagcgggcgggctggaagtgggggatccctcagccgcctccacgggccggccccgcgctcacgtcggttcc

SEQ ID NO: 45 - DNA, 67 bp, Homo sapiens
cggcgggactgagaggccgctgcgcgtgggcaggggctcagattgcgcccgcggtgcctgtcccggc

SEQ ID NO: 46 - DNA, 95 bp, Homo sapiens
gcgcagaaggcggaagccacgcgcgagggaccgcggtccgtccgggactagccccaggcccggcaccgccccgcgggccgagcgcccacacccgc

SEQ ID NO: 47 - DNA, 5869 bp, Homo sapiens
aaagatcccttagtgggtaacctcttcctttaggcagtgggtcttagactccccgggatgattccagaagcctctgataatcaaatgtactcaatccatggatattggatcagtaatactaaattaccagtttaaaaccctaaagtcagagaagtctggtgacctgctgaggatcgcagggctcctcttacttgcttgcactatagttcctgattctgtgaaaacaaaccaatgaaccccaactcctcaactcttaaaatgcatacagccagatacatatggacagcaagagtacagacatttaaaaacagagcagaaaaaagtaacttgaacagaatgtggcatttcccttggtctgttgctaacggcccacatacaacatgcttaaaaaatcccaccctggcacttattttcatcatcatttaaaaatgattcaaatggacatttagaatgcatggcattgagcatgtatcccatctggggataaatgaaaaagggcttcctcgagcagggctgtgtttttagtggttatcaagctaaggttaatgtgtaattggtctcttctctttggtcatgcttttaaagccaaacacaatattttaattgtctattttcagtactccaaagccagaagcattaaccagttctttttgtaagaaaatagctatggacatgatttgtaaaatgccttcaagatttgaagacatgaaagaaacatgtaaaaaggaattacctaattttctattattcacttttttaattaacccagaagccaatcacactttgttttactaaacactggagactcctgcttcctagatgtcacctttggggtggctgttttctgaatattgttccaattagagttcttcgcacaaattctgaaggttggatatcccacatacttatacatactctgcagtataattttaaactccaatatactcagacaagagtccataaccagccaatgggctatgaacctattttgatgctgcctgctgtcagcaaactggtctattgataactatcctaacaaagttaaaacaatgttcatggtaacactggaaagctgcaagaaagtaattatggctaggtgaaatgaaaaactgaccaatcatggaatgtattcgttcaccgttctaataagcccagcttttctcagtctcagggttttggtcctgaatgttacactgtagacattgtgtgttaatcgtttctccaatctaaagatgggaattatcctatggggaaaacatcaccttgctttggcctttacttccggaggattttaatgcttcattgctaacattattttagtactgcacacctggacacatcgcacagtataatgtaatatatgtagtcaggttttggagaatttatttcaatttacatacaatcctccaaatcctctggcgagcttgatgtgaaatctgacacagccacatctccctagcaggatcttcttttgctctctatgttaatagccaaaggctccaaggatctctccctggagcgcggtggggctaaggagtcaggaagaggggtgactgcggggcttgttgcgctgaagatttacaatgtacttcttgcaggcggctcagcaaccccctctgtggctgtgggtgtctggtgtgacagagcggagaaaagctctctaatctcccagtgcctgcctctgccgctcagtcgctcccctccctcccgcctttcctgcctcctcgcgcgctccctctcctttgcctttctctgggagtttcagtccctcaatgcagctggagtagccctggcaatctaaacccgaagcctgtgtatatacacacacgcacacacgcgcacacacatgcacacgcgctcggacatacgcacttcggctctcccccttgggatatggagcaaacccagagcttcatctacctgaaggggatggcccggccggacacttctttctcacttgtcatctccggagggcagagcaagactggaatgagggagtctcgctgtcgggtctgcaacctcctgccgtttgccgccaccgccgcttaaagtgcccagggtggagaaggcttttccaggacggatctccgtcgtagcttcgcggccctgggacttcctcctgctggttcactctagtctctcccacctcggccatccttttctctccagctgcagggtgtatggctcgggtgcctcccgtgctgtggccgccgtcactgctgctgcgagcacattccacccagaggaccgaacgccgccgctggagtggggggagaaagcgaaagccctaacttccctctctacaccctcgctcttcccaccgctcccgtccttctccctagccgagaaccggttggaaggctcccgcggaaagcgaggcgagagcgcggcgcaggggagggagagccgggagccgcaggcgcgcaggggcgggggcagcatgtgccccgccgccgggtgctcgtccgagaagtagcgcgcgctgggcaagcaagacgctttccaagttgggcgcgtcccagagctcacagcccggtgtccagagtgaggcggggctgatgggggtcgcggaagctgccgtcgcttgtgtccagaacccgtcttaaaagaacccgggccagcatccccagtcgcgcgccctcggcccgcgtgagctctccgatgcctgctctggctgtggcccgggtggcccgcccgcggggggtgccagagggggcgggggaggaggaggaggcggtgtgatggccctggacggcgagctgggggagcaagaggaggagaagaaaaagaagaagaaaaagaagaggaagaagaagaaggaggaggaggaggaggaggagggggccgagaagagcagctcacccttcgcagccgcgatgggggaagacgacgccgcgcttcgggctggcagcagggggctctccgacccgtgggcagactcagtgggagtgcgaccccgcaccacggagcgccacatcgccgtacacaagcggcttgtgctggccttcgctgtgtccctcgtggcattgctcgcggtcacaatgctcgctgtgctgctcagcctgcgcttcgacgagtgcggggcgagtgccacgccaggcgccgacggtggcccctcaggctttccggagcgcggcggcaacgggagcctccctggatcggcccggcgcaaccaccacgcaggcggggactcctggcagcccgaggcgggtggggtggccagtccggggaccacgtcggcccagccgccgtcggaggaggagcgggagccgtgggagccgtggacgcagctgcgcctgtcgggccacctgaagccgctgcactacaatctgatgctcaccgccttcatggagaacttcaccttctccggggaggtcaacgtggagatcgcgtgccggaacgccacccgctacgtagtgctgcacgcttcccgagtggcggtggagaaagtgcagctggccgaggaccgggcgttcggggctgtccctgtagccggttttttcctctacccgcaaacccaggtcttagtggtggtgctgaataggacactggacgcgcagaggaattacaatctgaagattatctacaacgcgctcatcgagaatgagctcctgggcttcttccgcagctcctatgtgctccacggggagagaaggtatggagggaggcggtgccccgcgctgccccaccccggcgcgcggctcgaacctctgggcggcctgcgaccccggggacccagctggcttccaatacccgggaagccaggggtggggggaaggaaacgaaagcggagtagggcagtcagaactccggggtctcccagatgcctcggggtctcgctgccgccaacttcgcaaactgactcaccggtgccaaaagatgaatgctgccccctcctctagtcgggacctctcctcttcctgtcagagttccttagccatcgaaacgcagggctctttttctgaagggtagctgataatctgagcgatgccagagtgacatgaaaagcttgccaagttactgtcgagggaaaatacgtggcgctgaaggccaagacaacagcgcatccaggttagcatgtcagggcaggtcagggacacaccgttctccacccccacccacaacatgcccatcttccctgactattagtctcctgccaggtgcagagcagaggatgggctttggctgcgtcaagtttccccaaacttcaagttgagattcggagtgaaaagagtggtttccagagataggctgagaggaccgggacgggggtggaggttgccaaggccagagccagcgataagtggcctcgcgggcgctgccagctctgtgctaaggcagggcgactccggttgtttgtttatgagttgctgcctctggagatgattccagtggccaagcggtgggaaagctagttgtcagtctcaaatgccttgttagtgtctttgccgcgttttacatttgtgtagttttgtatttttataaaaaatttccgagaggccgtggagcttttcaccacttgtttagagctcagacctcataatccaatagaactgagttcgaatctctgctgttcatctaactctggtggaggtttgaggaaggtaattaaccttttcctgacatgactacttctagtgcttattgcacgcccatgtactcaatccgacaagaaccctatgaggtagacgctattatcagcattccctattttacaggtggggaaatggaggcacaggtgtgtcacatagcttgatcaagatcaccaagagtgagcagacaaagcacggatttagacccagggagtctggctcttgagcctagacttttaattctaacttggggataccaataataatattctacttgtaggattggtgtagacgtctatgagtgaatgcattacaaagtacttacccctgtgcctggcatatagagtatccaccataaatgctagctctcatgagtgtcttgtggcagtaagctctcaggtatattgttgaatgaataagtgattcttgttctttgctctaattcccacattgctaagataacttagattaattaaatgactgagggcagatgaacaaccaaagtgagcttaaaaattaaaccaacaactttagaagaggaggcaggttacagatcagtaatgaggcagagcctctccttttctcaggtgattctcacaacagcagaaaatgggctcggagaagcccagggccctggaggtgagcaggcttgtcagtggagagttaggacttgaactttggttctctaatccctggaccagacagagcttatggtgtttggaaactctcagaagaagttattgtgtaagaggctggggaaattgcaatgtgttgtgaaagtaagcaaaagttttattatttatagagttctatgaagtaacctcctataaataaaatgcccaatgctttagtagctgtatgatagttaaaacctcctataagtgaatgtataaattattcactttcttactcctgacattagatatggatattttaaacccccttctaccacttcctccaacatgttctaaagaatacagattaaaaatattttttaaacccagaattatgtcaaatacgggggaaaggcatagaaaacaaatttgcatactatgtttaggaacaagcttcttaggtcaaaatgtcaatagtgggagttgcatatcattactgatacatcttgctgcagtgaaataaatgcttttgaaaaatcctgttttttgagccatgccaaaatatgtttggcagaactgagcttgttttggtcaatgctttcaaattaacaggactcttaaaaagaccagatgtttctgcttgaaccgtaactgatttcttgt

SEQ ID NO: 48 - DNA, 7639 bp, Homo sapiens
ggctggagacttcctcccgctcgcaaagaggaaaccactccactgtgaaggacgcgctgcaactccctagtcatccgggaagaaatgtgcagcgcgctgaggaaaagccgcccactccttcgcgccccgctcaagtgtgcgcctggagagtgcgcgccagagccgcgagcaaatgctttgaagatgcggagaggcttattcaagtttatggtctggagagggaagagggagtgcacaacgtttctgcccaagcggctgcctgaagtttgcaaaatcttagtaaggcgatgaggggagtgagtgcactgcaccatattgccgcgcatcggaatgcacggcgcaaggccgcggagtggagacggcttctcccatttgcctccaacctgggaaaaaggggaaccagggtcggaggtttggagagaggcaggcgttccgataaacccgggttcttgccaaatgtaagagggatttggctttactgccacttagccggccgcgaaggcgctgcggagcccacccccgcccccggctcgagactgcggcggtgcgcactgagcctgcgcgcggggcttttctcgctgatgctgcagatacagagctgagaggttcgggtgccagcagcgggagggttagcgggaaggccgggttccctgccctggcctcggaggaggagccaccacccctggggtgatgctactttcacagactggaaaatactcgccagctccctgacacacacttagacacacttccatccaacgggcatctacgatcccctgtagggagagcttggaaatctctgccgcctctccctcctcattcctgttcctgcgcccgcccaaacaaccgcgcgctacatctgctcgttgactttattggtgtttgtcttgtttaaatcattctgcctcgtcacccaggtctcgtaaagaatacccacactggacagattttattttttcggctttaaatttttctttgaggggaggcccttaaatgtgtatggatacgtcaggcgagctgaagactaagcaaatcccagcagcggccctttctctctttcgagatggtaaaaattctccccaaatacctacagtccgtctcagtcgtcttaatcctagccgggcctcttcgtttacagccactgaaataaatgagtaaatggctcagttcaaaaacacaaccagacctgagatctgatgaggccggctgcggtggtcgtgaatgaaaattggagaagccttgcttcaggaaatagggacagtcaagggacacaaagtcaaaaggagagatgggaaagctggggacactttggtacacaaagttgccattaaagtttatttcccttttatccagataggtgacgctacactttgccagttactaaaggctgcaggtttctgctcagaaaataatatcgaggtttccaacatgtgtttattccccaggcgcgcacacacggtctacccccgcaagcaagcacgacgacaggaatgtcaagcatttaaacaaagacagacatccttcaccctccacccaccgccgcccctgccccccgcaaccatccaaagtctgagctcccaaaaaaggatctttctccagctctctggagtttgatgttctctcttcactactgcagcccgtcatggtttttcagaacagagtggctttctgggcgctcgtaggttccccaggaccccgataaccatgtcctaaccaagatcctaaatcgcgaaccggagacactttccggggggaggaggacgtcgtctgagagggagggagaaggagaaacctatcctgacaccagctccgaaagctctcacgcgtgaactgggcattagtcaagggaggaggaaatttaccagcccgagggagggaaaacaagtcccccagagaagacggcggtactcgctagggagggggactgggctttggcttaaagaggactggctgggattgggcacactttgagcggcccctggagctgaggctgggctttcccgagcgcggcggctggctgacgctccctcccgcctccttatcccgggccctcctccctcgctctctcctagtgtgtagtttcggcggctcggctgctactgccgccgccgctgcagcagcagcgtcagggacaagcctgatccgtgagcgagcgagcacccagcagcaaccaccactttgggcaacttgcgggtttcctgctcgcgggtagcgaacgggagattcccactagcggttcagtgtctccgagccgctgcagcccgaggggcggcgggagcgcccgcaccagactctcccagacgcgaaaagggacgagacaactgcggaattcaccagccgtgccagggcacttccgaggccacaaccgactgacactttttctccccttggcaaactggatttttttttttaagctacttggcagctgtctctgactccacctccccctccacccccgcccgaaagcctttggcttttcttcggaaatccagacagaattgggcatctttgttaattgccgttggggacgcccggccgtgcgctttcgccggctaacgtcgcctgtgctccgagcctggtttgctcacctttgaactgcaaagggatcaagttcagcttgagttccctgcattgggaaggagagagagcgtgcaagagagcgagtgggagagggggaaaggggaaaggagaggagggaggagagagaggagggaggagagagaaagggagggagggaggggagggatcgagagagagcggggagagagaggctgcaatctcctccctgaatcgcgcacagcgctgcagatcccactgctccgacatgcgggccgaatgcaggtgagaaaaggcacggactctgcggctgcgaacccaaacttgggcaccgcacggtgcgcactgctcagccttcgcccccgtgggcgaaaggctgctgcggtttcaggcggctgcttcgtgactaatgaccttgcgcagagttgttaagaaaaaagagaaacccgcgctctccggggtgagaagggactgactctgggcgtctctgaagatggctcgggcttctctttggcgcgccggggggaccctgacactgaccgctctgtgacgcgagtagtctcccctgcaccgtgcccgaagcgacgtgccgggggatttttcattctcgatctgttgactggctcccccgctgcatgagcagagtcggagttgagactggcttgttgctggccccagcgcctggtgcaggaagcgactcacgtttgtctgggtggccggagccggagcagagcctgggtttggagtgagtgcctggaacgtgaattggactcaactcgagtagcagcaaagaccagcgggctggcaggcgggggaggctgcaggctcattccccacctcttcccagccccactgcccgtctgccggagcggttctggccccttccgacagagcggggactagagccggggattctccgcccgctgaggggatgactctgggttgggggagcgccgaacccgcggcgcgcagtgtcccgtgaactgtgagtactgcgactgaacggcggcaggcgagcgggcgattagcacccattgcatgaattatgaaacaataactttcggaagaagcaggaggaaaaaaagaagcatctatcgctgccctcccacccccattcccggccaactctccacgccgcttttgccccctccctcccctccctctcgctccttcctttccgggagaggggagaggactcgggggagggcaggcggccggccccggaggaggggggcgccgagggggctgtggttagaaggagcagtagcagcagcagcaggagaagatgctgaggatgcggaccgcgggatgggcgcgcggctggtgcttgggctgctgcctcctcctgccgctctcgctcagcctggcggccgccaagcagctcctccggtaccggctggccgaggagggccccgccgacgtccgcatcggcaacgtggcttcagacctgggcatcgtgaccggatcgggtgaggtgactttcagcctggagtccggttccgagtacctgaagatcgacaacctcactggcgagctgagcacgagcgagcggcgcatcgaccgcgagaagctgccccagtgtcagatgatcttcgacgagaacgagtgcttcctggacttcgaggtgtcggtgatcgggccctcgcagagctgggtggacctgtttgagggtcaggtcatcgtgcttgacatcaacgacaacacgcccaccttcccgtcgcccgtgctcacgctcacggtggaggagaatcggccggtgggcacactttacctgctgcccacagccaccgaccgcgacttcggccgcaacggcatcgagcgctacgagctgctccaggagcccggaggcggcggcagcggcggcgagagccggcgcgccggggcggccgacagcgccccctaccccgggggcggcgggaacggcgcgagcggcggcggctcgggaggctccaagcggcggctggacgcatcagagggcggcggcggcaccaaccccggcggccgcagcagcgtgttcgagctgcaggtggcggacaccccggacggcgagaagcagccgcagctgatcgtgaagggggcgctggaccgcgagcagcgcgactcctacgagctgaccctgcgagtgcgcgacggcggcgacccgcctcgctcctcgcaggccatcctacgggtcctcatcaccgacgtgaacgacaacagcccccgcttcgagaagagcgtgtacgaggccgacttggctgagaacagcgccccggggacccccatcctgcaactgcgcgcagccgacttggacgtgggggtcaacgggcagatcgaatacgtgttcggggcggccaccgagtcggtgaggcggctgctgcgccttgacgagacgtccggctggctcagcgtcctgcaccggatcgaccgcgaggaggtgaaccagctgcgcttcacggtcatggcccgcgaccgcgggcagccccccaagaccgacaaggccaccgtggtccttaacatcaaagacgagaacgacaacgtgccgtccattgaaatccgcaagattgggcgcatccccctcaaggacggggtggccaacgtggccgaggacgttctggtcgacacccccatcgctctggtgcaggtgtccgaccgagaccaaggcgagaacggggtggtcacctgcaccgtggtgggcgacgtgcccttccagctcaagccagccagcgacaccgagggcgaccagaacaagaaaaagtacttcttgcacacctcgacccctctggactatgaggccacccgggagttcaacgtggtcatcgtggcggtggactcaggcagccccagcctctcgagcaacaactccctgattgtcaaggtgggagacaccaacgacaacccgcccatgttcggccagtcggtggtggaggtttacttccctgagaacaacatcccgggcgagagggtggccacggtgctggcgacagacgcagacagcggtaagaacgccgagatcgcctactcgctggactcctctgtgatggggatctttgccatcgatcccgattctggggacatcctggtcaataccgtgctggaccgcgagcagactgacaggtatgagtttaaagttaacgccaaagacaaaggcatccccgtgctgcagggcagcactacggtgattgtgcaggtggctgataaaaatgacaatgaccctaagtttatgcaggacgtcttcaccttttatgtgaaagaaaacttgcagcccaacagccctgtggggatggtcaccgtgatggatgctgacaaggggcggaatgcagagatgagcctgtacatagaggagaacaataacattttttctattgaaaatgacacggggaccatttactccacaatgtcttttgaccgggaacatcagaccacatacactttcagagtcaaggctgtggatgggggagatcctcccagatctgccacagctacagtctcgctttttgtgatggatgaaaatgacaatgctcccacagttacccttcccaaaaacatttcctacactttactgccaccttcgagtaatgtcaggacagtagtagctacagtgttggcaacagacagtgatgatggcatcaatgcagacctgaactacagcattgtgggaggaaatcccttcaagctgtttgaaattgatcccactagtggtgtggtttccttagtgggaaaactcacccaaaagcattatggcttgcacaggttggtggtgcaagtgaatgacagtgggcagccttcccagtccaccacgactctggtgcacgtgtttgtcaatgaaagtgtttctaatgcaactgcgattgactcccagatagctagaagtttgcacatcccactcacccaggatatagctggtgacccaagctatgaaattagcaaacagagactcagtattgtcattggcgtggttgctggcattatgacggtgattctaatcatcttaattgtagtgatggcaaggtactgcaggtccaaaaataaaaatggctatgaagccggcaaaaaagatcacgaagacttttttacaccccaacagcatgacaaatctaaaaagcctaaaaaggacaagaaaaacaaaaaatctaagcagcctctctacagcagcattgtcactgtggaggcttctaagccaaatggacagaggtatgatagtgtcaatgagaagctgtcagacagcccaagcatggggcgatacaggtccgttaatggtgggcccggcagtcctgacctggcaaggcattacaaatctagttccccattgcctactgttcagcttcatccccagtcaccaactgcaggaaaaaaacaccaggccgtacaagatctaccaccagccaacacatttgtgggagcaggagacaacatttcaattggatcagatcactgctctgagtacagctgtcaaaccaataacaagtacagcaaacaggtaagatgtatcccaaatatatttaaatatcccagggagggctaataactctgagacagattatcttctgtaaagttttgtcttctcgtttttaaagttattaaaattttaacagtaggaatttaatgttaatttttgcaccattaatttagcaaaggccatctacaagaggtataccactgtattttgcaaattagaattaaggttcactaaactactgaaagaagtattcagagtaggcagtgtttggcagttctctttgcacttgacatccctaattcatactacatttttttgtttccagaataaatatgtgtgtattacttagatggttagttttcacctgaaattattttcttctgtggcaactaagtggataattacatccagagaaaaagtttctaaagttactactggtgtctatgcaaattggattccatagaagttttagctatgttatttacaatgcaggattttacagataaatagatgctgattcatgcagatgtagtactaagtctgagatattgaaatactgactactttgatgaaactgctggtttaaaaatatttactgatatgcaaatgtcatgcaaaactccactttcaatcttttatatctat

SEQ ID NO: 49 - DNA, 4544 bp, Homo sapiens
ttaaatctccattagtctggaatatttcctcagcttttctttgactttcatcacagtgacatttttgaaaagtataggcaagatattgtgtagaattttccttaatttaaggctttctttgctttctcgtgattgcattcaattatgcattttttaatctggaatcctaaatcaataatgttatgtacttctcagtgcatcatatcatgaggctcatgatatcacttggtcccattattggtgactttaaccttggtcagttggttaaggtgatgtttaccagatttatccactacatgtaatttattattcgtttgtaattaaggatctatagtatattataaaaacagcctcaagttatttttgtgtatgcattctccttgaaatatggcattatagtttctcccatcaagacatggactatattatctatgtttgggccatgtaacttccatggctaatgagacaccagcaaatgtacgtccacagaatctcagagtacttcacattggagcttgcactcccttgttgctttttggagccctgcaactacgactactgctagcctattgaaggatgatgggctagattaaacaaaagacaaaacattgcagctgagtcccacaccctcaagactaataagtatgccacaccagacaagtgagtgaagccatcctagatctctaaaccaaactgagctagccagggcagaaaccaacctgacaacccacagagctttgagaaattacagatgtttattggttggggctaataagttttggaatggtgtgttatgcaccaaaagccaatactttctagggtgatactttgggactacgtaaatatccaattacccaaataaattttaccaagtttatgattctgcctaagttaaatattatcacaaagactgcaaaatggaattgcgcctttttttaagagatgaaacatgtaagaatgggggcttatttgtctttgaaaaacataggactagtaagtagctgtatcagcatcagaaccccaatcttttacctggtccacagtttttctcagtatgccatgtagaggtgagtgacctgatgttaggactggaattcactagagagagcagtagagagacattccagagtgttatgagtgggaccctgtgtactcacacatgcacagaagcatctctggtaaatgctccataattgacggtagggcaggatggacaatgtaggaggagacattaatacagttattaacatagaaatttaaatgatacttgactcacgaaaagatataaagataatatctctggaaattgcctttcatgagtataatcaagaatggcattgacatactagattatgattaatagtatcatatgtttgtcaaacgtggctgtttaatatttttatgtagtcgttatatcctcccccaattctgccaaaaacatcctataagatgtgtgaagtgacactgtgagaagcaaggacccctgtcagcccttattacaaacaagacaactgacacccgaagggccaaaactagccaagagccacagtgaataaattgccaagcaggcacttgaactcggcaatcccaatttcctagaaaagttcctctcccatggcagtgggggacaggtcccaagtctttgagacgctaataatgtctgcgtcttaggtttctgccttgtgccaggcactgttctaaaatctttacatgtatttaaactcagtaccatgggggtattggtacaacacattaactgagacattcatacgtccaggtacttcggagtgggtgtgtgtttaatatcatacctacttgcctgagaaaggaattcaaagccccacttttttctttacctcttcataaaaatttgagagccacaaggaagcgctctgtgtttccagtactcttacagtgcgttcggtggcagtagtcaccgccaaccagaacactaggatcctgccagggtgtagatactgtaaccaacaagaggccaacctccgtcctttctgcggagggcgccgaagcgtcaagtaccccgcagcatctctcggctggagagccgacggcaagggtggggttgccggcgctttccccgagggcagtctctgatcctccagctcccctctaagaggctccctggtgcattgggcgaacgcggtgcacgcaggccggctttggcgttgtggggccccaaagcggccaggggtcgcgaggcgccctccggcgagaggagctgaccccagggttgctcttgggggccgtgcctgccaagtatcgcgggacttgcgcacatcgcagaagcgccagctccagaagcagctggagcgcgctcggtgcgcaccgccccagcccggggaggagcgggagcagggagggaggagggctgggggtgctggttacgtaaatgatactagcccagtgagaggcctccaggctggggctacgagaggaggagggaccacgcgaggaggagggaccgggaggcggcgggctggggtgggaggggggcgggggccggggagaagtgaccaggcggcggcggctctgctgaggtgacaacgtgctagcagccctcgctcgctctgggcgcctcctcggcctcagcgtccgctctggccgcgctcgaggagcccttcagcccgccgctgcgctatgaaggcccctctctggggctggccgaggctggagccggctccctctgctcgcgggcaagtgtgaagagagaggcgcgggcgggagccggggctgcgcgcggcgctcgcgggccggcgcgggttccaggtgagcgcggactcggcgagccccgcactcggcgcggccggccggcgcctgctgggcttgatctgaggctgaatcccgtgtgtggaccgccgttccctcttcgcgggatcgttggccaggatagcaggtctccgtctctttctcgcttcccctcttttcctcttgattgtctgggacgagctccctctgggctgccagagtgccggggctaggtgccgcaaagtcccgcggcgagtgccggggcgcggagccgagcgccccgggccggtcccagggcaaaggcaggggcgcgggcggacctggcgacgcggcctcccgggagctccgagtgcgctcgcttgcggggctgcaggtgttgcgggaggaccgcgtggcgcggaactttctgccgccgcccaccttctgctctgcttttctggctaccccggtgagttgttggtggcgggggggggggggggagcggctctggccccctctgcgttctggtgacgttcctccgcgggggtggctcggcctccaagcccagacaactttctcgcaccagctgcaggcagccccggctgcgttgcgggcgtttctactcgacccgacaccaggcgctccctgggtcttgggtgtcttcctcgacggttgccttcgtatagtgccccctttgcctcgctttttcagagtcctcttgcctctcattctcgccgtgtgtgtctctgcctctgtttctctttctgagccttggtcccctcagtccacactctccggccatgtatctgtttttttctcttattctgtttccctctttctttcttgggatatctgtgtccctgttaatctgtctttccttttgtctctccgttttgcaacatttgttctctttctgtgtctgcttctcgcagcttatctctgtctccctctgtcatctctctgccccgtcttcccgaacaccagccccgcgatcccgaagcgccttggttaggggagcaagccctggcgcatctaatgggtatctccaggtgcagagattttcctgcggggtcagtggggacgcggaggagacagcctgcccttgtccaggctgtcgtggacctgaaggaggggcgcaaggggttagagtggagggcaccgcagcaggctccccttgccgggtccgagggcgctgcgctgctgcgaacctgagcgcactgaggctgctactctggacagcgaaagctgcgcaagcccctcccgcatacccgattctttgactatggttagttgaatttccaggaggagagtctgcctccccaacctttatagccagctgcagaagaacggacagtgagcaggcaaacatagatgctttggggttcagggaaactaacctcgaaagggtgggagctttcttcaggcaaaggtgttgctagcagaggagaggagctttggggactgaggtatgcctgccttaaactaccatgcattcattgcttaggaaagcggtttccttggagagatcgtggtcagccttccctgaaacaacataacaacatagaggttttattcgcaaggctaaaggcacttggctgccaaagcccccttgaaatttctggtatccttgaatattttcagctttatgtgaaagctgcctttcctcagagtctcagtccgtctctctctctttctctctctctctttttctgtgtctctctctctcgctgtctccctgtctctctctctctgtctcgtttgcgttcatgccctctagtgttaaagaaacctgctggttcgag

SEQ ID NO: 50 - DNA, 6078 bp, Homo sapiens
tggcagtcacattcattacatttagtgagtcagactttccgccgtgcctaagtcactcccagctagtatggcaggtgtgtgatgagtgacgttagctgtttcaggagtctaaaaaaccgtgtaagtttaaagcagccatggtaatgccctactggcctgaaagagtaaacccaccctccattaatgcctcacatatgacttctgtgttggagagaaaagaatgcagaacatgttcttttaaaaaataaagcaaaggcttattataatcagaggatttgttcaaaatatttataagtcactttaaataacacagttgtttcctatttttgccattggaaatagtgggatgttaaggtagaaaaaataagaagaaattagtctttatgacactggagatttcatctcttttttaaaaactgcccagaaataacattaagcatctatctcaaaaccacaaaaccaataaatacagagttagaaatgccactacatccttattatcccttggttagtgtgccttgccctttttctctgttcacagttgcataattattttctccctggagaacatggaggtcaatctaaagtcaagactggctctaaatgaatgtgctgtctatgatacctggctctccttccaacaagacaaatgtggttgtgctacataaacacacaggagaaggccagtcacactgcaagtcacaaatttacaggcatatgaaataagacatcccagagtccgtgactgtgaggctgataactaatgagctctccaattagaacttggagaactggttatacactttggaaacagaataaaataatcaaaacttttaaggtcagagatacagaatttttcaaggtctaagattcatactttggaaatctactgcacactattgaggttcagggtctggaaaccctaaatttaaacgtcttccaaaaagcaaaaaaaaaaaaaaactcaagcccctttgtaagagttctgtcttttaaaagtttgttaacttaggtcaacactaaatccgcgagaatgctcctttcgtatctaggttatgtggttgttgatataaaaccagaaataagaaagtggttttatataaatgcctaagaaatcttgatcttcataaggcccaaacctcaagcagagttaaaagcctgataatttgcataataacatgtggtctcatctggattttttacagaaaaagtggttttgagttatctagataattgctctactcctcccctcttccaatttgtattaatagacatagaaagataaaactttcaattatctttgaaatctttatcattattgcccttcaacagagtctagaagtgaagtctgaactgactcaaaccggttgagtcttccccactattcaaatggggccgcgtcccgattatcagtgcaatgtgatcacttgattttaaacgcaactatgcgtccaaatggctgagtgcaagaggtcggccggagatagggccgagtgcgctgccccaagctgcgatccctgccaagcactcacggacaatgcactggtttcctccagggagcgtcttccatccagggcagcagtaggagtggaatctggagccgcacacgttgggcctgtgatggacaagcgcggtcacgtaacagataggtagagggatgcagcaacatataaacggtcttcgtaagcaatgccccgagcgagtagatttcaggacaacaaacaaatcaaaattacacttcaaattattggagatcgggaggcagacgtggaagcaaaagagctagatgctggaatgcaggagggtcagaaatgtctaaacactgcctttcatctgtcgtcccttgcctgcctttttaaagggcgaaaagaggcatggaaacatataatttgctactattctagcttccagatgcagagaacaaagggaccattccgttccaaagaatccttgccctccttcccaaatccattcctccttggtcaactgttactttggccccgacgtgtcaaggaaagcgcgtcgcaaggtggagcgctcgggttgacgatgagaaggtgagctgtgaaccctgcacacgtcctcaaacaaaaggctcacgagcggcggccccgacgtggggagcaagcaggcttctctggcccctgcagcaggcggacccaggcccgcctgccgccggacattcagaacctgttcccgcagcgcgagctctgcacacgctctgcgatcggcacccctggaccccctaaccacccgccgcccaacccccggagagagggctttccgcccgctgagctgcggggttgggaaggaaaagggggacgcgctcctgggggtctttggatattctggcaaagggggctgcagctctaggctccagctaaagggtctgggacggagtgggccagaaagccgccaaactgaggattcccccctcccccaagccggagccctaggtgcggagccgcttgcccacttaccctcggagcacgtcctgctgtcctcgccggcggacgcggctggccactgcggcaccctcctcgcgatactcgggcgctagaaacccgccttcagagcctgctgtagcggaccgaacctgttgcggcggcggctggggccggggcggcttgggcggaggaggctgaggctggccggccgtgccctgcgcccagagcaccacacagcccagccacaggaagtagagctggagacacagcctccgtcttctccccatcgccggcgccgaaagcgcgcggccgtagacccgcggagagggagtgatcaaagacaaaatctgcgcgcctcagaaaagagtcagggtctaataagcccttcgtcggctccggggactccctcgggctcgggctccctgctctagctggagacctcgacagagcgccggccccctgactgcccgcgaagcgagacgcggggcgccgggtctagcgcagtgagcggcgaggcgcggcggaggtgcagccggcagccccgagcggtacacgttgcataaccggcctaaagccccgagcgactccaggaccgtcagcgggcgggggaggaaattacaaaagccctggcgtaaaatttcaaaaaatgtgaacctcaaaagaaaaaagggcctgcacgcagagctcggcggattcccgtgcgctccgaagacggatattggaaagctgcaaaagtcaccagaaagaaacaggcaagcaaacaaaagtcgcccccagaagaagaggagggttccctccgggctcgctcggagtcccacagggcaacgaagcgcgggtagcggctgcggagccgggcggaggtgcgcggggccggggcgtgcggccagcaagagagagggcgggaggctgggcgctggagaagatgcggcggtggagggcgcggcgcgggggttttagaggcccggcggggcgcggggcgggccggcgggcggcggctgtcagtgggggcgcggccgtccacacgtggggctcaacccggactgagaggccggcgcagagtgggcggggacagagtaagggagagatcgggactgaggaacgaggggaaccagggttgggtgaggggaagaggaaggaagggggggagggcgaagggagggaagaggacggagattggggagaaggggggcgaacccgagaggaaggccgggctggaagagggacttggaaagagggattggcgagcccgggtgcgagaggaaatgagcgggggatgggggcgaagttagggcaaagagagaagtgggaaaggagagggagggccaaggtccgggcgccgagaggaggggcccggggggccggctggagggggcgaggcgctaggcggctttgcttcccgtccccgtcaattggccgctagctctgttttgactgcctgtacaaagcgagtagacggcgggggatggggcgattagggaccggatttgggaaaggaggcagcagccccttcccctccccctccgtcctcgcccggccgcggaccagggactagggtgcctaagcgcgccaggcttggccggacccgcagctgaactgacccgcagctgaacttccccgccggacagaggccgcaaggttgaggatgagaacgggtctttctgtagggtcccggcccctgacctgggatggggggattcgcagtcagccaggaatagaggctcacggggctgttggttgcagcctcaatccaagaaggaggagacctcccctttgccctcctacccccaccgccccaccgggagcggcgtcagtctgcgggaccaaattaggggctgggagtttccagattgaaatgcgccctccaccaccaagagggagctcacgcggccgcggggggccgaggcagccagccggcgccgcggtcctggggagtccgtgcaggggtgtgctctggtgtgaaacctggcaggactctgcctgggcttgcatgcagggaggggttagagactctgaaatgtactagcgggcagggtggtgacaaggcgggtaaatgaggctcggggcatgcccttgcttcctggcttggaccccttctgattgattaaatcctagcaattggtgcaaccctctgtacctcttagagtgcctctcaccggcctgctctgggttcttcctttgtggccctcctttcatgaaatctctgtgaagtcctggtcatttctcatttagtggaatatcacaatttaaaaatcaaacactcctgaaaacaaggagagaccatccaagcacagtttgcgaaacagtacaaaagagcagtgtctggtttattcaagcctagaaaaccttagtttaaaggggaaaatgttgaatatgtataggagggtgtgtgtgtgtgtgtgtgtgtatatatatacacgtacatatatacatatatacacataacatatatatgatatatatcatatatcatatatatcatatatcatatatatgatatcatatatatgatagatcatatatgatatatcatatatatgatagatcatatatatcatatatatgatatatgatatatgatatatatgatagatcatatatgatctatcatatatatcatatatcatatatatcatatatatcatatatatcatatatatcatatatatcatatatcatatatatcatatatatcgtatatatcatatatcgtatatatcatatatatcatatatcatatatatcgtatatatcatatatcatatatatcatatatcatatatatatcatatatcatatatatgtatttctggcccaaattgcccagatgtgctatttgacagccaaaattctccagtaattggcacaactccctcctttatttctccatgtagtcaaagggtctgaaggtagtaccccttgttacagtattctcataaagcagaagaaaagataagagttacataatttcatgtgagacaccgatttgttaaaatccagagaaagagcaatacagttattttcacgtacttcgcttcaaataataaaattatttaaactaaaagagctaactaatgtaactatctcctggtttaaagcagtgttttagtcataccatttaaaacatcctgtatctgctttttttgttgttgttgctgttgtttaatacttacgtggcttcatggttcattcctgtccatcacattaaatctaaatgctctagaattgcctatgcctttaccctttgatccagcaattccacttttaggaacctatataaaagatacatagacaaaaaaaaatgaaatggtgcatacatttgactgttctttgcagcactgtttataatagtgaaagactagaaacacaaacttcccatgaacaagcaactgttgtgtatgacttcatctattcagtgaagtattatgcagctgtcaaatatgaatgaagagaaactctgtatgctcttagagagtgatctctagcatatacagtctactaaaataaaaaaaaagcaaggtacagatagactaggcagttcattgagacaaagtagaataaaggttaccaggagctccaggaaaatgggaaggatgagttattgtttagtgagtacaaagtggtctgcctagga

SEQ ID NO: 51 - DNA, 5036 bp, Homo sapiens
agagaggtgcatggatgagagaccacctcctcccttatccccctaactcctggcacccctctcctccccatcccctccctgccaccccagctgagaagcgtgggcctgagcgaggctggcaggtagcagggacattcggctgcatgctaatggccaaacagcatctgcagtgagacagacgcatgttatcacgcagatcggttgccaggggaatggatcaggagggcagccagctgggactgggaggggggatggggggcagggaggggctgtgtctgctgcagacattgctgcagacactggcccagctcatgggagcccgccgactcccccaccaccacctgccagctccaaggacactttttctcagtctgttttttcaaaatcaggtgtgtcacagcccctgggtgatgctgcccctgttcctcaggggtctcagggatggccaggacatccagggtggaaatggaccaggaagcctttgccaaatgtggctgaaggaaccaatcacagggcctggtgctctacccaccaaattcatctttccaggccggcgtggtggctcacacctgtaatcccagcactttgggaggctgagacaggtggatcacctgaggccagaggttcgagaccagcctggccaacatggcgaagccctatctctactaaaaatacaaaaattagccaggtgtggtggcaggcgcctgtaatcccagctacttgggaggctgaggcaggagaatttcttgaacccagaaggtggaggttgcagtgagccaagattgtgccactgcactccagcctgggtgacagagcaagactgcatctcaaacaaacaaattcatctttccatgtttgggagtccatgtgacatggagttggggtatggagcagccaaggcagaggtttaggggactccaagggccccccaagtcacccagagctcaaggggcaaaagcaaaaagagatggggcactcaccccaaagcttggcccatggaagaaacacctaccttttccagcaacacagcatgtgcagccctgaacattctagaaaatggctacaccagtcctttcacacagaatatgcagactacccaaaacacttccaaagagtacctctaaagtctggataacacaacccagaattaattaatagtccagtacatggacttctgacttacaaatccctgattcatggactccagaaaggggaagggtagaaaagtctccccgacgggtgagggagaagatgtttcccaactccactcttagccactgtgattttgaccacatcaagggtgcaattggctagaatgggaaccacacatgatttccaaataattaattcacttttgttattattttctttttcttttgcaatttaggacacattgaaaatctctgacacaaatcaacctgatggacaagagacccctttcctgtatttccaggaggaaataggtaatcttgtccaagtgcaattcccataaaattcgtctaattcatccctgagttgcaggttttctttgacctcatggtttttctttcacaaattttcattagatcccttgctctctctctccaccgctcttttatgaggggggagggggtgaaaagtggtggatagtattcaaccttcttagctgcccacgtcccatctgtccctcgaatagtcttaaatctgggtccttttatggttcccaaaatgtggcagagaggtgggtgctggctttctggcggtggggaagtgggggcagtagacacacaaccctgcttcccccactcccccacccacacacacaccccaggaacctgtcagctccgggtccattttggaaatcaccaccagagaggaaatggcagcctggcctcctgggctctaaattcgcatctccccaaagccagcaccaacacggtgggaaacccctctagatgaggcgacattttaaggcaaaaacgttgcccatcccgtgctccctgggggactggggaattgtggcgcgatgacggaccctggcgcatccccgcctccctgcctcgtcctcccctcccttagcgggtgcatggtccccaggcatcgacggatgcgccggccacccccgcgcggtttccagggcagggccagcctgggggggtggaagcaggaatccagggggtgcggggagccccctccagtgcccaggcgactttggggaacaaggatgaaaggagaggggaaagggcgctgcagtggcctcagccgaggagggagatgcgcgcagggcgcacggtgccacctccgcaatccctgctcccgggcccactcgcccgctgagacggttccaggccccgggcgggctgcggctgcagggaggccggattgcccagcgccttacctgcggccacccctgccggggcccagggcaggaggcaggcaagccagaggatgcggccgaggcgcgaccacactgcctgggccattgcggggcccggagcgggagaagcgtggggcggacgcggagggaatgcagctggagctcgagaactgcggcggtcgctcctccgggcgcgcagcgcgggcggtgcggggaggtgcgggcgccgtgctcgctcgtcctcgccgggggacgctctgtcgctgcgcgccgccgccgccgcccacagcccgctgcgcgaccccggcccacgtcagccgcgccgcgcgccctcctcctcctcccgccgccccttgctcccgcgtccctctccacggccgcgtcctccctctccgggtctcttcccacggccgcgccccctgccctcctggagtccacgatcgcccctcccagcttgcttcccctctaggggttccagagctcccttcctgcgccctggctcctccccgcatccctccccaccccaaatccacccccggccgcatccttcccctccactacccgaaaccacgccccccgcacccgcatttttcccttcccccgagcccctccccgccctggatccgatgccgccggctctgactcagcgtccccaggggcgtgcgctgcgctgcaagccgcctgcaggtcccttggggctctcctccaggtgccttctctctgcacgctcgtggctccgagatgctactcctcattcaagaactgttgagagcttaaggtgcgccaggcgttgggcgaaaggcctcgaaattcctcttgctgctgctgctgctgctgcttcctcttttcttttttttttttttttaatcttgagacagagtctcgctctgtcccagaatggcgcgatctcggctcactgcaacctccgcctcccgggttcaagcgattctcctgcctcagcctccctagtaagatgggattacaggtgcccgccaccaggcctggctaatttttgtatttttagtagagacggggtttcaccatgttggccaggttggtctcaaactcctggcctcaagtgatcctctcgcctcagcctcccaaaatgttgggattacaggtgtaagccactgtgcccagcctcttgccccttcttgatagaggggcctgagcacccggcccctgccgtgggcttgatgtcatcccccggtttaccctctcacagctgctgttaagatggggctcctgctcgtatctgggcccagggcaacttctttgtcactgagggccttgtgggagtcaggctactgcggtgaggggtgaccagcctacacagttactgccatttgccttagaggagcagggctgggctctcctagaagctggaggggcctggctggccttaaacgctggaagcccggtgggttctggtcccagctatgcttccgctttgctacggctacctgggcgagggatcctcccacacagtgagggcctttgacctcctctctggcccgtggagcacggatggaccatcactgcaccacccagtgcattgacatcaaggcgtctaaagcaggggtctccaagcccctggccgccagtaccagtccatagcctgttaggaactggctgcacagcaggaggtgagcagggcctgtgagccacgctgagctccacctcctggcagatcagcagggcattagattctcataggagcgcaaaccctactgtgaactgctcacgtgagggatcaaggttgcgtgctccttatgagaatctaatgcctgatgttctgtcactgtttcccatcatccccagatgggaccatctagttgcaagaaagcaagctcagggctcccactgattctacatgatggtgagtatgtaatcataatagaaataaagtatacaataaaagtaatgcgcttgaatcatcccgaaaccatccctccaacccctcattcgtggaaacattgtctttggtgccaaaaaggttggggactactggtctaaagggcccctggctctgggacagctgtgagcttccctaagaatgggccccactggccccactgctatgttcagggaatggagggagaatagatttgagtgagcccctatgaaaagttaaaagggctagacagggcagggcatggtgactcacacctgtaatcccagcgctttgggaggccgaggcgggtggatcacgaggtcaggagttcgagaccagcctaaccaagatggtgaaaccccgtctctactaaaaatacacaaattagccagactcggtggcaggcgcctgtaatcccagctacttgggagcctgaggaaggagaattgcttgaacctggaggcggagtttgcagtgagccaagattgcaccactgtactccagcctgggcaacagagcaagactccgtctcaaaaaaaaaaaaaagttgggggggtcggttgcggtggctcacgcctgtaatcccagcactttgggaggccaaagcgggcagatcatgaggtcaggagatcgagaccatcctggctaacacagtgaaaccccgtctctacttaaaaaaaatacaaaaaattagccgggcgtgtagtcccagctactcgggaggctgaggcaggagtatggtgtgaacccaggaggcagagcttgagtgagctgagattgcaccac

SEQ ID NO: 52 - DNA, 5986 bp, Homo sapiens
atcttttgtttacaaaggcatattgagaactttggtcatttttctaagagcatcaggtaaccgcagggtttggggtgttcagcaaagcccgtgattacacacacactggaccttcagagaagtgtgccatctagctacgaccacagccatttctcctggtctaccccgtccatcgggacgcactgctgcaggggaagactcacccatcatgcaacccctcactgcaccctggacagtcacttctaaaatgcaggccctcaacaaggtattgacgtgtgcattaatcaccttgattattacagagcccctctgcggtggaagggcaggtgctgtcattcctacaaatgcatagagctcaaagttcatcgctcacccaggtggaaccgggagaaaaagatgcgtttgtcgaaggcacccagcgccacgagctttcttcacaagccaagcagcccgcctaaggcccaggtctccctctcctcgtctcccactagatacacatggatgaagccagatagataacaggtagaggatagatgacaggcagatgacagataggcagatgatagatgatcgatagatgacaggcagatgatggatgacaggcagatcacagatatgatagatcacagatagatgacaaataggcagatgatagatgacaggcagatgacagataggcagatgatagatgactgatagatgacaggcagatcacagatatgatagatcacagatagatgacaaataggcagatgatagatgacaggcagatgacagataggcagatgatagatgactgatagatgacaggcagatcacagatatgatagatcacagatagatgacaaataggcagatgatagatgactgatagatgacaggcagatcacagatatgatagatcacagatagatgataaataggcagatgatagatgacaggcagatgacagataggcagatgatagactgatagatgacaggcagatgacagataggcagatgatagattgatagatgacaggcagatgacagataggcagatgataggtgattgatagatgccagccagatgacagataggcagatgataggtgattgatagatgacagccaggtagatcacagatatgccggatcacagatgatggataggcagatgatagatgacagataatagatgatagattacagatagacgatcgataggaagataatagatgataggtacatgacagatcatagataaatgacagagaggtagatgatagatgataaataggtagatggtaggtaatagataggtaatagatcgcagagagatgataggtagatgatagctgattgatacatgatacagagatgataggtacatgatagatggatagatcataaatgacagcagatgatagatgataaataggtagatgacaggttatagataggtaaattacagatgaaagataggcaaatgatagatgataggtacataatagatggatagatcatagataatagatgacaggtaggtgatagaggaaaggtggatgatagatgataggcgcatgatgatagatacctgacagatggatagatgaaaggtagatgatggacaggtagatgatagatgacagataatagatgacagattacagatagatgatcgataggaagatgatagatgataggtacatgacagatcgatagatgatacgtagatgatggataggtagatgatagataatgtggatgataaacatcagatgatagaaaaatcgatatctgtgaatacggggggtgggggatcagagagaagcaaatgataagcccaatgaggtaaatgttaatgaccgaatccacaaaaaggataaaaaggagttatttgtaatattcttattcttctaatttttgtgtaagtttgaaattatttccaaataaaaaattaaaacaacacgaacttaacttgtatctctcattccagtgctttccacttgctggggaacgccgagctctcctgggttggtcacgcgggcgccttgggaggccgcgcagtcccgcgtggggcgcggcggggcggccccagtggcaccgcgtgagtccccgcccagcgttccccacccgccgccgcgtttgcggggagagaatgacccccgttttgcaaacgcaggacacaaaacccgccacccagctgaggctgggacccacctatacccccgtctgcgtttttctccagctttctgttcgacctgatgtcctctttgaggctgcggggatccccaccaaaaagcactctgattttctccattttcagatgccccaacctagccccactggctacaaagacggcgcgcccaccccggacgggtctctggccgctttcttgggcttccgcagttgtttagaaaggcaggtacaggcggccgcaaaggcatccgccccttccgagcgcacacctggcccggtcctcctctcccggccccgcgccggccgctctcccccagttctctctactcggagccgggcgcgcactgagcaccccgcttcccagcccctccgcccacagatcggggaggcgcggccgggaggggggcgtgggccagggaagaggggaggagaacggagagagaggctgggggggtcgaggagaccagagggagcgcgcgcggcggggacagagagcccaggggcgaggagagggcgcggggcgcagcggaaggggaagtggggggcggtgaggaggggcgcggagcgggggttctcgggggaggagggagacgaggattggggcagcgcggggcggggacaggggcggggggcggagcggcggggcgcgtgggggacgggcgtgcattgagcgcgctccagctgccgggacggagggggcggcccccgcgctcgggcgctcggctacagctgcggggcccgaggtctccgcgcactcgctcccggcccatgctggaggcggcggaaccgcggggacctaggacggaggcggcgggcgctgggcggcccccggcacgctgagctcgggatgcggacgctgctgcctcccgcgctgctgacctgctggctgctcgcccccgtgagtgcgcccgcgacccccgccccacggcgcctcggacccggttctccccttccgagaagagcgctcctcccgccggtcttcgccttcctcccgggtgctggagcgcgggcggggtccgggagagggagcggggtcgcccggggtccggagcttcctcccggagagcgtgaagcgctgagctccggtcccgccgggttgcgggactcgggttgggaggctgcctgcgcccttccccgcgccccaccgtccggggtttgctggaaacgggatcgtttcttcctggacgcgtcaacgatgagctcgttcggggccgtcccgggagctgggagctgcgggcgcctgcgcgggctgcgcgtttcacggggagatcggggttggcgttggccgcagatgcctctcggtccctccctgtacttactggttagttttagtgtcggggggacgtggcgtcggttaaataaagtttccttttggaaatagttgctttttcccaagggcactggaggaactaactgctcataagtttgttgattaagtgccttcgcacatggagttctgaattattccctctgtgaaccttaaaaaaaaaggaaagaaaaaagaaagaaaaaaacccgagccttgtttcttgctttcctcttggagactgaagcgggtttgcggtgcgcgcggttttcgtgcgcgtcgcagccgaggcaggtctggatcgatccgcggcccctccgcagctgggaggctgcgctgctgacccgacagtgtcattgattaaccggcgacgcccggtctgcagcggggctcgagccacactccctggccttgcgcggaggcgcagctcggctttcctccctcgagggtgggcgctttgcggagcgggtccttggaaagtaacgggttttatcacggttaatgctgctttcattgctaaaatgtcctaaaagaagtcattgctgtgcatattccaggaacgtcaggtattattaatgtcttttacactttaacaaagtttttctttttacatcaatataatttccttaattctgaaatttaagccacttacttgtcctgcatgaaaacagaggacttggagcccaaagtcagtgcctggtgctggttcagtcacgagcaagaagcaagctcggggctcttttaaatggggctgaaggtggcagaagttccagcattgttttccccttaattctgttaggatattgtgttcctggttggaactctcattgaccatttcaatagtaagataactaaacatagatggccatagtacgcccaacagtatgattctctttagttactactttagtacaagtagaattttggtcggtggtgggggagggggaagatcaagaccaaaaggatgggtggggggagccctccttaagcccccaagggccgcttggcaggcctgtgctgacttcgcggagggcgtgaagaacaaggtagaaacggtgccttctgagaggaagcattgtgtagttctgccttcttctcacattagtagactgttaattactatctttgtggcacctttcgctcccctgatggatcctgactggaaatcgccccctcttctctgtggggccacaatggtgtgattcccccacctctgcctccctcctcatcttgctgatttctttgaatatctcttccaccccaaattctagtcaaaacgattgcagttttatctgtagttagagggttaagctttgaacatgtaccaacacctttaaaaataaagcccttctatcacggctctttcccgggtggaggccgtcctcggaggaagcggcacagtccacaggcatccgcttaaaccaaaccaaagggtgtgaaacggaggagccgggtatggagagatgaatggagtaaagagctctgttgcctccaaattagtaccgatgtattctgttagtaagttcattttcctgctcagaaaaaaaattctattagattttgtgtttgttttataatacctttacaaagtaaatagtgaagtttttaaaaaatgccagaaaaatcaaggtgattggttttaaatgcattttggtttggaagcagtttaaagctgtgaggtttggggttgtttttgtagaatttttgcttgcttttcttcttcttgtggaatttttagttattaatatttcttttaaccgtgaacaggtgaacagcattcacccagaatgccgatttcatctggaaatacaggaggaagaaacaaaatgtgcagagcttctgaggtctcaaacagaaaaacacaaaggtaaggcagaggcttggtgagtaagaactcccgtgacattctcacccgggaatgcgcctcactcagctgtggttcagggtcaccacctgcatcttaaaaaaaaaaaaagaaaaagaaaggccacttcccattgtttgttaatgaactactggtaaaactcagagagagttggataggtagggtcttcacacacacctttaaaaagataaccttaatccttagcagtaacagtttccatggtttgagatgatagcaacagttactaaaacaatttcatgggctttgttatgctttgcaaaagcagcttcagttttccaggggataagcatttatctgaattcttgcattttaaattcttagctataggacagatgattttgatctggttaaggatggtgggtacttggcagaaagcaaacaggaaaatgggactttggttgtgctgaagacgggcctgcggtgaacactgtgaacgtgcagatcgccttctccggtttgacagaggcatgctgggccatcatgctccaacagggcctgctatcaacctgtttatagactctgccctgggatagcacccggcctataaatagtagcaaggcctttgtccctcagctttctctgctctttgtca

SEQ ID NO: 53 - DNA, 5253 bp, Homo sapiens
ttccccattttataaggacacagtcatactggattcgggctcattctaaagacctcatttaatttaattccataaagaccctatctccaaataatgtcacattctgtggtactgggggttatgacttaaacatataaattttagggagacaaatttgaacctctaacagtactgaacatccaggatggaagaacatggtattaggttgagccaaacacagttgcttacgttttggttttcctcaccaggacaagaaacccccagtgcaggaaaattggagacatggaaaacagggcttaagtaaacacgcagatctttgccagaaccatgtttgtcacacagtgtggaagcaacgaatatctggtgaacaaatgaatgtggacttgttcttctaaatgacttgctccctcacttcttccaagtcttggttcagatattaccttctcagtgggaactagcctaactactgttcttaaaaccaccatctccccagtactcaccttcttttctgttactttctttctctctataacactgaccactttctatcaaactctgtaattcacttatttgtcatgttaattgcccatctctccctactagaagataagttccatgaactcaggaacttttcactgttgtgtttccttctctatgtaccagcacttgttacagcgcctgacacagagtaggccctcagttttgctgagtgagtgaatgaatgaatgaatgctgttgagaaagagctctggaatctcctggcatgctatttcaggttttgcttggatctcccctttggccgtcacagaacttcaatgctagtctttaaaggaaatctcaaagatcaagcgcctatctccaaagcttgaattctctcttaaactggtgacatcttgataacaaattgcctttgaatgtagaacccccattttaataataagagagtaagagattgaaactgtttaacaagtaaaatgtgctatggtatgtgtggtacggggaacagaagcagaaggcctgccatacgttagttcaaggggtagagtcagagaagggtgaacatactttggagtgcagcaggggcaggatggcacataattgtatgtcaatgcagagtaaaaggaccagaagatgataagccaagccaatgacagtgagcagcatagtggtacagagcaacgttttctaactttatgcatgcaccagccacttgggcatcttgttaaaatgaagattctacttctggggtggggcctgaaagtctgcagttctaaagtgttctcaagggaccgctctacgacgccatgctttgcatactaaagagcatggatttactagttacctgtgtaatcttcagtaaacgatttaacttctctgtgcttcagttatctcatctataaaatagagatcagatgaactaatcccagtaaaatgaataccattaagtgacactgattatcagttacttcacttgcggaagagtggagggcatgactaggaatggggtgggaggagggagtcaaagaagtcttagctgaattttttttttttttttttttttttttgagacgaagtctcgctctgtcgaccaggctggagtgcagtggtgcgatctcggctcactgcaagctccgcctccccggttcacgccattctcctgcctcagcctcccgagtagctgggactacaagcacccgccaccacgcctggctaatttttgtatttttagtagagacggggtttcaccgtgttagtcaggatggtctcgatctcccgacctcatgatccacccgcctcagcctcccaaagtgctgggattacaggcgtgagcaaccgagcccggccccagctgtattttatacattaaaaaagaaaagtatttaggtattcatacgtgggccgagttttctcctctctcatacaagcacattacacgcgaagccagattagttcatgaatgtgctactgcacggggtggctaagaaatcctgcttgcaaaccgctttgggtcctgcgtggagaatggtttcgagtgagagccgaaccctaaatccgtcttccttatgtggagctcaacgcgactctcaggtattcaggaagaataccttttgctcagcacctgcggagtggtggccacagcgaggcgctcgggagaggcgcctggaggccggcagtgggggcgcgccgcctgagcaggggtgcggggcggggagaaggccggcccacgtggaccgcggggccaggcagggacaggagcagccgggcggcccggccaatcagcgcgttccgcggcgggcccggcccctcctggtcagcgcgctagctgggctcggctccgcactgctagctgcgcgccgccctggacggggcgcaccgactgcgcgcgcggctgcgggcaaacatcgggagtcctgcctcagctgccgcttctccagcagcagcttcaggcttctcccgcaggagcttcgggcttctcctggtagagacgtgggaacttttcttctcctggcgaggctgcagaggtgatgggccgctcccggggctcccgcggggaggcggcacggtgagcgtcctcgggctccggtgcggcgatcagtacctagttccggacgcgccggtccgacttggatgccggctctagtcgagtaagaagggttggaagggataaggaggggcgagaggatggggtgggggtggatttggaccctgtatttaggtgctgtctcgtgggcagccgctgcctctcggctggtaccgagttaactcagctcggtgcagctcccctcatcccggctctctggggcgccggggagagtgcctgtgctgaggtcggcgtgcaacccgaagttggaaggggcactccgaagtaaggatgtgtggctggagagaggcagcggccgcttccagtttcggggttcatgtctgacagaatccccgggggtgctctgctgatgcggaggaggccactcgatgaattggtatagggggtggtgaactcaccgtgagctcttttctggacaagtcgaccttagcgcttcatccctttaatctgagctaggatctttctaggagcaagcaggtgggagccggtcagcgtccccccgcccccacccccaccctgaagctctggttgctaggatcttgcttgaagggcgcagcgagcgctcgggaggcgtcttccagctgggagcgcaaagcttcccgcccaagtggagaactggagcgggtctggaagttgcgtctctttccgcgggaggcatctcagatttgcccaccaaggtggcatctctacatttctttcgctctttttcccactttgccctgttaatccgctcctagaagagagggcatccttgaccctacgcaaggagctcgggagaggattggaactggaaagcttgatctcctgcacctggtgaggctgcccaagccagacacagtgccgctggtgcgttttcttctcgaatcagcatcatttaacgtttaggggctccacggagccttagtaaagctgtgaatcagctcctaagcagtatgtacatgcacgggttcacggactcgccgagacctagcctggtgcccatcccccgtcaccgagattcagggacccccctttggtttaacgtctttatccctgaactcttttcccttttaacttttcaatggtcttccctctgagacaagcaaggttgatgtgatgtgacaggcgatccccgtgtgtggggatgggcgccttcgaagctttggtctgtttctgtctctaatatccttggcataaagcttgtccaccataactcgtagctgccacctgcagactcggtcacccaaacttgtgcacactgatggcctagcttcctaatcacacgcatcttcgtttggccccctctagaacactcgctgaggagggaagaggtgggggcagtttgtgtcagagcccagttcaggaaggccttggatttttttaaaggctaaaataatttaggaacagccattttgcaatataattagtgttaccactcagtcactgagaacaaatgtatgacttgaagaattcttcaatagctcgcgtgggttttatagtgagaagaatttctgctaagtttatagtcatatcagtgtgaaagcccaacacttcctctagatgggaaatacaggccttaataaggtagtgtgagagcagttttcagaatcattatgacgaatgatgacttgtgtcctcccaaaatgaccgagtgagttggcatggctcatttgtggaagaagctttcttcatatgcacggacatttcttaatcaatattctcatagcagaaagccgtatttcaaggttcccttcctaagttcttctgtctcattgttctgctttgcgtgactttgctttcatccctcagtaccatcttaactgccctttgacactttaaaatttattataaggcttcaagtcacaatttgacttggccacttttccctgctaaagataagtaagttgtcctgtccagaaaagcagtggctgccaatttctctatctttgaatgactttaattgtattacttgttcccttagcacatacaaaaacccctacaaatatctctgcttgattattggatatttttcattgaaggcaaccatttttatatttctagtactagaatatcctaggtcacttgaatttcaatattcattttctgatctttttagacatgtgtattgtttgatgacctagttccatttagtaattaaaaaagaaaaagaagaaactcctttgctccccgctaccacctccttttcagtggtacctagccttttcctaatagctgctttttttctgcaccactcatttattgttacacttaatataatactttgctatgtaaatggcatgttgatgcatgtgatattaaaactgcattcactggggattaaccattggatatgacatcatcagcagccaatccttgatctgatttgaacatgcattgacaaaagtcatattatcatattgagcaagtgattctatattgcctgtgatgaataagggaaagaaggaagtctttttggcacttactaactctccgttagctgcccagtttgagagttttgggtatttctatactaagaggcttagcttaataataacattctcagattgcaattttatagatctctttagaatttgcataagtagattttcccccttgtgctttcttcctctttgcgagtttttcaccctgtgttctggctctgtttctttaca

SEQ ID NO: 54 - DNA, 5298 bp, Homo sapiens
cttaaaagatacagagaatctgcattttctcccttcctgggcccaaacagccatgcttaaacctgcccagtaacgcttgagaggctgcagccgccaaaggtgggaaagtttcacctctggattagcttctccttttctcacctttctctctctgcccatgaccatcccagggccagacacaaacattgagcccgcagagtcacaagttgtttccccacggatcaggccccagacctcagatgcccctgaagctcaggactgctgttccagctatctggtctcagagtatctgacaatgacagacccatctcaggaggtgtcagggagaccatggacgcaaacctccatctgtcagctccaaccttgggttaactttctcgttggaagagctaactgctttcttactcaagatcctctgctcagagccacctgtgctctatggtgatcccactccttttttttttgagacagagtcttgctctgtcacccatggtccaggctggagtgcagtggcgtgatcttggctcactgcaacctccacatcccgggttcaagtgattctcctgcctcagcctcctgcgtagctgggattacaggcgtgcaccactacgcccagctaagtttttgtatttttagtagagacgaggtctcaccatgttgcccaggctggtctcgaactcctgagctcaggcaatctgcccactttgtccttccaaagtgctaggattataggcatgagccaccatgcccggccccactccttttttgcaaacaccaagtattgcccgtcccaaagcaagtgtccagagtacacatagaagcagagttgtaagtcctagagcctgtgaacaggtcatgagctttccctctacccagctccaaattcttgcccttcctccctctgacctcccttaagtccagagtcacagctaccgccaagcaatgcagcccagtggagagagagaggctttcagagctgggtcccccttctccaatttcttcctcaaaaggtctattttcccacactctttgcagggtacctctgagctcatcccagcaaaagtggcaggagcaaagcccctcaggaaggagctagggggtgggggccagcaccctgacttccagaccgcttccctggcccggggttctatgttattcttcctggattggctgtaggtgatctctaggctctcattttcaggcaggtggccagttggaccatccagcagaagggggagagagtgtggctgttccactggagtcttcttcaggcccctctcccactcctccctgcaatgcggctagaacttgaggaggtagaggtttcaccccaatcatctgggcctcctgccttgaggaccaggccagttcttggcacctactggcctgattccacctgggctgtgctccctgagaatggcattatttctgatactggcttcttcttgtgtgtttctatccccaagaccacagggcctccccaggtgccttcagaaaaccgaagtattcccactgcttctggcccaaaggttcctgcccccaggctcagagtgctagatttagggtcactgtgggcctggttgtcaaatcccacccacttctggtgccccttcaagagctcttggaatctccacctcccttccagcagaccatcaaaaggtcaggctcccccacccaacctggcaccccagattctgtcctttccatccatacccctaccatgactttagggtcatgactcgatttcactcttctgctgttttgcttccagggtgcccaatcctgacacttccgctggaatgagctcgaggtactctgcaaagagcctgggaaaatgaccttccagaggactccaatctttttttttcccgttagccctgtccagttctttctgcgccccagatttgcccctgtctcctcgcctcccttccgtgtgtattccagctcctgtcccagccaccgctgcagacagtggacctcccagattcaaattcccagcccgaccccacgctcaccgccgccgttcttgtagcagaggtaggggaagcgccacacgttggccaggtccactgcgaagccgactacggacagcaggaagtcgatcttcttgccccaggtctcccggggctgcgcgtcgccgtcgcggggcgccagcaggcactggacgccgttgcgctccttcaccaccagcagctccgcagttttgcgcgcccgaaggggctgctctggacccgtgtccgccccgttgttctcgggctgcacctgcgggttcatccgcgccagaagcatggatgcggctggcgagaggaactttaccggtcctgggggcacgccgaggctctgcttggataaagggaaaagaggtggttacccggccgttcctccctacttgcaactcccaagaccacccgggagcgccttagacactgggatgagcgcgcgctgagcggacgcagggttcccagaaaagggcgcgcgtccgctggaaaaatttggaaacagaggttggggcacgccggaactcaagagttggagacttctgacagccctctgggggcggctgaagaccctgcagcacttttcctcgttcacccactctcgcctgccccaaatggctgggattgaactcaccgaggctgagggcagcgtgccgaccccctccccagctcaggtccgcgacacttcaacagccacaggagccttctcggacacttaacgtcactgcactacaaactcgctagccctctgctggcgcgtctcggcccgggcctcccaggcagacctagccctgtcccgcgctcagggcgcactcacgtgtccggcggcgaggggatccctggtgccttgcgccctgccggcccccgaccggcggcgccttggcaggctctagggcgcgggctggggctgggctccgccgtgtggcgttcgggcggggccggggaagagctggaggcgcggggcggccggcagggcgcgggctgactggttctggccgtgcctggcgggggccgggctggtaatgtagcctgcgccccaggtaacgcgccgattgcattaacccagcgcccggcccgctccgtctggagcctgttagcgctaatggagactgacagcgcggccggggatgggcaggggcgggagggtggcgcgggttcccagggatctgggggactcgggcgcgcactccctttctatcctgcgccgagcaccgctcgccgctcccaaggacaccaggcctctgatctcccaccacggggagcgagtgacagactctccagaactggggtctgcgtcgcgtcagaaaggaggccaaaaaaggactggggaagcagcgaccggtgcagcaccagctctgtcttccgcagcaggccacttaaactatctggtcagtttctttatcaacgaaatgggaatgtgcggctcagggcccagtgctgcaaataggcggccagcgggctgttgggcagttttgtttgagcggcttagcatgttgacaggtcacagcccagcacttactcctgcatctctgttatttatgggcagggtagcaggtcatttgaggttgtgacccgtgttctgtgaggctgagaactactatttattctttcaattattcattcattcacaccttcacacatttgttcttccatatgcccgctgtgtgccaggcactgttccgggctcttggattacactggcgcaacataactcggagattcctgacctcatggagtttacattctgctggcggagacagaggaaacgggaaacataataaatacacacgttatatgatgggtgagggaatggggggacagtggggctgcagtttgcatacggggtcagggaatttgagaaggtaacaattagacaagtaaaggagtgagccatttggacctgagagtcgttttctcattcattcactcatctattgattaatgtattaatccctttagcagatgctgatcatctgctgtgtgcgtgaacagtgctggctagagaggagatagcccctgccttcaaggagctcccagaccagtgtatagagtctatcacaacccagtgtggtcaggatgtgttgtgaccacagatgtgtggaagtagggtggagctgaatcagcaagggaagcatcctgttgatccaaggaggcttcagagaggtggccgtgctaagtgggttgggtggggcttggatcagcccagccacactgacctttccattctgtaaacatcctaagctcaaacccaccccaagacctttacacttgctgttttctctgcctgagttgttcaggtctcagtccagaggtcccttcccgacgactcaaatcgaaaatacacgctcactctatcctatcaccctatttattcatttgttacatcgctatccgaatttgtcctagttgtttaactttatgtccccccttgggtggtatgtgagctccatgagcacagggaccttgtctgtcttttacagcacgtgttctggcacctggaacgtactagagcatcaaccagcctatgaagtgaatgagttaatgaatgaaagaatggtggtgtgtggcggtgtttagagcatctgtaccagagggacaatcgtgacaaatgtcctctgggcaggatgcagaggggagaggaagggaaacctagaggggtatgggttcccctggcttctggggaagatgacagaaggcaggtgagggaaaagattgagcagaggtttgaggacaagagctcagaactgtaggtttccacacatgaaaggtgtttatctcctttgatctattggacgatcatgacaatcctgtaaggaaagcaggaagagaatcatctttccacgtatcaggtcaggaaactgtggctctgtggactaaagggacttgtccaaggtcccacagattggaagtcacagagtggaaatgagaacccaagtctcctggcctccagcctctgctccctccaccaagtcatggtggagacactcagaatctctctagtagtcatggtccccttctctcctctaccccacaccccatccaactagttttcaagtgctgagaatcccacctctgaaacaccctgcaaagccacaacagaggaccctcatcacccctcactgcagcagcctcctagctggtcaccccacgttcagtatctttccagctgcatgatcgtcaccagctgccagagctctacctagaactcaagtgcgaccacctcactcctctgcctgaaactcctctgaaacacaccaacacataaatgtgatttaattaactctggatttgaggtaatttt

SEQ ID NO: 55 - DNA, 4723 bp, Homo sapiens
cttcactgactttcagtaagaaaaaaacgtatttaaaattcctaaaaagccatgtcacctctaaaagtgatttcagacatgcagttggagcctaaacatatgcaagcttttaggtgaagcacagatgataagaaagtcaagggagtggactctcttttaagaaaatttggttgtaaaaacaagaagcaaagggtctcagggacatatatggtctatagttattgggatgaagagtcttctctggagaagaggaagaggccaaaggtgtagaaagagggaatcacgtggggatatgtctaatggagcctctgacagtaacatactgtgctttcctttctccagcagcctccatttgggctgggctgctttacctggatgccaacatgcccttttctctttgtgccaagcatatcctgatgctacacagactcagaaaggggggcagaactcaccctggggaagagcctatactctcagcctcttagtgtttgaacctctatactaggtgcggcaggcacttttcatcagcacctttccccagtgaagaaaataggacgcttgaaacccccaagagggtctctcacccccgctcccatagaagactgaccaccatccacaactgcagtcataaagtacacttgaccttgatcgtgaaaaagtgagttttgacaattcacaggacttgaatgctccgtaagagattaggttttcttttcatttattttgagcatattaacagctggtgacctagattaagaaataattcaatatgtgaaaaggcagaccatatcactaaattccttcacagatggaaagtggattcagtgaaaagcctcagaccgaagagtcaagtaattacttggaaggcaggaggaggagtacctacaagtgggtaaaatgaagtgtggaaaaaggatgacaaaacctggcccagatcccaagccagcagatccctggctactttctgcacaagtaacccgggcctggaaggcagcaggcagccttgctaattcagctgcggagggagaggcaggaggaggatcactttagttcaggatgagcttgggtagatattaaaagaaatacaatcctcttactgcatggagagtgtgcatgtgtggagggagggactagaggcagccaccaagcctctgggcctggggacaaaagggatcagtgccccctctgctcagcaactcatggtcagccggaactgggaggcagcctgagattgagattgggtctgaagagggaaattcattgccatcctgagcaacatggcaaatctgacttccagtaaaacaggaaaacgctaaacaccccatatcctagggacggaaaatactgccacgtgaaagagaacacagttcaagtgataccaatcatggggctcattagccggtgttgattttcaagtacaatgctcaagctacaatataaaggaaatgtcctttgctcaggacacaaatgaagacctcagggccgtcatatacagcagtcaccagggaagccaggcaagcctggtgcctggtacaactcccctccacctgctttgggctgtgcttgctacggacctgggtttcattcatgcacgtctctggggaaaaaaaataaattactgtgcgcaatttactgagggacttgcaaaacaacagagaacccaatttgtgcaatctggattttttaaaatgcaataaaaggtccttctttgtgcagaaatatgcagaaaggaactattaggttcaggtcatatttatttttgaccttctaaagaaggacactggagtcaaaagggacacaaacatatagaccaaaattaaaagtgtgtatatctagatctctgttttgaactccagaactcaccagacagcactgtgtgatcacgatcctaccctattggtcctgggagatttttggtggcttctctgttaaatcgttcgtgtctgctgcacttaagtagccacaaaatcccccgcaagaaggaaggctgctgcaatctcccgccccgatctgcgggccgcgtccccgcgagctgcctgcgggcgcgctgggagccggcccggggagatgcgagcgcagggagatgcgctctgctccgggcgcagcgggcaagctgccgcacctcctacttctgttgcctggcttggggccaggagagcggcaacccggggccggctccggagctggacatatccggcgtctccggggccagggtctgcgggtccccggcgctgcgtcctcccgacccgctccccgcggcgcccgggtcgcgccttgcggcagtcaccgagctgagggcagggcgtcccagcgtccctggccccggccccgctccgcagcgcgccgcaccccgaggctcgggccccgcacagctgcagccggtgccctccggccaagcccccgcctggcgtccccggccccagccccggccgcccgccgctgaccttttcctgcgcgcggttgagtcgcttctggacgttcttggcgaagatgcccgtcttgatgtcggccatggctgcgggtccggggagctgcgaagagcagagcgcgcagcggggctggcggcggcgcggaggagcgggaggaggagcgggaggaggaggagagggccgagcaagggaggagcgggagaagcggcggggagccgcggagcgcggaggggtgagggcggggcgcggcggcgggaccagcttaaagcgacagagcgggggcgggatgcgtcgggactggctgtgacatggggagaaggggcgggggaagaaagagaaagaggccaagaaaaagagagggaaaagccagaagccagattgtctcctcgatgatagaagagtttcgagaggagaaagggaaccaagcctagcagaagtccctgggcccaggggcagctcgggggttcttttaatgcagtttgtatttggggagagtgaggggacctgattctttccagcaaaaaaagaaataggggagacaaaaaagggaagaaccctagggatgtggaagccagctggattggaactgctctgaagtggagctgatggggctggtcccttgctgctcccaaaagtttccagctaacagaggcccctccatacaccctgggtgttgcagaagaaggtatggaggtcgccctaccctcaggaggctcacagcccagtgaggggccctggctgggaaaccaacgaagtcagagctgcaggagagaggcgtgtggagagcaaagcggggagagaccatcgctgacacttgtttaaactttaaaacaagagttagcagggcggggaggtccgcgttgagaaaggtcagcttccggaagacaggcgatgtgttttggtcatctccaaaattcctgtgctgggcagaaaccttagaatagactactcttcaagaaatggtgcatggaacagtcaagtcccttatagcttcgcatcaaccatggaagacccagtcccccctaacttgttgcttccttggaaaccttttggcaaccttaacagggactcaccatccctattattcttattaatgcttgggagaagtacgaataatagggataaacctgccctacacctagagctcttgttggctctgaccagtggtcaaactattcatattccattctcactgagagtgtgaggagaagaagtcaggtggtgccctgcaaacagaggtcagaagtggtcactcacagtgaatcctatgagtccaggtgagcatcctactgtacctctgggggaaactgttacattggtagacctcagcgagctgccacctggtgttcactcttgtgtcaccctgtagtcccctcaccttgattctagaatgatgctatggccagctttaatcaataagatgtggtgaaagtgacgccatggtaattccagtcccccccccacccacccaacctagaaaccaggcaagttttgccctctggggattcctgtgacaccatataggaaattcagctaattcagctaccctgctgcaaaaataaaataaaataaaataacaaggagaggccatacagaaagggagaggccctgagattacgcagagagagagagagagagagagagcagccaagatgtcccagctgagcccattctccaatcagttcactggctggctgcagccacacaagcaaccatcagcaagaccagcagaagaaccactcagctaatcccagccagagtgcagaatgtgagcaaacaaaacagtttttatgagtcgatcacttttataatgctttattataaaacagtaggtacctggaacacctggtctatgtgcttggctcttatttccctaggataaatatctgccccagtttccttaggaattaaggaatttccaggacacaggactcttagtgctaacagtgggaaagttctgagcaaatttggatgagtcctacttataacactccaagtaaacagaggtttatttcagagagataaaaaaaaatccaccacttggtcaacattcaatgaaaaaggctatgctcagaaaccaggaatttgagtcacctccctcaaacaggggcctttggggctgtaatggttatactgagtgtcaacttgattgaattgaaggatacaaagtattgatcctgggtatgtctatgagggtgttgccgaaggagattaacatttgagtcagtgggcttagaaaggcagacccacccttaatctgggtgggcaccatctaatcagctgccagcgtgcct

SEQ ID NO: 56 - DNA, 4792 bp, Homo sapiens
tgttctcatccagcgcacttcctgggtttccgttgcaggaagtttggcacctgccgatcggtgacatcgacacccgagctgctgcgggccaggacgaatccctccatccgggagcagtggtggcggtggaacaggtggcctggggtgctggaggcaggtaagcagaggcagttgaacccctcattccccttggactgtcccagagcaaatgtgggaagcctgggtcctagactaagtcagtgtgaccttgaacaaggccccaaatcaactcttcgttgcctcactatcccacctgggggtgcaacaggtggttcaggggctgtcatggaggtgagggcaggcagctccacttttatctcttaaataaacttctgggtcactaagcatttgcaaaggggttctacagccaaaagcgcctgaaaacccccagatcgcaccttcctcctttgagtccttcccgtctctgtcgttcagagggtatggggaagtctcctccaaagggtgctgcaaagctggagttttggggggcaggggcaggatatcactgccaagccaggaatgagccgagcagggactcagacagccttgagtcccagcctcgccctgctgcacagaactctcagcacctccaagccccagcttccgattctgtaaaggatggcactggtacctgttcttgagactagcagagctgtgtgcccagggacacagcggctctggtttgaatttggctctgccatttgcccctagtgtggccttgagcctctcggggcctcagtgtccttgaccgtgaacaggaatcatcagccctggtgctcatggctcttgtgggattaggatggggatgaggatgggtaccagcacctagaaagcccctatcaatggcagctcctgggactgctgctgttactgcagcttggctgagaggatcaaatgggaaaaggggcgacaagagggatgggaggggcgatgatcagtgggtgagggcggaggcagcctcctctgaccctcataaaccagcctccctcagaaacagccccagagccaccctggaggggtgactgtagctgaggggttgcaggacgggccagatcctgtctggccaccccgcagatggcgctgggtccagcgctcctcaagcctcagccactgtggctttaatcaaaagaacaatctgtctgcctctgaatcagacccagattcctttccccacaatgtccctgaggcctccagggacccaggcagtccatcccggccccatcccgggccccagagtccaggctctgcctcccctcccgctcccgtcctaattgctccagctgggagggggagggtcactggatgtggagggaggggttcagccacagggccagccgggccctgccttccagagctcctggagcggcctgtcccccgggacaccgggacccacctgcagagggcttaccgctcaggagagcagaacaaggtgacctgctccactgggggtctccgagacccccaccctcaggaacactcccggaaaaagcccccaacccagcatatattaaccactattaatcacagtcctggctgtaactgtcaccttttatcctcaggccaacctggagaggtaggcagtggctacctccactttacagatgaagacactgaggctcaggaggtggcgtgactgctccgggtctcacgcttagtaaacggccgcagaaccctggtcggtttgacgcaagaccctggcggatcccagagccaaagtaagcgctcacctccgccggacagggtaggtggggttgccaacccaggagagcgagggaagggggacggcagggaccactcggggtccatgaagaacgcgaggggatgggcagcggggagggaaggaaacccaccccgactccccattctcctacccccactccccatccctcccattcccccatcgcccccatccccattcccccccattcccaccccctggcctgtgccaagggggaggggaggcgtcgtccagggaacaaagggagggggaaggggcggcgcggggcgcggccgggcaggttggggggcaggtaggggccgcttccgcggggcgggaatcccgcgccccctccccccgagcctcaccttttatggtaaagcggcgcggggcggcggggcggagggagccgagggggcggggagggcggacgggcggaggggacgggaggcccagcggctccgggctccgctggctcgggcgtcggatcggagccgccggggcgcgggcggcccaggtaagcgggccgggtgggtgggggtcgcgtccctccgtccctgtccccggtgcacacaggtgacagcgccggcagtgctcggccggagcctgctcggtcgccagccgggacccccggcgcggcgcccctggaggggcctgcgggtggggggtgcggtcctcacgccctttgcgtcccagtctcttggtctctctccccggagtcggggagtcggcaggtccggggcgagggctcagtgaggccggcggagggccgagtcccagcccgggagcgggagccggggacccggcgcgcagggcggaagctcccgcacaaaaggccggagtcccgcacatggatcgaattaaagctccggcggcgcccagcggtgaccccggcggccgccctgctcggctgcctgcggagggtcccgcgtttccggagcccgggggcgctccaggccccttttcctagccagggtcaggcaaggggagggacgggtcccggggctgctcctctctgggcctcacgcgcccgtcgggggctgagaaggtgtggagatgggcgatacccaggtgtgtgccctgggatgatcttggcgtcggcccctacccctcacctcccacccaagcctgaggaccccggggagaggctggggccgcgcccagattcctgctcttaatgacgctgtaggttctgtccgggtcctggaggcacccccagccgagcccagcccccgggctgcttctccctctcagctgctgcctaaccagctgtccttctctttgcaccttccaagggcctagaccctgagtcttctttccttatatatcctctcagccgccctagggacacactcattctcactttatgaaaaaacgaggccagagagtttctgtgacctattcggggtcaccccgcaggaaagaagtggggccgcgtgtgaacccagctctctacctcctctgtttccctggggtgggaaagaggttgagggaacccctcccctaagttgcagggtgggaggggttggggcacaccccgccacttctgcctaaatttaattccgtacccaaaggtagactgctttatccggtcattaaagaacaaagctggccgggagcggtggctcacgcctgtaatcccagcactttgggaggccgaggcgggcggattacctgaagtcaggagttggagaccagcctggccaacgtggtgaaaccccgtctctactaaaaatacaaaaattagccgggtgtggtggcgcgcgcctgtaatcccagctattcggcggctgaggcacgagaatcgcttgaacccaggaggcggaggttgcagtgagccgagatcgcaccactgcgcagcctgggccacagaatgagactctgtctcaaaataaataaataaataacaaagcttaagccgggtgtagtggctcaagcctgtaatcccagcactctgggaggccgaggagggcggatcacttgaggtcaggagtttgagaccagcctggtcaacatggtgaaaccctgtctctaccaaaaatacaaaaattagccaggggtggtggcgtgtgcctgtggtcccagctactcaggaggctgaggcaggacaatcgcttgaacctgggaggtggaggtttcagtgagctgagattgcgccactgcactccagcctcggccacagagcaagattctgtctcagaaaaaaaaaaaaaaaaaaaaagaacagagcttagaggtggccccagaggtttttttgtttttttttttctttgtagggaacaaggagtaggaaggaggactgtgggggtgaccccccctgagctgcacattctctgccccaaggctgcagcctctagctggtctgggtggctggctggatccctgcctccttccttcctttggacaggaaatgactttccttccctttgttcccacttttctccacttatctaactggatctgtgattcgctctctgggggaggaagagtgggggcggagggtcccctgggcacagctgactgggagatggccccagggaacttacacacagcctgctcccagctgaatcggtggcagtgggacgggagggaagccggggctccctctcccgatgggattgacagggctttgaaactccccagcttagaccccctggggcctgaggtccctgcagtcactgcttgggaggtataggggctgcccagctgtgaacatctggcctcagtgctggacattttccgagaagccaggcagatccgacttcccggaggagtgtaggaagggtcagggctgggtgggaggaggcagagggccatcccgaagccccacagcagcccctggggaccagcccggaacatgccgaccacaagatttctggtctggattgggccattgtagagtgagaacaggcctgggaacttgggcgactttgtgccacccaaggaaggaaaagcgtgtctggggacacacttttggatgatgttccattagaattccgagtgccatggatggcctgggaaagtcacttaacctct

SEQ ID NO: 57 - DNA, 4493 bp, Homo sapiens
aaggactatgaatactaaatgttaagttctaggcaattatacggggactcagaaggacctggccgctgccttcattgagtttaaagggacaggattgcccttccgtcaagaaagtatgtaagtgttggactgcacaaattaatgtttttcccacaaccgagactttggagattaagaacttatttgaggatttaagaattagggaaataatttggtggaaaccgggaatgagttctattcttaaacagcctttttttttctttttaatgttggatatacggcgaggtagagttggccatatttcagagacttagattgacgtatatgtttctgcattatttttacaacaagtttgtgtatcagagcgggagtgcgggggagggaaagaaaacaaacagtttcagaattgaataggcaagtgactgttttaaagattaagtaataaagatgtcttatctagtgtgacttttaactttctgactactgggtagaaattgtacttgaagccggttacctggatacctggtacagattatttttgtttactttctgaaaaacacaaagcaggccgtgcgcagtggctcactcctgtagagtcccagtactttgggaggccggggcgggcggatcacctgaggtcaggagttcgcgaccagcctggccaacacagtgaaaccccatctctactaaaaataaaaaaattagccagacacggtggctggtacctgtagtcccagctacttgggaggctgaggcaggagtatcgtttgaacctgggaggcggaggttgcagtgagcccagatcacgccactgcactccactccagcctgggcaacagaggaaggggccatctcaaaaaaaaaaagcagttcaattataatggtttcagtgataccacttctaaacttatacttcttttggtatcaaatataatattcaaagtagtaaaactgatgtctgttttttaaaaagattttatgcctttcaacactgaacacatggatttcctgtaaatgatcaatttgggcttcattgactaatggtaataatcctcattaatccagaaaacacttaaatctttctctacgtttatatccccagttgtttgtgtgtgtgtgtttgtttttaagcttgacgtcaggtatagttttgtttgggggaagaaagaaacagacccataagatccttaattaggacattattatctttttttaagttccggagtagatgtgcaggatgtgcatgtttgttacataggtaaacgtgtgctgtggtggcttgctgcacctatcaacccatcacctaagtattaagtccctctgcccccttctatcccctgcccccaggccccaatgtgtgttgttcccctccctgtgtacatgtgttctcatagaacattattttttgattcatttgtcaagttggcctgagctggtttgattaggaggtgaaatgtttatatagctgtccttggtcttcctgtccatcaatcttttccagtttgatcgcagtaaattcatttctgtaaaacttaatatctcccataaaggattcagatttcataataacagattactaaaaatgcaaatgtttagttgttactgtctttggatactttctgataaagttgagctttatcgttaatcagcttttccactgggacatggcagatatggcacagccaagccctcccggagcatagtctaatcatggcaagttttaactattgatgtaagagttttaactattgattttgctttcatcaaaagcaaagccattcacacagtatttaacaaaaaccctcgggctctgctaggttcttgtttactgttaaaacagtttaaaggcagggtcttgatcacttaaaatactcttagaagggaaagtaacattttaaaggattaacagtggaaaaagaatatccctgttcctcttccttcccccaagtcctcaacatgtgaaaaatagagttgataagtaaggagatttaaggcagaacccgaggcctacggagcagttaccttctacggcaatttcaagggtggatggtgcgagcgccgctggggcagctggcgttctggttcttactccgtgggaaaatggccctgagcccgactggcttgaggcttagacaggtgaccccgcgaagcgggtgggcaggcgcggccgaggggcgggaggcgggcagcctccgtgattggccggcggagggggcggtgtagcgacccgaggagggaagaggactccaagcgccatggccgctgccgcccgagcccgggtcgcgtacttgctgaggcaactgcaacgcgcagcgtgagtgcggggccggcgggcgggcaggggaccgcagggtgcccacgcagccccctgggtgtgacccgctcagatcctgcgcagtcttctccgcttactttcaagaattcggagaatttggataccgccctgggctcccaccgcgcgccaggcggaacagcgaggtcacagagtgcgcgatgcagcctctcagggcgcagtgaggcagcctttactctgcttgtctaccgttaaagaaactgaggcccagagaggacctggtcccacatcgtagcaggcagtggagagcttggcacggtggccaagacaaattcatgtatgcaattgacacatatttattgggcatatattatgtgccaaaaactattctaggttctggggatacaaagatgaaaggctctggagcagtttgaggtctagtggggaatgggatgaggggacgaggctaaaaccggctatagcagactgggtgtagaagggaaggatggatgtgagaagtcgcagagtcacacttcctggcatgtacagggagtcagggtattcaatgtcatgacacgcctgcctcccagcagaaatgtgaggtacatcctggtcacctgataaatacttgctgactgtgcaagtgcacaagtctgcctgggaaacatggaaaagtttgagctcacaggtgcagatcagtaggaggaaaggcgagggactggcgaggaacgtcaggcagagagaagagcaacaccagcgatggagatgcagtcccaggacacaaaagacaagtagacctacctttataagaggaggtaaagatggagaagacggctgataaaagaccatgaataagaatatagctaatacgatgagcccggcagtgtcttacacactttgaatggactatcagtataatagtaactctaatcctttcactgaccctatgacacacggtattgtcatcctcatagatacggaaataaaggcatggagaagaatttgtccagtgtcacacagctaagcaaatggcacagtcataatttgaacccagatactctggcttcacagtccttgttcttaggcctgtagtaaggaatttgaattgtgtgctgtagataatggaaagccattgaataatatttgtcaccattaaacactgataccttaaaatgtcccatgacaaaagaattacatggccaaataagtttgagaagctccttactgctgtattgccctcttggagaatcaaaatatacattagacatgcagggctccaagaagctacaaaaaatactaaagctgtgttcaaatttttggttgtaaacctgtgtcatcatagaactctttgtcacacaacaccatttatatcccatggaatcagtgtcacacacagaatatgtttttggaatggttgcatgaaaagggagtatggcataatcagaactgtgttagcacattctggtggaaatgtggagtgaagaggaaggactagaggtcattgtcctgctccagcctgaagataatggcttcatgcacggagaggcagttccttgggtagatagaaatgctgagaggttggttagaatccacaggattttgtatctgaatagtgaagagttgagatttctggcttgggcatcaagtaaacagtgggctcatttaccaaggcagggattgtaggagagttagtgttaggaggaaaatggcaacttcagtctagggcattttgagttgagcatgcttctggtacattcagatggagaccccagtaggtgattgaatgtattgttctggggctcaagagtgagttgggaacacatccatccatgcgtatggaggacagttgaagccctaacaaaactctaacctagaaggaaacagctgagggtgaattcctggggagcagcactgccacagcaggagtagcaagatggtgctttcttctgcacatgcttattattctaatagggtaaacattttaaaagtgggagatttccaatgaaaatcttggaaaattggaaaatggcatcattgggcacatgtttcatctgggcaaagtgggctagggcacggggtgatgtgtggccatttctattggacagagcatgcactctgggcttctgcccaacacattccatagagcaactgcgctgacaactgaatggaggggaaagaatcagcactgaaggagacagag

SEQ ID NO: 58 - DNA, 5143 bp, Homo sapiens
acctggccagatcttccttccttttttttttttcaagacggagtctcactctatcacccaaccaagctggagtgcagtggcacaatctcagctcaccacaacctctgcctccccagttcaagtgattgtcatgcctcagactcctacaggtatggtggcttacgcctgtaatcccagcactttgggaggccaaggtgggtggatcacctgaggtcaggagttcgagtccagcctggccaacatggcaaaaccccgtctctactaaaaatacaaaaattagctgggcatggtggaggctgcctgttatccatatcttcctatttttcctgataaaataaaattcttgtggttttcaaatgttggtaattgatcttaatttgtcaacaatatacattgagtcatacaaaacacacctgaattatattctcagtttatattgaacccacaggccagcaattgacttttactggattccagcaatgaatgtagttacagggccaattcaacctggaaaagatatttgttcatcctttctacttttgctttgaggatgcagtgagctataatcacatcaatgcactccaggctaggcaacggagataggccttgtctctaaaaataacaaagaaaacaaaagaagagagatactgagcaggtagaaggggtactatgatattttggatgtacctgttttttaaaatgattgtctactccaatacacagtgaatccctatgcattataggatgagtgtgctggattccatgacttccttgaagcccatgaattctctgagactgtctccatcatcattgggattgtttggctcatgagcatgaattatagagacttaacagaggtagttcaaccaaggacagagaaaggtctgaaatgcctgttgggtgaggagtggcactgagattagtaaaaaatcactgccttgaccacttcaaaacattataaataaatgtttaaatatttccttaatataatttactaacagataattcgaacgaatgtcaaggaataacacactatcattcagctaccatatatatatagctatatatttcagtttgtgaaataaactagtatttagaacaagcaattgaaagccgcaaataaagatgatgaaacaagttggtaaatactttttctaagcaaactggttgtagatttagcattgtttaaattactgtcaattttttcttatgaggagtgatttcatcatttgtttaaaacctggcttttgacacagtttttctgaagtctttagaaatttgaaaaattatctcgcatgactctgtatttcatgaaataccaatattttaacatttccctgtaaacaataaaacgtactaatatggattatgaatgtgaacaaattgtgaatggactttttaagaaagtagattaattaaatatctgtaaccagaaaatacatttttaaaatgtaggtttgtgtagttttacttctataccttgccacataacattttgtgatagacaaatgctctttgtttagaatttctcttgtagagaaagcttcattcagttgtttctttccctttatttatatgcctgtgtgcatgatgtctaaatcactggtcaggagttccctggggattttcgtatctgactacatggcaagattccagacccaaagtaaacctttcagagagtgggaatgagtgggaggatgtcgccctgggcatggtgcttaggaggccagatttccctaaacctgctctgctgggagccctaggaagcctcagatgcagtctgctgtccaggtatactcacagggtatggggtgggggactgtcaatttctgaatagactttgtctcatccacacgacaacatattttaattttaaaggcgaacgtcaacctatttagaaaactataagtgtgactgatgcattcattatttaataactccggggtgaaacttgaggtgaacggtttcaccctttcatactcaggaatggaactagtcctcggtaccgtgcacagtctgacctcacgctcgcacgctgggctcccggcctcctggcagtgcacttaagtgtaccccgcgtgtgcccggcggactcccgtgcacaccgccgccgtgctgttgagtacgaaagacccccgagaggagctcgcagatgagagcctagctgttgctgtgagactgactgcgggtggcagccggcaagaaggaactgccagtaccgaccctggcgctccccaccaactgcggcgcagcctcctctgcggtgcccgcccttcggcatcactgcagtccctgtagcaaagctaactttaaaaatttaaataaaataataattttttaaaggcccgaaaactccggctcccacccctcccccagcctgcccggcggcggcggcgctcagtgagtgacacggcgagcgtgagccgcggccccagccgggccgagcgcgctgcctgagctgagccgccgtaggtgaggggcccgcgtccccgcccgccctgggcgccgcgcctggcactgatcctgccggtcgcccactgtcgccgccgccgccgcccgcgggcaccatgacagctctgagcgctggggttacaggttggtcaccgccgccggtgcccgggaggctgcggcgcggccggctccgtgccctgcgcccgaggcccgcgatgcgcgctcgttagttccttggccggtccgggccaggacggctcaggggcgctggggctgcgagaggggctgctagaggggctgcgggcgggggccccgcgcgggggaggggtggggaggggcgcctccacccgagagcggggcggtggggtccttgccggcctggccggggcgcgggagccgcgatgcgccctccctattcctgcgcgtgggtgtcgggggcgcctgccgcgggatgcccctgggctggcttgggaagcccagggccggcgacacctcgggcctgccccgcctgggcatctagcaggtgttggggggcaggtggctgaggacgaggccactgggcatcactacagcgctgcacctgccccctcggcaccagggccgctgcctgggtaaacctctgggccaacgcgcgggtggggaggtgggggtgggcagcctggctccagcaggtctccgcgggggctccgcggccttgccccacccccatgctcctgtggattgctccttgctaaatgtcccttatgatattcattggccttgttaagcagccgtcattagggtggctacaccgaacgcaaaacagtgtcctgccaagcaacaccattttgtcactgaatctttaagacaccagcaaggatggcctggctgggatccctctgggggcccagatcgcagtcattttgctgacttaatgttcctctttcaggatggtgtgagaatgctgaactcagacccttagtgcttgaaataccttagaataaatctttcagtttcaggtacaatttttcagacactgtattattaaggatgtgtgaatacacgctcactttcaggcctcagaaggttttactcttaatctggtgttcgtgtggtcatcagctaggcccgactgccctggtggtgaaatgtgggttgaggaccagtgaccttcgtgttagtagttggtggataacacctctaataggggatcaaaaattaacaggtggcgattttgaaatatatatgtatgtatatatatatatatatgtgtgtgatttgtatgttcataactacaactgtgatttgatatggcctgggagaggagcggtttcactatggtagttacccctgaggcagatgcatctcaggactggctttttaaattcaaagataaggtttccacagttctaaataaaaatcatggcttcccaaacatctgcaacgagaaacatttactccctaactggttggctgtcattctgcgtgcttttctagagtcgatttttcgttcttctgtagtatactttgacctagccactgctaaaaccagacagtgtgaagacaaaatccagctttggagaaaaacttaaaaagtgaaaaggttcagttggatcctcttctgtgttcatagaagacataatctgtttgcagaataaatgtgccgtcactgataaggactatgctaaaatctctacagaactctaggagaaagcatgtcttataaaatagtggttgtacacttataactttggataggacttgcaagatattcaaaggagaaatgtacttaaaaaatgtacttaaaaaaagtctactcaaaatagtgtcatttagttgcaatttagtgaactgaagatttgagtagcattttcttcttcttgggtatggcatttctttgaaatggtttccaggtgattttatcattccggcatgctttgtgtgtgcatgtatgtgtatacacaaatgtacacatttgttttatcatttatcgatgatgttcagataatttcagaatttccatagctgtataaatttatcccaggtcagagtcacatgtggatttcaagtttgtgaatgaaaattcacatctacacctctatatcctaagataagctaagcactgggccagaagctgggtggtgagtgagaaaaagagataaactgagagcctaagaatccttcaggaaagacaaatatgtaagcaggtggatatacagtgagatgtcttattttctagttttgctgtggaaactatgatagtgagcttaaacatattaagaatggtccccaaggccacacacggtggctcatgcctataatcccagcactttgggaggccaaggcaggcctattgcttgaacccaagagtccagaccagcctgggcaacatggtgaaaccccgtctctacaaaaaaaaaaaaaattcaccaagtgtagtggtgtacacctgtagccccagttacttgggaggctgaggtggaaggatcgcgtgagcccagagatgtcaaggctgcagcaggctatgatggcaccactgtactctagaatgggcgactgggcacagagtgagactctgtttaaaaataaaaaaagaatgcccctgtcccaaaatgttatcaacattttagagcttcactcatgttatacaattctcgcctgtctcaaggaccagcacagattttcataccttcaaactctatgcaaatgctta

SEQ ID NO: 59 - DNA, 5005 bp, Homo sapiens
tactttcatggatcatgcctttgatgttgtatctaaaatgtcatttacaaacccaaggtcacctaggttttctcctttttatcttctaagagttttatagttttgcattttatatttaggtctatgatctattttgagttaatttttgtgaaaggtgtaaggtctgtatctatgtatccagcttcttttctttttttctctctttctttcttttttttttttgtgattggatgtctagttgttctagcaccatttgttgaaaaggtgatcttttctccattagatttcttttgctcctttgtcaaagatcacttgactctatgtgtgggtctatcttggcctctctattctgttccattaatctatttgtctgctctttcaccaataccacactgtcttgattactgtaactatagtaagtcttgaagttgggcaatatcagtcctcagactttgttcttctcctttaatattgtgttggctattatgggtcttttgccttttcatataaacttgagaatcagtttgtgatattcacaaaataacctcctgggattctgattggaattactttgaatctatagatgaagttgggaagaactgacatcttaacaatattaaatttttccatccatgaacataaaatatctctctatttatttagttctttgatttctttcatcacagttttgtagtttttctcatatagatattgtacatatttattagatttgtacctaaatatttcatttgagtggtgtgctaatataaatagtattgtggttttaatttcaaattcccattgttcgttgctagtatatagaaaagcagttgacttttgtacattaattttgtatccttcaatcttaccataattgcttattagtaacaggagagtttttttgtcaattgaaatttttctacatcaacagtcatgtcagctgcaaagacagttttcttttaccttttccttcacaatacgtatacttttaatttccttttcttgtctaatttcattagctgggactttcagtatgatgctgaaaaggattgctgagtagagacatacttgccctgttccttattttaacaggaaagcttctagtttctcaccattaagcatggtgttagctgtacgcttttgtaaatgttctttttcaagttgagaaagttcccctctattcatagtttgctgctatacaatatgattttaatataaaaaaagcagtacataaagtagtgtgtacaatttcattccgatttttgtgacaactgtattttgcgttggaaaaaagcctggaagagaatacgttcataattgaaagtcgtcatttttggctggataatggatattttcttctccacatacttctatcttttctaaatttgccccgatgaaaaataatattaggaaaggtgattaggtagtctgaagagaatgcctttcggccgtcgggggcagtggggtggtaaggaaaaagatgagaagcttttaaaatggaaatctatgaagagagaactgatttcgtaattgcatcatcagtgggaaagtaagctctctgaggaccaggtctgactgagagatccccacagcgtaatagataatacattttgtgtctcagcgttgcctatgaaacgccatttattcacttggcatgctgagcatggaaccaggtttctttctttccaagtagttttgctaatttagagaaagagagagacagacatatacatagagagagagagagagagagagagagagagagagagagagagtgtgagagagagagagaaaagtactgaactgggagcggcagggtttgagtcaaaagagaagggtggaggagcgcgtccggtgccccaaggcttccaggcggagcgcgggcgcgaggtgcgggctccggggcggagcgcacgcccgggaggcgggaagtgggcagcagcgcccccgcctggcccctgtgcgccccgacccgacgcctccccgcccgaccaggtgcctcgagagcgcacagctgggagggctctcccggtcgcggcgagactcacgcctgcggtgtgctagggcggcggcgacggtgacgggcgtggggcgcggcgctgcctcggctgccgggtcgttgcggcgagcgcgcgggccggcctggagtcaccgggctgaaccgccgcgcctgcatcgtgccgcacgccgcggaggcgctcgggtacagaccgcgcgggcgcgcacaaagcggcccggggcggccggcgcggcgcagaccctcggtgggcagcgcactccagtcttcccaggctagcggctgcagggagctccggcccgcggcccctccgcctcaagtctgggagctgccggtcccactctgtctttgcctatggggattcgagagtttcccagcggcgcacccaggggcaagagcattgccgtgtgagtagcgccccgggcgtgggtgagcgagccggcgggcgggaggccggagcgccggggtagccctttgcagctgggacactgggcagtctgaggctcggggaagggcggcgcgcgcggtggagctgctcgggaagtttcagtcattctttccctcgccactcgccccaacccgcactgccccgagacgcccaggatagggcactggggacagctgcccggggaagtgggtgagtggcggcgggaagggaggagcgcgggctctgccgcggcgctcgcccccggagggccccggccgcgctcttgctctcgccctgggatctgctcctcacgaatcccctcctctctgtccatgctccccctgcccacccacctgcccgtggcgcctgcgaccgcagtggcatgaggaggtcaccggacgtcagcccccggagactgtccgacatcagcccccagctccggcagctcaagtacttggtggtggacgaggcgattaaggaggatctgaaatggtcgcgctccgtggaggatctcaccagcgggccggtggggctcacgtccatcgaggagcggatcctgcgcatcactggctactatggctaccagccctgggcagcgagctgcaaaagtaagaccccctcacgtcatgtttcccgttcactggtccatcttcttaccattgtcacagcagcctcggggagcgcacgcagtgaggcgtccagccaccccctcccgccctgcctgctgatacagacccaggtttgcaacttcgaggaagaggcccggagacggagttctcctggacctctgaggcgttctgatgcgttctcttacaccccttcctctcccacacacaggatctctctgtatccttatctctccactgccaaatacggaggacactcctatgtggcagaccgtgggaaacagatcagaaactcaaagtatcccggacagtgtggcttccccgcggatctgaaagtctcacagggccctcggtcaggtctcaccaggctcagatggagatgctgacttacatagccaggattttaactcttccttgcatctcctagccctgatgatagaggacaattcttggggtcttcattttggatgccgttcacgcccttctcccctggattttatcggtcttcagtaaggaggttctatagggatcattctagatagtggtgttggtccctgcaagttgaatgtttggtgtcaggtggagaagctgaaatgtttttctttccgtgggtcctagctaatggctttgaggatgctgtgatcaattagacagacattatcttatgtcaccaggactaatttggtggacatttctttttctcactttgcaagagagaggttgattatgatgaaatccagatatctataaatataaccataattaggtggtctcatcactgtagtttgacataagagattgccatttgccacctcagtccgtatgagaaatgggccttgacgtctttcatttgtaataaagactagattaatagcttgttgattttgagtttttttcgaccttttattgcttaaaattgaaacattatttattggtctttaaggggaaaacgggcgtttgtaatttaatcatattcatcactaaaaaaaaatgtctatctttagattattcctaaaacctgcgagtccaaggcaaaggtcagtggtatgctagcagttagctggttagtttaaaattatttgcaggaagaaacaggatttgaatgttttgttttcatgcaacttgcttttaaaagactgtgtttcattgggtataagcctttaaaaaaattgtctgtatctcttccaatctcttgcctctgctgctaaaattgtagcttattacaaaaggaagacaaggggccaaattctctttttggttgaaaaaccctgggcatgggataacaaaaattaaaccccattggaaaccatatttcttatgggcatggatagaacgctcaagttttacttctgctttctaaatttctattagaaaatagcaattttctattcttatttccattttctgcttccttaataacaaaagacagtggtataaatgacagccttcataaagtcatctctttggtgcttgaagtcataaaaaggttttatctttataaatctatttgtgactggctgtatgtgctttgtggaagttatcaccaaggtttgtgatttgggggcttgccagagagcttacagcagtgatgactgtttggtacaacatgttttgtaattaagacaagagaagggccatgtggaaatggagtcccttgcatcactggaggtaattcttaaatgcctctctctgaggcttcactggaagagtaaatagtagcagggaaggctacagtgatcattgaaatatgaataaaatcagctcattccactctctctattcctgtgaggattcttctttattccaaggtatttccttgttactataacgctttaggtgacaactgacattttggtgaattaattaggtgtaatgtttaaatttagagaaggtaatgttttattaatgtattaatgcagcaaaagaggttgtctt

SEQ ID NO: 60 - DNA, 4983 bp, Homo sapiens
gttgaaaaaataaaagacgattctgagaagaatagaaataaaacctaaagctctcaaaccagcagtggaatatgtatcactttactaatctgatagaaggcaagaatggaagaaaaagaagcaaggagaaagaatggtaaatgaaaaacacaagatctgatggaagaagagcccaaatatattagtaatcgcaataaatccctgtggattaaattcatctgtcagagacacaaccaaaataaaactcatagaaagattaaatatgaaggacaatatttcacatacaaatagtagccaaaagaatggtggcctattaataacaaaaacttctaatttaagcaattagcataaaaatggtaaacagacgcactacataaggatcaattgcgatttctaatgcagaatatactttaagaaacactgcctactgtgtgccaagaatcatgtatgccttctatactgctgtgttttcagtgcttggcacataataggtgtgccataaatattaatgagtggaatgattgctattttatattgaaatgatctgtttatgtgtttgtcataccacaagggcaagaccatatcttactaattgttatgccatcaacattcagcatagcagctggcacatagtaggaattccataagtccacactgaattcaaggatcatctcttccataaaatctcccctcactgttcctcccatactgatttgtcctttgtttctacttctatagcactcgtaattggaaacacccaatttagcacccaagacatagtatagtcttctagctttcactcaatatttcaaatcttgtttctttaaaactattttagattctttgaagatagagaagatgccttgcactgtgccaagtgccttttcaggtacttgaataaatacatgttttggttgatgtcactatttacatgaatgtaggttagatcaatagtaccagctatacatgtcaagaacgctaagggctatctccagtctcttttttcattccttgaccaaagagagccctgtttggagacaggctcacgtgcaaagaatgtgaagttaatgagacaggtgggagaaggcggctagtgttcaattccaagcccctgtgctctattcctcctggcaaaatagtgaggttggcgggacagggggaggggaatgtctgtgatcccacaaaaaaatgtgtgggaagtgagagggagatggtgaggaaagagagcatgttccagacgctacaggacagacctggacctgacctctccctggcgggcatggcaccaggagtttattatgagaccgaaaggaaaccccaaggcaaacagctaaattcatccacttgaaaccttgtataggagacagtgagggatgttgttttccatctagacaaggcacagaggggagtctgtgacaaggagcttctaattggggagctgcagagggttaacaaagagcactggtggcatctttttgctggtcttcttgaaaaagacatagtctgggaaggttcaatcagagccccctgcacgaggtaaggaggtgggctggctgacttccctaggtccttcctaatcccatagttatttggtgaatcgtgctcttccccatttatgctcctacacacgtctgcacccagtttgatagtaagtcccctaggctgcattccaggtttagtacggtgccacgcaagctgctggcactcaacaaatgacgcaaatatttgttgaatgaaggaataaatggatcaagtgtttaaatagagcccacatgacctccccagctgtaaagtatggcagttgaatgaaatcacaacattctaggcacttgagcaaagctggcggggacacacacacacacacgattcacattcccaccttggaaaagcacagaccacctgcccagccagcaggctgtgggagtttccttggcaaggatcctctttctctgctgctttaggatggtgcctctagaaggtcaagtcacccagatttaggaccgcttcccttcccggttggcctccgagacaaagtttctgtctggatttggcccgggcctcccgggaggcgcatccgcactgactcccggctcgggcgccccgggcgtgcagctcgctggcccgctgcgccccaccttaccttggtgagctgggcgatcttcttgcacattttcacgtgcatctggggatcacagtccattctccagccggcgccaccgtcggctttggagccctggcaagtgccgggcgggttaattttgctgttgagagcagaagccatcgctaaaacgcgcccagcactcagactctctcgttttctccctgcccaccgtgtgcacgtgcgtgcgcgcgcgggcgtgcgagcgtgtgggtttctcgggagaggtggcactgcagtcccgtcgcctgcaccgccgcgtggccccagcttcccgaaggtctccgcctcccgggcccacccgcgcgcccaccctttttcctctcctgctggttctctggctggctggctccgcgggcacccgcaccctgcggcgaggcgtcggcgccccgcacgtgccgctggcgatcagtctgcagttccccagccagtgcaggtttcgtgtgcaaggggagagcttccgcggctaggagctgccggccttggggggtttgcggtgggcgggcactgcgctcttggacacgtgggtggcgcgcggccctggacttggtggcgacggaccctgcccgaggtggggatcccaggctaaagaggaagaagagcctccggagagggacagggcacagtgactggcggcctccggagagggacagggcacagtgactggcggctcccgagtctccgagctgatagcggagatggcggtgggggacgagatgctaagagaggggcgggtaggtgagggcgagccatcgggactcaaggaggtgtgggatccggggcgcacggtgtgggccagcgcctgagttgcgcgagtgcacagcggggctggggatggtagccttcctttttggatcttggagaggagccaggacattgcgggaggaataccggagatgcccaaagaggagaagccaaggggatggcagggtgcgggtgaccgaaatctgagatgagaaagtgcgaaacgggtaatgcgggaagcggtaggaacgcaaagacaaagtcgttagggaggtagaaaggatggtggcaggtgcgggaaaggaagaagcaaagaggaatggagcagttagatgagtaaaagatggaactgaaaatcttagcaaaccgagcccattgaaagtaaagtgagtcgtccaaatccacactactggccttgggcagactggagccagactgtcaagttttcggattctgagctggtgtctgtttagtgtttggtgaggggagaggttagcgggccaaatttctttctggcgtgttctctggtttcagacagtggactttaagggaaagtgacagcaccgtgatcagcttttcctaaagctcgaataataggtcttcatgatagtttgcatagtgaatgtatatcatttatacattgccttttatcagaaattttaataatcttcataaaccaagcaagcttcaaagggtctcagaaaccacagttccacccatccatactgaaggtaacaaaacccatttttgtcgatgaggaaaattgacccagaaaatttgtaccaagtcaggcaattacaagtagaactgtttctgaaatagacattcctattctagattctgtatcaaatgaccggcctcggtttttgatttctaatttaccatcttaagttctctttttagcacaacccctccctcccccagttcctttagaaaacagagacaaagcctctgaggatgggggtcagggggtcaggaatccacattaaacagctctctgcttaattccaaattgtgtatttagaatattttctgcaggatcttaatatatactaaattttacaggaattcaaccactgtgtaaactagaggaaaattagcctttttggtcaagaactttactaaaagttgttgaccattttggaaaatcacatcaagggccaggtggctcatgatatttggttgccaatacaacacacttatttatttattttttttatttttttgagacagagttttgctctgtcacccaggctggagtgcagtggcatgatctcggcttactgcaacttctacttcctgagtttaagccattcttgtgcctcagcctccagagtagctgggataacaggcatgcaccaccacgcccgactaatttttgcatttttagtagagatggggtttctccatgttggccaagttggtctcaaattcctggccttgagtgatcctcccacctaggcctcccaaagtactggaattacaggtgtgagccaccgtgcctggccacaacacacttctaactgccatattgatggtgagtctacctatgcaagcatctgattactacactgtgtccctcagtatcaacaggggattggttccagtaccccttcagataccaaaatccatggatgttcaagtcacttacataaaatggtgtaacgtttgcatataacctactcacatcctctcgtatgcattaaatcatctctatattacttgtaatacctaatacaacgtaaatgttatgtaattagttgttacactgtattgtttagggaataaaaaaataggctgtatatgtatacagtacagatgcaaccatcaattttttttcctcaaatgtttttgacttgtggttagttgactttgaggatgtggaacacattgatatggagggccaactgtacatattatttaattataatgaaatatcaagaatcaaatgtcaaaaaaatcaaagtcaagaaattatgcagatttaaaaaggtgtgtattatctatgaatttcaagatagtaacgtggatgttataaaaagggtgatgttggatctgaaaagaattgaaagccattgctc

SEQ ID NO: 61 - DNA, 4781 bp, Homo sapiens
accttgcccggctgacactattattttcttcattttagagtgagaaaactgagggaggttattagtcctcaaacatacagcttgttcacagttggaactggaattccaacccaggctgcagaatccaaaccagcatgctcaggtagctttagaagtcatcatatagaagacaaaatttggagttggacaagtattagtgattcaaagttgtgggtggtttctggaaagagtgttagctacagaaaaagagaaagaagtagttggtcagtaagttggaagtttgctaggagaagtctgcagaagagggagaaggtggcacgcgacagaatcgaatgattgaatctgatagaatgggcagcgagaagacttgttatgaggaagctggcaagtgggaagaatgcattggctgtaaaagtatgcagacaaaattcaataaacatttacactgcacatgatcccatcctctctgactctctctcctgttaccctttccttctctcaatgtacttcagccctgtcaacctcctggctgccctgcaaacatgctattcttatgttagctcttgctcttctctctgcttggaagttctttttcccttgagatgcccatggctagctcctcgctttcatcaggattccaattaaatgttgcctctttcataaagcccttatggaccccctatataaaagtgatctccctagcagatcacttctgattccttgatttattgtcagtctctctgccttctagatagtcagccaataactgagggtctttgttgtattcactgtggtatcccagtgcctataattgtgtcttgtacacagtagttactagatgtttgctgaataatgaaaaaacaaatgtttagtgaagaccaactatgtgcaaggaacttttctaattgctttgtcaatataacattgttctcaacttcaagaatcagggttgcctgtctttatacagtgattgctacatgccagtcacggctctaagccctttgcatctattgattttcttaaatcctcacaacaaaacaataatgtaggtaccagttatagctgatatttgtaccaggccacaggattatgaagtagtggaactaggatttgaacccagacaatggggtgtcagcatctgtctccttaacaatgaactaaaagagtaaaaggctctcaagaaatatacaaatacatgctacaagggctcaaaggactaagtaaatcgctcgtgcgtttgggggtggggagaagggcaataaggtgtcaattgagacctacttggagaaagggggaagatttcacttggtgacagtgtgtataaataatccagcagccatagggaaggcaaatagtttcacaaacacttagaaatggcttccacccaagtcatggccaactgaccttgcagctgagagtgagagggaattttaattcagtttgcctggagaacgggtcaacttgacctttcttctgaaaagagaggcttcatgcacttgactcttggggacacagaaatctgcattttaattttgacttctcaaacagtaaccatggccacaagtggtctattggtttctctttaccttcccaactattcccggccttactgtgctttagaagggcaaatgaccttgcgggtggtgagcgatacctcctggttgacctgcctgggagatctcaaatgaaaccccccacatggatgcttctccaaaatgccaccagtctatgccaccccctctctccaaggtggtattctgcacctcggccctgcgatgttcacctggagtctagagccagtgtttccccacacttagccgagtcgcatgaccggttctagcttcaatgggggaaagtttcccgtctaggagttctggcgctctgcccggctccggagaagggcatctccttgcgcttcctctctggtgtccttcgaactcagcctaactttgtgcagcagtatttccagtctagctgtccaagttggagcccctgggggacgcagcggcgcgttgagaaggcgcggcgccttcgagcccctccccagagcaggcgctgctcgcagtgacaaggggtcctagaggcgcccgctgagagccgctggcaactcccgggggtgtcctgcccagcccgaagtcacctcgggtctctttcgggtccatctactctgtcgccagaggcaccgtgccacccgcggcgctttcctctccccgcaccccgccccccgcatccccagtccggtgacttggcagcaggtggcaggggcggcgctcacgagggcgaggggctggggtccccgcactcgagccgcccgtgcagcccgcccgcttccagccgccggcgcgaagctgccggcgcctcggcctcccccgccaccccgcgcgcccccgccgccaagggccgcgcagcgcccccggcgccccccgccggcctccgcccgcacctgcagcccgctccgcgcgccctgcccgcagccgccgccgaagctctcgctcccagcgccacggacgcccccgggccgagcggcggtgcgctcccgtgcagacccgtggacagacgcccctggccggtggactctcgagtctcgcttctgcaccctgcgtccctagacatgaatgtgaggagggtggaaagcatttcggctcagctggaggaggccagctctacaggcggtaggatgcaataagcttgcgagcgggcgcccgcttccgccctccttccctcgcgagccaagacggctgccccttcccttcctcggctggggacgcttgttgccttcgctgcatgagacacagtcacctggcgggactgggaaggggaggtgggagcggcaggaatggggtgcaaaggagaggggggtgcatgcaagatccaggtcggggaagaaaagatgtttgaagtgtgtttgaaactcgccggtggcagggtcccctgcgatcctgtgcgtacttttgccttctctgaaaatttccgaggttgtttatgcatctgccagatgactccagaccgcacgcttccttcccctaacacctggggattaatgagctgaaatagtattgatcctcgccttccacttttccttcatgcccccacacgtttcttcattacaactgtatttttgtcccctggtaatatactggcggcatcttgcccatgtcagattctaaagcaggagcgtatggcttgttttatggctagcttcagtttagtgtgttcagagaaaatggtcctcagtgacacttgcaaataatttatatacagtacagccttgttaaggtatcgggcactgtgttaggagcttggctttcagagaaacgtgcgttctgcgaaagcagggagaaatgttttgctgtttggtgggatccatagagaaataggagacgatgcccttccacaaagcgtcttgggttgcatcgatttgttattaggtggttttgatgtgtacagagtattttacaggagccggaaagagaatggaagtgattttggggcacatgagagtgataaagggctggacctctccctgattacataagctaaggggtcacatatggctatagcatagtttgggtttagagggtgaaaaacttggactcgagggctgtaaacttgcaagcaaagaggtgtttccacttgctgccttctttagtactcagggaaatggagtcacctcaacaacttgagtcaatcattcgaatttatggccatgagaaatggaaatggacaccctccctttcttttcctcactccaatagttaaggtcatcgtaaccgggaggacactagtctcacttgaatatgaaattttaaagagaacagtcttaatccttttggactggaaaaaataatatgaacatcacctgtaaagccccacatttgacttgtaactgtgtttagttctgttttcacttatgcctggagatgcccactcattacgagccaagcataagtagattatctcttgtgagaaatgcttacaatctttaagagatcaaatgtttcctgagtggcgtgtttaatgtagagggctgagtagttttggggtgttgagttgagttcgtagggtctctgtctttgcagttctttagaataaacagggtagagggctagcgctgtcctattagagacacattcacacacggcagcccagggaactcaaggctggagcagatggcaggaatgtcgtctcaggatctggatggcttgcattgcttggctcctcagtgaccacagctctttgactgtacctgttcttccatcaaccggatggggattgaatcagcatgtctagactatgcacagggtcatgggtggccataatcactataaatgggaaatgaagatgaagtcacacctggggagtactatacatttaaaaaagcaaaccttttactgctatctccagaatcagcatgtgttcactgtttatttagagaacagaaatatgttgttttataccttggctaaggtgagaccaaagtgatgagttttctggctgagaattgatttgacacctgtttcttgaattgtattcacaaatttttagttagttgtcatatgatgagaagaataaagaaaaaaatagtttggattatcttttttattcttgttgatattgcttcaaagctaatattagagaatgatccccaaatatgtaatactattttcaccaaacatatgcataagtactttgcttacttgtgatcagatataggatggtctttactaatcctgggatgagaagggatgatttttaaaggcttaaactctgtcttccatttt

SEQ ID NO: 62 - DNA, 5529 bp, Homo sapiens
tttacaccttataatcctgtttgttaaaacccataacaattaggtggacatctgaacatatttaaagtattttatccattggatttttatggtccaaaatcaagattctaggacaagataaatgggaaaggaggacatgtagtttgaaatgtgttatatcagcaccatcaatttttaattcggggttccaaaattatctttttctccattggaaaggaatacgtttgtttccacgtaggaaactacttccacttaacacacagaaggtaacttcattggtaggtatgctgcctgtatgcaaggatctgcagttactcataactggctgtgtttttaaacagaataatttttgtcaaatattttgatacaaaacaatgaagcctgaagttaaaataagaaattctgggctcaagtgcagggcaactggacacccttatgagatgagcctttgtgcccttcctgtaagtgtggattagattatccaccacctagatttaagaaaggattaaatgaaagaatatatgtgaaagtactgtgttgactgtattaaaaagaactggaaacccataaaggaccgctataattgtgtaattgttgcgccaggttattttgtcaatacatttttaatcagcacctaaaatatttcatgtcacttcatccagcagagacattgagatgtggcaaaatgtttaaaaggacaacactataggtgaaaaaaaaacagagcacaacagtagtcaattcaaaaaaaattcttttaacaattttattccatttccttgccattttcagagccctttcttttggtgttttgtaacaatataaaaaacgatctggcttttcaaaatatactccttttcctttctatatgcaaatgatatgaaacactggcaagttgtccttaaaagtcagcccaccttcatggcccagataaagtgggcaataattcctctctaggtcctccgcacattaggtgccagtctctcaagtctcttcctagacaatttctagcagccaggaaacataatttttgaccttgaagactttagcattccaaatctaagaatacaatcaggtgtgttttcttttggcaaactgtgagcttacacacacccccagagtggcatttgaatgctgtgcacagtcttgactgtgttcgaggttagagaactaaagacaagattctttctctcttgtaatgaagaaacaaaaaccacatagcctatattccacaaaagaataccagcataatatatccttttttaaagatgtaattcattagatgtgtgtagagaataaaagtgtaaatataggctgggaacggtggctcacgtctataatcccagcattttgggaggccgagacaggtggatcacctgaggtcaggaattcgagaccagcctgggcaacatggtgaaacccccgtctctactaaaaatacaaaaaaaatcatccgggcgtggtggcacgcgcctgtaatcccagctactcgggaggctgaggcaggaagatcgcttgaaccggggaggaagaggttgcagtgagccgagatcgcgccattgtactccagcctggtcaacaagagcgaaactccgtttcaaaaaaaaaaaaaaaaagtgtaaatatagtactagcttcattgatatgtcttgctgcttctgggactttagagaagcgtataagaagaaagtgtgtgtgcctttacgtaagggagcatatgagagagagagagagagtgtgtgtgtgtgtgtgtgtgtgtgtgtgtgtgtgtgtgtgtgtcagagagaatgttcatggcaggaggaggggcggtgaatatatgaagggaagttgggaaaacccttccctttttgtgcactcccagggcagcagccgcaaccccagaatgaggagaggacttaagagcaggcacaaggttcgctgccatgactgatgtgaagcgacctgacgcacaagcggagacttttctccttggcagccaacaacctgcagcaggcaaggcgggcgggggaggaaaccccgaggaggcgctcccggccgcctctttgctgagatagcaggagcagcagcctgaggagccacgagctcagcgtcactgagctagtgcgggtgcaggggcgctcaggcaggcgcacaggccgggagccgcttctgccgctcaggcggccgcgctgcagaaagccgcgccgcgctgcccgagcgcccggggctggcccgcgagcgggaggagccggcagccgcgctccgccccgcgctgagattcgtgccacctgcctcgaacaccacggaggcgagaggaacccagagagctcggcctctcgtcccgttctcgaaaagcagcctatcgtgacggagccgggagaaggctagcgtttcaatcctgtgcaggactgggatttttcttctcataatttgggggagccggagtagaggtcagagggacaggcgaggatctcaaaaagagaccgagggttgtgaacatggttaccccctcctatcattggaaaatctccttgacatttttcacactttgaagcagctgcagctggtgtcgtcggaaaagggggtgggaaaaaagagagggggcggggagagcggtgagacacagccaagagccatgagcggcgcctcctcgcctgccaatcctggcacctgtgcgccgagccactgcaccgaggaggaccctactccgggtgaacaccgggcttgaagccacggacacccagccccagtgcagcgggttctcgccggcgggacctgcagccgcccactcgggccaccgccaccgcctcctgcagttgcggagcgctcagcccctccgagacgcccgctgcgccctggcccctggggatcctcgccctgcacaggcgcggcaacgggcaggggttgttacggagttgggtgcaggattcgcctttcctgcttgtctcttctccgcccaggatttattgtctgtggagccttctgggggcggggagggagctgcgcctccgccaccgccgccgccgccgccgctgctgctgcggtcgctagcgcggcgcgctgggcaggctgaggctggggagtggacgcgctcctgcctcccccgccggcgcttcgggcttcccctcagccgcttcccgcgggaacctctccagccgtccacctgaagggcactggcatcatggtctaacgtggcaccttcctggattcccctctatctcctgttcttcctaccttctactccttccctcctcttctcctccgaagaccctggcgcccactccactgcggacccctgaacacttaaggaataacccttggcagctgcacgactacgctctccgaaacctcatgggcagtttctcccgccggcgatgtgagtaaggcacgctcgctccgtggcggagggtgctgcgggggcccggccgcggcgaccgtaggcgcgcgaggtgtgggaccccagaggccggccgggggcgggggtggcagggcgggaggcttcggcgctcgcggggagctgcttgtgcggtggccgagggaggcggcgaagatgtggtaggttgcagagcatttggaaatggggtgctaactgggcgggtgcatgggagcaccgagtagcttgagctggcaagagaccttaaagggttacatactgcgaaaaggaaaggtaaatgttgggcgctggtgctgggaccattggttccgctgaaattaggtttagctcctcgttattatcttgggtatctccagtcagcttttttcccagaggtgtctgagggatgaagcgcgtgtttgatcgtgacacacacacacacacactcacacacacttgtggcaggacgcacttgtctctttatcttggaaggtgttgtatcaatgtctcgatgtctgtttgaaaaaatacaattagaagatttttatgtgattgggattgaagggaaagggtggggtttggggggatggaggggacgaaggaacatgaaagactggaggacggaaagaagtgggggagcgtgttccctgggagtcagggagaaccttgcctccctccctgtcagctagacaactcctgcccacacatctcctttgtgtggcacgagaagctggtgaacctgcctattcccaatccagcgctgggtccccttgaggatcttgcgtcctcctctccgagttattaaggatgcaggacgtgtttcggattcgacgtggaaagagggagggaagcacattagcattaaggtaatcatggcaccctggctcgtggactattagattggctaccagtggtctctaatcttactgtttgcagagtcaagaatgggggcgggggttgggggactgtatagattgccttttaaaatagtgtgacttcataataacggagatggctgaaagattgcttccttacactacttctcggccatcagggctgaataaaggacccgcctccgccctctactccggctcttctctggctgtctcttccccatcaagtcataaagcgagcgggttgggcgagagggcacagctcgaggcagagaagagacggggtcactttcacaactcccaggaatcggcttcagaaaggaaccatctgcgactcccacgttcctttcgaagttctccttcaaaaggaattacaaaaaaaaaaaaaaaaaaaaaagcgatgggggtggggatcggtaagacacaggggtcctccccctgctcttttcaattactttcccaatactctttcaacacatcgaatttacctgcagtggtaactgggtcagcaggtaaatagatttgtatctgtatatgtcgaaatgtggctctcctggtggatgaaaagcaattttttgtgagcatattctaacctaggcgaaatttctaagcacaatagagatttttatagcgtgggcgagaagagtgcattccagcgaaagcaggacacgtgatccctccaacgggtgagatacagtattaggaactcgattttctttgttccagcctgagcctatgagatgttcagcatcctggttgcttaagtcctcctatagaaggaaaataataataataataataataaagttgatcggaggcgaaatagagggaaaataagcacaggtagacatgcgtgaacggtttgaaagcttcgcctctgcctttttttaccgcccacccttcacccccaccccttgctccagccagccccctattttaaaatgaggcatttcctctgggagacggtggaaaagttctccagcccctgcacacgtgggttctccagcacttcagaacctgctgtgattcgcaacagattacaccaccttcccagtccaagtgtcacgcctccgtccaggcttccttgaattggcttccaggaaggactctaagggaactaccacgaggtgttttttgtttgtttgttgttttttgtttgtttgtttttacaaaagacagtaaagcaatgaagtctttactttttagaaatcattgattgcacagggggaaaaatgagcaaatagggatgtaggaatgattttatgtttccttaagagtcat

SEQ ID NO: 63 - DNA, 5062 bp, Homo sapiens
tttcagcattaagagagaaactggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggtgacagagcgagactccatctcaaaaaaaaaaaaaaaaaaaaaaaaaaaaaaaaaaaaaaaagagaaactgttgtcaacaaaaaaatcatgtaattcccaatatgtatatcacaataccttgaaactttgccatgtcacttgggtcccttgtgggctctgagggagcagctctgagactgatgcaacaaggttgggtgttgatggaagggatgggctggcttgggagcctggctgggagctcagaggtataggaggaagtgtctcttgtcagggaggaagtaggagtttaaggagagtggtgaaggttaggggtggaagagggtgatgtgcaggtgacaggtgaggacacctcttggagaaactttggatatgtcacagtgtgggccaaggcttaaacgttatgcactcagagctggagaagccttcaagaagaactaggatgaaggattaggagaactggcatttgaaaagtggagtggccctggagcaggccatacctcctcgtagctcattttcctcatgtctaaaatgagccaaatggtcttttagaggctaggtgcggtggctcacacctgtaatcccagcactttgggaggccgaggagggaggatcacttgagcccaggagtttgagaccagcttgggcaacatagtgagaccctgcctctacaaagaagtggtgcgagactgtagtcccaggagctaaggaggctaagctgggaggatggcttggcgtgggaaagtggggactgcagtgagccctcatctctccactgcctctagcctggtttgcagagtgagaccctgtcttaaaaaaataaaaataaaaataggctgggcgcggtggctcacgcctgtaatcccagcactttgggaggccgaggcgggcggatcacgaggtcaggagatcaagaccacggtgaaaccccgtctctactaaaaatacaaaaaattagcccggcgcggtggcgggcacctgtagtcccagctactctggaggctgaggcagaatggcgtgaaccccgggaggtggagcttgcagtgagccaagattgcaccactgcactccagcctgggtgacagagcgagactctgtctcaaaaaaaaaaaaaaataaaaataataaataaataaataaataaataaataaaataaacccctcaaatggcaacagcaaaaacacataaatggtctcttaaattccctggaagttgtaaagacagtaagtacatgccggtacctccctcagttgcttgagctgttctcctggaactcactgcccttcagatagccacaacacttgacacacaatattttccatacccgtaggcatctatcattcagaggaagaggcttatcttccagtctttgtaccctgcagggcccaatgcaggctggctggtgggcacctggcctccaggaatgcttcctggaatagcaatgaatggcgctctagtctcttagagtctcagcatttatgtccatttcaattaaagaaaaaaaaaaaaaaagaatcgcaggaggtggtgtagtgagcagcacaggataacgcgcttcgtaggtatgaactcagttaaccccacagtcacactatgaagtgtgttatccccatttcccagataagggaaaagacagagaggttaagtaatttgaccaaagtcaacagctggtgactggagagccaggtttaaaaaccggatgccggctccgtgcagcagcccgccgatggctactggagcggagaggttccgaggtttcaggggcattttgtcctttcccacttctgggccgtgggctcccccatagacgatcccgcgggcccttcctcctctgacaccctcggatctggttccacctttcccggaccgcagctcagccccgcgcctctccagctcttcgatgaccggttcccggccgtgccccgggccctccctcggagcgcgcgccgccaggggcacctgtccctgcgcgggggtgtcgccgcccctcggcgccgccgggcgcttgccgctgagccgtggcgcccctggcaggagcactgcaaggacgcctcccgggactgcaccgcggccctgcgcacttggggcgacggggccaggcgcgggggacggctgcgggctgggagggcgcgcggaggagacaccgctgaggggacgccgctgagggcgccacgcggggggcgcggctgaggcgcctcgaagggcaagcgccaagggggcgcgggcgccgccggaagctggggcggggcgcccagcgggatgcggtgaagggcgagcggcgcggcggctgcgatgagtgcctctgcggccaccggggtcttcgtgctgtccctctcggccatcccggtcacctatgtcttcaaccacctggcggcccagcatgagtgagtgagccggcgcggcgggggtcgcgccgaggggcggcgggagttggctcgccgcgacgggagcctcgcaacttttccgagggggctgggaccgtccgccgcgggacagaggttcgtggccgcaggggctccccgcgcctggccagactaggggggcgccccaggggtcgcacgggccgggtcttggagccgggccctgaggtgcccaggctggcgcatttcgggatgttggtgccagcaccgcgccaggtgcccggggtcacaggcgtcaatacggccatgcccctgccctggaggggcccaggggccggtggggagcggaacaaacacggacactacgaacctggccgtgtcagctcgtttgtgccttgttctttttgcatctcacgggaacgtttccatgtatggactttcgcatacttatcatttcttattaattttagaaactgttgtgaatgactagcagaaatagcccggaaccaacaggcagagacgtgggttctaggcctggctttggcactgacggtgtctggcctgacggtgtttggcactgacggtgtggtctggcctcggttttccggtcaagtggactttgtgtgcgtcctattgtaaggttccaccgagacctctttgtgaaggggcgttgtgaatggagaagtgcttcccagtgcctgcgctggggtaggatggggacaggcagcagaggcagaacacttcaatgtgaaggaaatgggtggggctgatggagggggacagggcttgggttcgaagccctgcctcccactccactgcagggatcacggactaacctgcagccggcagctgaaggcctgagggtcgccgggtgttgggacaggcgccagcctctgttcctgctgatgttggttgctgtctggagggaggaggaagtaacctgctatgtctctcagctgttggtgctaaggaaattgcatcagtctgtaggctgcaaacccccttcagctggtcaggccgtggagtgtctgtacatagggtggtaacttcctcctctccagctgcctggccgcctgatacaggcagcacccacactgagtgctcaccaggcaccagcagtgtctacccttggtctggcccagtctctggaggcatcccctggaggcagctgctctgggccaccgaatattacatgcctgtcaatatagtgcatgggggaccctgtgctccaaaaaatggcattacgggtagttcagtgaagatatactgagtgcctactgtgtgtttggcactgttccaagtgttggtgacatagctatgaacaagccagaagaggctgtggaggcaggtaatgagatagacatcaacaaacattaatacatcgatagtgacatgctatgaagataataaaatagggtgactggggagggggctggtggccagggaaggcttctaggaggaggtaacacttgagctatgggaagatctgggaatggagcctgtcgggcaggaaaatggagcagtgcaggggaaggaattggatgggcgtagtaggtattttggaaggtgaacagatggggcttactgaagagggttgcatggcagcagaggatttgaggatgagccctaggttttggcctcagccactgggaagatggtgatggcatttactgaatacagactgaactatgtattgctgtgcatgatggagcgcactggcaagcactacagcattttgaataattagtgtcagccattcttcctgtcctcttgcaggttaaaacccccgtggcctttgattcctctcttctttctccactcccttcctcctactgtctgcttccccataagtcctggggctctagtgtctgttccctcctttcagttctccctggcactggccctttctgacctggactgtggcaaaggctccggtgcaagtctcgctgcccagccccgctgcgtgtttctcggcctcggccccccagtcccagcccacgcactagtgctcctgccgcaccagcctgcctttgctccttgaccattcctcctcctctccagcctctagcccggactcctgctgctccctgacagggatgctgccttcctcctatcgcagactgttgaagccgcacctggactttatggccacttctaccttctgcagaccctccctctctctcctttgagctccctcagtcccttgtttgcacctttctcccgagagttactttcctgtggtaagaacttacctgccccggctatagctcacccctctgtactgtcctgcccaggccagcgccgtgtgcatcgtaggcactcaggtatgtttgactttgttgaatcgagtttccttcccaccaaaccttttatgcaactctgaaaaccagaaaaagaaaattagattgagaggtcaggaaag

SEQ ID NO: 64 - DNA, 5479 bp, Homo sapiens
ctttctttgcctgtaaaataggaaatatagcatcaaccactttgggatgttgtaagtaatagattaagaaagtgtgtttagcacctcgtacattgtagtagaaacataacagaaacagcaaaaacacatattggttgagcccatactatgtggcagcactattacaagcacttaaatatattgctttatgtaatctttgtaacagcccaaagatacacgttatctttgagataagatgagaaaccctaggcacaaaaatgttaacttgcacaaggttggttgcatagtaactagtctgtctaaatcttgaatgtcttactgtctactttatccaaaggacactttttagttttttctctttaaactatatttgaaagtttacagttgatgttgctcgctcctctaccaaacactcctcctcccgtgatccctatgactcccctctgtacaatctttcctcctgttctcattgctaccttccaatctcctctttcatttcttcaactcttctttcttgaaggaaccatcccatgtggcaactcagtttctacctttccaatgatgattgccagtcttcactctctcctaaactctaggcttgcatttctaagtgttattagggtgtgagcacatatgacccaggtaacgttttgaaacgtttgtgcctttcacttctttcttattccattggttgcaatcttgtcacttaatacttgaaatgtttttcaaatttggatgtttctagcaatcagctaatgcctttggcctggtttaggccttcatcatctcttgcctggtttatcaaagtcattttttaactgaacttgttgctttcagttcctctctgacatgatggcattagggtaatatttccatgtgcttcaacacacataccttctcaccattactatatgtcccccccgccacccccccccagcatcacactatctctggtgtcaccttctctgttcatggcctttttcttcttctctaggggttcccccctttgaagaaacagcatctgctcaacctcccctacaggctcccttaaagctggtttctcctccctctgtttgtctctttgcaactctctggaggcatctttacctttacaactttgcattttctctgctattcccatgtaagttccctgaaaccctagtaccattcacagcaggatttcaaaggttgcctgttgcgtgagcattctataggatcccaaattatctttctgtgcaaatatgaattgttgtgatatgtccgtagagacggcacaacctcatggctaagaatgatagttaccagttctagagatcctactctaattgctctagagtgggggcctgaacaccagtattacaaaaaagacaaaagaaaaagaaggaaaaaagaaaaaagaaagagagaaaatcaaagctaccttcatcatgtatgaatttgagcaagtaacacctttgtgagtccccaggccccagtttctcattggtaaaatgaggataacaattacacagaccttgttgggctatgtacagaattaaatgagattatgtacctaaaaggcttgtggcacagtgacacacacacacacacacacacacacacacacacccagagtacttcgtggttatttctaggctttactgcttgaaaggctgaaaagatgggtggatgagaggtgccctgaggttccttccaactctccaagattctgtgatactcctccttgggtgctgaagacactttggcaatgtttggatttccaaacggttgcttagaaaactaagggacccacgaagcccaggtaagcctcagggtctttcccaggaatcgtttatttatttgatcttagttctgacctagagcctccaccttctccatccaaatcaccacagtggtcgaaaagggcctgtgcttgggaaatccttcctttgttggcgaatcttggacccaaaccctgcatttgggcatctgacacccataggcccatggttcaaacccaggcggctctttccaagttcgaagcagggactcggttctgcgcatctggcgggtgtgcccagattgtaaccttcgcactaggcatccaagaacgtgctttggacacactcggcggtgggagcgggtgcttggtttgcatcagggatgaggggtggagtagcgaaagcgcccggagcgcgcactcactcacactccccaagccagggcgcggccgctctgggcgcgaggtcccaagccaagtctcgcgctcggccaggagccggggagtaccccaggtccccggtctggggccgccgctatcctaccctgcggcgcgcactcctgacctggccccgccccccggcggccgcgagtagcgggcggagcgcaagcagagaggcgctctgggctgtgcggcaccgcctctcctcggtgtctggggagggacggagggaccgggcgggagagagagaaagcctgaccgaccggctggcgaagagctgcatgcaaccggtgggaggccgggccggctgggtctggggctcgggctcaggctcgcaccgtttctcggcaggtccctggcggtgagcgcggacggcccggaggcggcggctctgagctggcaggcggagggctgtctcctgcgcccgcctgcccggcgcggtccgaggatgcgggggggcgatgcccggggccagggacgcgctctgtcaccaggcgctgcagctgctggccgagctctgtgcccgtggggccctggagcacgacagctgccaggatttcatttaccacctgcgggaccgtgccagaccccggctccgcgacccaggtgagtgccgccaccgccgcctggagggacctaagggccctgggctgggacttggggccgcaccggggcctgaggcagaacagcgacgcggactgggagagggcggggggcggccaccgagccaggagagtgatcaagaaccagcgcactgtggcctgatgctgtaaccaggcggggacccgggcggtggctactgcacggaggccgactccgggtcggctagggcagggttggagggaccacgcccgccacctgcgctcccatcgccaaggctcctgggggcggctccctagctccgagctacgctctccacttcccgggctatctgcactcagagcgaggggaggccgcggcggcctcggagccgtgggaagggggacgaggccgaaggaaactctgggaagttgggagttggggacggccccgggttggggccgaatcccaccgggtgctgctcatcctttctcccaagcgtctagttcctttccttggtatggggtgagaagggcgggacgcgatccggcgttgggccggggacagggtgcttctggccttgggcagacgggcctgaacccgttcccggcgggtcgcacggcatggaggtggcctgggcctgggcgctaggctcgaccagcgactaccgccggccacgatggggttcgtagactcgtcggtcctggcttgtgcacctgtagttactcctagtgaaagttgcaaagctcttaactttggaaagttcatttattttggcatttgaccaattaaggttggaggagggagttttagtggagccatttgcttttcctgaggtccctgggtctcccaggtcgtctcatccgttactggctactggaaaatgcattccttccaatgtttgcttgagatgtttctcacctggagaaatagttttctcgaatattctcccaacctaagtacttaccaaaacagcatattgtggttggattgttaaaaatgggatgagaaaatggcctttctcttgatattgctatgtataaatattggaaatgagctattgccttgcaaattatgatgtacgaattgaaaacaatttattatataaaaatccatttatccaaaagataaattaaagaattatttgatttataaagagtgtcacactgagccagacttaaatagcatatccttgatatattcaagatggtcgtgtggggtgaagtcatctgctaaaattttaaagtttatatcagattttccctaccagaggcttttttttcctattgaaaaagaaaaaaatattgttttccattgccggtggttatattaactaagtttattgaatgatcattgatatattacttcaaagtggataattgggtagagaaatactgatcccagtggtttccagggaggtttaatgtccagcctatgtgcatctcattgctggatgtgatgtgctgaataaaccacagtcccttatctcacaagctggagtcccaggaagatgtcttaagtcggggaagttcgtccttaagcagcagaagcaagacaattattttgcatgctttgcccaaagcaagtattagtattgaaacatctcacttccttgcctgtgaccagaccacgagttctccttgttatcccacataagcagttgtctttggctttacaagagaagatggatgaggcctatctctgaatcacatctttttggccagttttctgcttctactccttttaatccgggagctggatcctgtgttctcagattctcttcagcccctacaccaggcaacccctgagaaagagtcactaagtaggcttcttacaagaggaaatctgattccttggcggaggaatagctagagataggagtcgaccttgctgctctgagttcctgatagcagtgctctgctttcttaaactaggacacaagggttttgaagtagccccatgcccctgtggctctttggtttttgttggaaagtagccagctcatgaggacaggggtgggggttttcgagtgcaaggtgagaggaaatgaggccaaagattacatccaaagtgcaccctctccttcaggacacttctcccttcatttccaaatctcccttcatttccctccttccttgccttgccgacattaattcttacatcagatgcatttgtcttcatccctctgaagcaagagtgtctttaatctcggaaccctaaaagctgagtagtagaaaagctgcaccacacaacgccgtgtgaataatgtccctgctgttgatagtgtgaaccagaacaaccatgcacagcagccctgagacatgggttcctagagagagtcatctcggtaaatggatgacttctttttctggaaaggaatccacctgaatgccatgtcccagctttctctgttattgtccattcacggccaccctgtgatagctaggttttctcaagctgaactaaggactttggttataaaaggagaaaaaagatataatttcctgtggacagcaaagtgaccctgcatctaggaaatttactcattttttcagaggtgtggaaagactagggcacagaccagcgggcctgagccatcagaaaggttcttttttgtccaggtagttatagtgccctgttgggggagtaccgcacccagatttccctgacttggtgttcacagaggacttgatgcaattg

SEQ ID NO: 65 - DNA, 5787 bp, Homo sapiens
aaaagctaacttttctggccaagcagaagaataaaagtaatcaccatgtaatattgttgtgaagactacctggccccacacattaactcagctaatcattacagtattacacggtgattatctgttccattttacagatacagaaatggaggcaaacgattaaaatacacacctaaggtcacatgcatcttgaacccaagcaatctcattttaaaatccgtgttcattgtcacaacactctattatgccctctgatctttgcattgccaatttaaatccaactttctccaactcaactttaagtacaggggtcatacccccgtcctcgccccccgcaacttgaaactccctgggaaagataacctcctggaccctggaaatgaagaggcgtatttaaaaacaaatgcctcatcctgggcacaattccaacattttatcaaaatcatgaaagtataagaatagaagtaaacaaaaagctttcatcgatcccctctcaaccataccctgagcataaaacaggtcctgtactttacagaactgtattccaaattttgctgctcttaaaaattttttttatatcattgcttcccaaatgaattacaataacgcagacatagagactgctggagatcgtgaccttaaacagttaaagatgtttgcatatactgcagcatgtttggtgtggagggtatctagagagtcctaaaaaagcaaagaggaagaaggtttgctgtacgcgtctggtgcggtggcaccgtttgccatgcccacctgctctatctcccgagtacccgggatctctccgttacagctggtttgcattgggattagcagctctttgcatgaggttgtagctgtggatgtggtttctgtagtgatggggccgactccggagatctattggctgctggctttgtaaatttcattcagtttggtccaatggcagagggagagccccggagacagcaggacctctctcctcaatctctctttttcttgcagaaccgtctctctcccttctctgtctcttagcacagagctcttattcagccactagcttggcccttcctgcttcaattgtaatgcttgttctgcccgtccacagactattggcggcagaaacaacgaatttcctccaaactaggcggtgttggtggctcttgcattcctctggatgaggaaatctagttggggggttccagaaggggaaggctcctgggctttcaatacatcctcctgaatcatacctcgtttcgggttccctagaaaaatctggacgtgtaaaaagaactcttaacggccgatgcagctcttccaaagctaaggtaggtgcagttttaagacctgtctctgggacattattctcattttaaaaagccgtttaaacattttgacttgcagcaaaggatggaaagcctcactgcagatacttgagcttcacttcatctgatctttattttttccttttatgattattaatattatttttggaaaatttggacaggactttctcccatctgtctcgctgcatttcttaggtgtgggtgggagtgtagaccttcatacggtttttacatgcaacctctccacagaaatatttggttttattttcacttaaagagaaaaatccagaccaccgttgtttggaagcgttttgctgcaatcagctatttgaacggctctggggccgtgtgtgatgtgtttacaaagtagcgctgccttccacacaaataaacagaagactgtggcggggagaggaggaaaaaaatatatatgtatctgcagtacagggagaagaaggagagaagcggccagggctggagatggtgaaggcaggaagacttctgcaaactgtgaggcatgggaggcttttcttttctttttctctccccccccaccccccccccttattctttaagaaaactgtcagctaccaccgcctggggtgcttttttgaggggttgggggggtgctgttaaccagaaagaaaaagggaaaaccggcttggttggggtcgcatttaagcgattttttttccctccttcatctccgggcctcggataagatgacggcttgggtgatgcacgaaataacgcacgtgattgattagacctggcttggcttggctagggaacgatccaggcgcgctggagaccccgcgtgaagatgaaatgacggtagctccgggctgcttctgtaaaccggggagcgggctccatgcacccctttcccgtgtgtgtgggtttcgaggcgggtgggaagggtgaggcaagccgcagaaggagggtagagctggtggttttgcttctttcggagcctttgagtgtagtctgaacctttgaggggggcgcgggggggcttgcagctgccgccctgggaaccatctctgaactgcccgcttttccgaaggagcggaaaagttggaagctgcgaggacagactaccggagccctggtctgggtctcgggggatctggagccctagtcggtgcccactgagaacaccccttctcggagcgagggtgtcggggggagtgttaagcctgcggggcgcacggtccgccagtccccgaggtggggacgggggaggaggctgaggagtcggttccaataggcgcaccacctctacagccctggaaaacgcaaccgccaccccctcttcccttccatcccatcccaagcctctctgctgtcccgggccgatttcatctcgtctcttcccccgcctccccgcttccccgcctcccaattcccgcgcggctcggctcagccccttcccactccagtgggcagaactgatggagaagatccgccaagcgcgcagccggcggcggaggagacagtgcggggtgggcgaggggcttcgagaccacgcagagagagagtgaacttcagtcctgacccctccccaaggccgcggctggggcgcccacagcccgcgctggcacccgcgtggcctgacctgcggaagcgcgagcggggatgaggtagggagagggaggtaggtgccgctcggctgcagatgatgcgtgggtggggggcttgctgtgggaggagaggcccaggtcccggcctgcgccctccactccgcggctgctccctccgcctctggttttccaagaggccggtcgctaccccggaggacactctcatccttcagtcagtctcctggacaccccttcctcctcctgtccctcaacctgacctggctctttcgcccctccgagaaccggtaggctggggtccctcggcggggttctcctgggccgcacccgaagctttgcgcccccggtatccgggcccagtgctccgtgcaaccctgggcccgagcgcacgattccggcgcctgctcgccgccagacacagcgccctttcttcccggagcggcgggggcgggagcaggggggtcaggccaacccttgcacccccgaggcctggcccgggccaccctgggaacggatgttctgcatggagagcgaggggcagccggaggacgtcctccgcatcatacccctccccttccccagaaggctttttttttttccggactgcgggtttctttttctctgccttcttcctctgaacctacggcaggtgtcagcctctttttgtgtatgtgctgctgctatctcggggatggcgggggagggggtgcaggaggcagcgtgaaggggtcctaggaggttccggcggggttttggcccctgcggtgcgccggggcttgcaactcgcccgggtgctgggcgcgcgcgtcacgaattcagcctagggcttgggcgagtctgcggggagtgaggacagaggatcccgatctgtcatttggacccaacttaagaaatttggggtgggggttgggtgggggttttggaactaagcaggtgatgttcttgcgagctggatccacaaggtggtagtatggcttctttttatttttattttattttatttctatttggtcattttttttggggggggcggtggtttgttgttgttgttgttgctcttatcttatgctttttgaaggcatccgttgcccgtagggtttacatcggagcgcgttgcattatattttcttgaaagggggtggtgtgcgtgagctcccatctcagaatcagcccttccggtgatgtgaggaaggcaaaagcaaaaaaaaaaaaaaaaaaaaaaaaaaaaaaaagaaaaaaagaaagaaaaaaaaggaaaagaaaaagtttagggagacctcgttatcctgacgaagcagaattgccagtttgtgtgggcgttctgcgggcaacatagaagtgcatgcttaagaaatccggggtagcttccttctccagctagaaattaaatggccagggtgcaaacacctgactttgatgagaacaaagcggcagaaactgcaagagacctgcatggtttgaatggacgcactgagccttttcctaggggatggcagagcggggtgaaatcagatagcaaagaaatctgccgttttgtgggggcagatttggagagtggagaattatttcatacctttagttggctgtggggaagatgttagcagtaatccattaaatcctcagcatagattttcctgtggaaatgagcaaaatgttaagtgggggagggatggctaatggcacatggttgcattaatccctgtatttccagaaaaaaatatggaatttctgtgtatcctaaaattaagaatacaggaatttcatggagaactctgcaagcatgtattttctcagattagaaattcagtattttattactcaatgaaatgtagaatgcgtgtgtgtgtatgtgtgtatacagacatacacacacgcattctacatttctacatatatgtgtgtgtgtgtgtatatatatatatatatatatatatatatatatatggccattttaaagagtattttctttgacatgtaagaacataatcagggccagttgtagcaagtggaaaattacttcatcagttttaagtcagtagattaaaatggaaggcttcatttttttttgaaatcagaataataattgcattttcataataatgcctgtgcgtggatgcagttttaaagatgctttgatgttttcttctccagtggaagaattgctacttttctttgcgttttatttaaataaactaatgccgagtatacagttggccctcaaaccagtaacctagctgatttttacccaaacctgagaatgtaacagatacttgataagggactggtggctgcataaggtagataatgaagttatcttgatgctgtgaaatttacaagcagacttgaaagaatttgaaagttcatagttgttggcctggaatgtagcctaatggtaaatatatagattttttaaaatttgtgaacttggctatttcattgttttgtgtgtagtaatttgtggaaagcttatagtctctccacaaagatgagagtgttgactgactccgcaacagagacttgcttttggaagtgcaggggtctctttaaaagccatttggaatactgtgcttttatttctagaccacaaccaaaaggttctcaaaaaactaaacattcaagtgcacgagggaatgacctccgtttaacattctttctttttaattggtacgccacatttcaaaccttttgtaatactgttgaatattgccaataatgcaacttgttgagcgaatgcattgcattcaaatgaagtagcaatatacaaatattttaagtcctttagtatcctccttctaaagataggcttatctggttaaaatatacttatattccaaataaggtga

SEQ ID NO: 66 - DNA, 5357 bp, Homo sapiens
agatgtcattcttaatattttatattctacctgtctgttcaaagaacctgaatctgtttcacgataaattttaggcctattttccagggcatttgtagatttatgtgtattatgttattatgtgattatacacaaaaagtttaagaatatgtaagaatactagttcagtagtattattgagagtaactgttgggaatacttctgcatccactatgtagttgaacacagcaaagaagaaaatcagccaaaatttaactgcaaaagaacatcacatgataaagaaagcctggttaagtggaaataacattcagattagaactaataggttcaaccaaagttttcataccagtgaagttttgttttacttgtatctgttctccataaatggagcccattagagcttaaatgcttaagttaacaaaatattaataaaatcaattgtgtaacatttctaatgaataaaattgatttaagtgtggtacaagtcattttaccaacttgcctttttagaaaaaagactggtatcattttatcaataagtaaaaaattaataaaggaaaccattcgattaaaatgtcttttgagtttctcagataaaatgtcatggctttctcaaaaacacatgtttacaaattctaaatcttggttttagtgtaattttcacaaaatgtttttaagtagcataagtaaatatccttagtaataactaagcttaagtgcattcatgttaacaaaattatttcaggaaaaataaatgttgaaactgcttacctggcaaggttgtttcattttaatggctataacatggtatctataacagcaaagttcacaggtccaggaacctctctcactgatccattttagcaggcacagctgatgtgtataccgaactgacccatcacatcggcaggggttcaacaactcaccctaaaaagaaaacagctcagtgtgaatatctgtgtctgtgtctctattttaaaaacctaatgcttcctttcttttctttcaaagccatttactcaggatacagcatcaggctctatcataggaaatgaaattaaaattttatacctctgatgggaatgaaactaagtcttaaaactatttcaaagacattgcttagagttatgacataaatgttctaccaacatacaaaatgtctgcaagatatagctcccaatacccgtgggggaaagctgtactattaaatctctttaataacttttaagtggatggtcagatgcatttcctctgcagttattcttaatcattgctaaagaagaaacgcatggcatcccagaaagcggcattatgcttttttttctaaatgtagcatcataaatattgtcttagggcactatattcctgaaagcctgtatattcttatgcaatttccttacttttaaaaacattacacctaatcttttctcagaccacacatttgtaagggacatatgctatcatttcctctgcatgattactctcatataagtttgcatgcacctacaatggtgtttataattttagaattgtgttgctgtcaaaatataaattgttggcataatagcctttctttcaaagtccattcctatttctcttgtaaaaactatcacatgcttttcacatccgtgagattaagtcacccaaatttgcagtaaggaaagactctgccctttctggttgtgatatgacatctgtctcaagatacatctgtagaaaacggacttcaccttacatctgacaggtttgcttctaagcataaaccaaaatccatcgaataattcacaaaagccgcttttggcattatcactaaagcacctgaggaacatgtagctcctaagccaggaaggaaaaaggaagtgtagatgaaattaaacccagtattcaccactgggacatttctatttgccttcacaggaagggaaaggggaaggcatattggagccagcgctcacagggcagaaccagacgagcctcactggaggcaaactgggaggtaggcgtgcgctgtccgtggtgctgaaagcttgaccggcgcgagctggagccgccaccggctgcctcggggtctcgccgggccttacctgctccgcgccctggaagcagatcttgcagatgggctggtggtgctggtgctggtgcccagcgcgctggtcgccgccgccactgctgctgctgcggctgctgcacaccgagcgcgtctcgggctggtctccggcgccccgccgctcgcgctcgccgcccgcgccggcctcagactccccggggccgcctttcgctgctgccgcctccgggaggcgcctcggaccttccccggagtcgccggccgccgccacttcctggccggcgggctgcaggggcaggggcggaggcggcagctcgtccgctcccctgcaccgcggggccacctcccctagcggctcgcttggccccgcggcgcgctcgggggtctcgggggacgcgggcagcggcggcaggtagcgcggggccgcggggaccggggccggctctcccggcggcggcgtcggcggcggcggcgggggaggttgcgggggaggctcggcgtccccgctctccgccccgcgacaccgactgccgccgtggccgccctcaaagctcatggttgtgccgccgccgccctcctgccggcccggctggcgggccgggctctggctgcagggaaagagagcgcggagggggcgggagggagaggggaaaaggagggagggggcccggacgcctggggctagggggcgggacggggaggggatgcggaaggttctgcagctgcggcggcggcaggcgcggccgttcggtggagccgccggctcggctctgatggaggcggcgccgaattcggctgcgcgtgagagccgcgccgcggaagggggggccggagaagcgagggggcgggagggaggagcggcgcggcgggggtgacggggcgcgggcgcggggtgggctgggggcgcggatcagtgggacggagttcggggttcggctccgagcgggcgggctggaagtgggggatccctcagccgcctccacgggccggccccgcgctcacgtcggttccggggcggatgacccctctccaaacggcgcagcgctgcggctctcgtgagctgggaagtagggggcaggggagaggccgcgggtccagaaaccgttactggatgggccggtgggatgtggcgcgggccgggtggggcgcgacagtctgagccgagacccgcgtgggcttaagggtgcgcgaggcgggtgccctgggcgcgcccgaactggctgagcagtggagcgggaaagggcgcgggacccgggactgtaaccgccacttccaggccctcgctccccgcgcttggagccctcaagggcactctcagggatcctcgagagccttaaaacagaagtctctggaacctgtgtcctctccctgtctgtcccgccctcgaatccctgtgtcctcctcacccgctccctcctgcagtgagcatcccgggttgttggtaaagatcttggtgcctgggaggtcggagcttcgtctcctgaaatggtttatactagtgaaccctggcgccacgttctgtggcttataatcactttcgtcgttgccgcatgaggaagcaaatgacaccgccccttaccctggaaaagtggctgcagccttccccggatcttagttttactcaccccgaagtcaatttctcggtaactccaccctgcaaaacctctgtgggactcatcttcagggcagagctaacagttttctttctggaaaaaaaaaaaaatccctcacctgcagggaactaggctgagaatcgtgcacatgcagtagtttccaaatccgtgcagtgtgagatcataaagcaccggatttatatgcggcagtgtgtctatccgaattttcactgatgtgacgctttcagtctttgacacaaagacacagagcagtcatttttggcagcgtttcatagccgaagacccaggatcagaaaggcaaaataacttgttcaaggttatagaactattaacaaagaacttctgtgttccctgaccagattttctggttcaaatgccaataattttttcttccaatcctttcttctgcctcctaaaatagtattgtagtacattgtgtgtttggcgccattgattcccactaatagatactctgtaatggctgagataaactactagcttaagttcgtttttccatttcacgttttggcagtaagaagtatatttgattcataaagaagacctacctcttttgacagaaattggttccatttgccagagagagaaggtagtgaaaaataattcaagaatgttttgtcacaaatggtctctgatgaactagtttccaggaatgtcctctaacacttagccccttcagactggctatttttggtggcagagtggcaggcagcaagcacatttttaaaacaacaatctccaggaacttaccacgttgaagaaatgtggttgtcgatatgctttcatttttggtcttcacctgtcagaatgacaacgcaagtttgtgtattgccacagtcaagataatcataagccttatgatcatcataaaccttcttcataagttctcgagttaaagactgtctagatttgtacagaatcccctaaataattccatttatgactagtaaatttctaccaggtgtgataaagtgcatcggtactcgaaggacagcataaaagctatgggttttgtataaacggtgtagaataattcagacagcgaagcggcatggataaaggagaaaagggccacaaggctagatgtgtcctctcagttaattgtttctgaaaaataaaaaaaaaaatacatttgcttgctaaattgggtagccacgtaagattccttcccggtttcaatgacctccaaagctgaccagtccctgaagggaaacttgctcgtgatcttcagttaacccaatgcattttcagtgggtccaggaaaaacaccttctttctaactttggctacatcctctgtcataaaattataaaataatggaatgcagaaaggatgaattcctctcagctgctggcaaccaagtggaagagagatggagacacagacatcagatagagcagtgcgccggtggccactgacagccatcagtagcaaggcgcatctgccaactgaaaatgaatgaggggaaatgcaggtttatcaaatttgagcaaatattgaaaggatgatcaaatcttcatgatgccagtaaaccagttgttcattcatttagttgtttaagccaaaatcctaagtgtcatccttgacttcatatcatacattcagtatacaaaaatttctttcaaatggactttcaaaat

SEQ ID NO: 67 - DNA, 5281 bp, Homo sapiens
catgatcttgttctttttatggtgaaaaagggatatttaaatatccaactataattattaatttacctatttcaccttacagttctatcagtttttgcttcaaatatttgaaagctctgttattgggaacacacgcggttaggactattatgtcctcttgaccaactaacacctttatcattatgaaatggctgtcctttctcctagcaatgttcttggctatgtaatagactttgtcagctatcaatatagtcactctagatttattttgcttagagttaactagaacatctttttccatccttttacctttaccttatttgtattttcatatttaaaagttcttttttggtaggcagcatatatctgggtcttccttttgacaatctttgccttttaattatggtatttagacaatatttaatgaacattaaccgatataattgggtttaaacctgccatcttgctatttgttctctatttgttttatctaattgtttccttcctctctttttatgccttcttttggattaatagagttttttttaacaatttcatttcatctctataggcttttaaactatagctctttgtgttatttaggaggttgctttagggttagaggacacatccttaactcattacagtctttcttaaagtgacattataccatttcacatatagcattagaaccttacaacagtatcctgccatttctcctctacctgcctttgtgttatatttgtgatatattttatttctatgtgttataaacctcacaacacactaacatttttgctttaaacacttctggctctcagcaggcatacctccggcaagcttctttctgttctcttgctagatatttgtctaaaataagaacaaataggatctgagatcataaggtttcacaatctttactgtatggtctgtgctctgaagctttttccccattgcttcctgattgttggttgtgaatttgatggataaatgaccctcaaatataagtgaattgttattctataaagaaagcagataaaaatccaaacagtgattgaaaaagtgaatttgaaataattcagtaatgaaaaagagacccaaacagtacactgttatcaattaagttaggtagacagctgggggaaatccttcatggaaaaggaggtgggtactctgcatttgtttattgtagaactagagggctggccaaaaattgcccctttttcccttttaggaactggtctaaaatgttctctcacttaatgaggcatttctcctttcccaaagccagatgtggtcgaggtattgtatattttctttagccagcagttttagatgtagaaaaacctctccaattttttaaaaaaatttattcttgaaatatgaacagtagtatgaattctagaatattgtaatgcattttattgtattcaaatacacaggataaaacagatactgtgacgacaggaagaagggagcaggctacataggagaaatacataggctatataggagaaattatgaacccggttaatacgcacagaggattttaatttaaaccttgcttgtacaccaacggagcgtttagctccctctccccagcaacttatgtctgcagtactccatactagggcaccctttctatttctattctaattctattaattagaattctatttctaattctatttctgcttcctaatcaagggcacaatacaattgtccacttctcgaatccctcctcctcctggtatctggttctcttggccctccacgttctctgttaaaatgagttctcaaaggcctcagcctagagttaagagaagaagctcgctgaggcggggcctctgggtgcccacttcagcctccccagtcatttaggacaatttcctaagtcgaagctagagatgcgtgatgcgtttggtgacttttcctgggttcttgcttaagcggagccacaggttgtgggggcggccaggcctctggggaagagcaggtgccgcggtgccggcgcgccagcagcccgcggccaaggccacctgctcccagggccccggcacctctgcggggaggcggcaggcagaggacgcgcgcgcagtcgcgcaggagccctcaggcgagtgggggaagcatcgcaagagacgcggacctggagcgcggtcactgcgttaccgcagcgacctgggttacctaagccggcgggactgagaggccgctgcgcgtgggcaggggctcagattgcgcccgcggtgcctgtcccggcccccaccccggaacgattgcagccctgcggggctcgccgcccacaccgcgctgaggacgctgcccaagccaggcgcccgcccccgcccgcggtctcagccgctcgcagcgcgccgcccccagacgcgcgtggctgctggcgagggctgagaccgcgctcccgccggccagttcccagcagcagcgcatcctccctgctccgcgccttcgctccggacccgcgtcgtcgcagccccgcggtgatgagccccgagccggtcccgccgccgccgccgccccagtgtcctggctgcgaccgcgcggagccgatcgcccagcgcttagaggagggcgacgaggccttccgcgcgggcgactacgagatggcagccgagctctttcgctccatgctagccgggctggcgcagccggaccgcggcctgtgcctgaggctgggggacgcgctggcccgcgccggccgcctccccgaagccctgggcgcgttccgcggcgccgcgcggctcggggcgctgcggccggaggagctggaagagctggcgggcggcctggtgcgcgccgtgggcctgcgcgaccggccgctgtccgcggagaacccgggcggcgagcccgaggcgcccggcgagggagggccggccccggagccccgcgcgccccgcgacctgctcggctgcccgcgctgccggcggctgctgcataagccggtgacgctgccctgcgggctcacagtctgcaagcgctgcgtggagccagggcccgcgcggccgcaggtgcggcgcgtgaacgtggtgctgagcggcctgctggagaagtgcttcccggccgagtgccggctgcgcaggctggcaggccaggcgcggagcctgcagcgccagcagcagccggaggccgcgctgctcaggtgcgaccaggccctggagctgggtgagtcggctgcggggcgggtccgcctcccctacacctgtccggcccagcagcggggtggggagctggccggtgggcccgaggctttgggctttcgagagctttgtctaggcttaggggtcactttgcttcctaaggatgggaaggtgaaaggtgggggcggccacaccctgcagccaggagaggccaagtgcaccccactggttttgtcttttccaggatgctggtaagtttcccgcggcccccggagcagctctgtaaggtcctgtaattgcttttcggcatcttctgcgctgttgaggagcggggaggatgacgaaatatttttgctcatcctgaaggaaaatgcacatagctgacacactacaggcgcagaaaggggtgccttgtgctggggcaagatttgctttgggtttcagctaatatagccaggaaggaaccgcctccactctgctgtaatcacgtcaaacgctccggctctagcctgctctctgtctcagaggccatcattagatgtcatggcgtatttaacttactagatagttacactgactagtgtgtgtagttttaaattgttcattcagtcatttccttatttatctgaggcctaaggccagcaacaggcactctcctgccttcacccagccaccacctgttaacactatttagctgaaaggaagaccaaacttctgtcatcgttttggctttgacatatgagtatgcgctctattggcagttgttttgggcagtgtgaggaggacggtttgctttctggtttcaccggttttataatagggcatatacgatttaaagcactagccgagaagacttgttgaatctcttgttactaaaataatctactaccatgctgtagtcagtgcacattttattttaaacttaggattaaggttttcccttttagaaggcacaaattataaagtgactagcattcaattttgtcttgcaaaaaaaaagatccaaatattgaacaagtgttcaatatctgtatctaaattgggatattctctgctggaatagagcacttaactcagcttttctaacgagtattctgtagtttttgcaaccaacatatccatactttaacaggcatattttttagttttctactaatttaggtattgatggtgttagagcagcactatccaattagaactattgtgtaagccacacataatttgggattttcttgtagctacattaaaagggtgaaaaaagcaggtgaaatttattttagcaatttttaaccctatacatccaaaatattatttcaacatgccttcaattttaaaaaataattgagattttacttttttaaactaagcctttgaaattcgttatatattttgcatttactgcactacccaattccaacatcaaattttcatcagaaatacttcatctgtatttagatgttggagaagatgtagaaaaatgaaaactgtgatatttatatgctgctaatgggactgcaaattatgcccctgaaaaatatttggtagtatctacagatgttaagcctgtccttggccaaatagtttcaaagaggtatatgcatatgtgcccccaaaagaatgtgcaattagatacaagagtgtatgtcattgttagagataccccaaaagtgcacacaacccaaatgtccagtaagagcagaaggggtgaatatatgattatgttttcatagcatactatacagaaagaaaataaatcaactacagttacaaactacaatgtggacaaatctcacaaacataatgttaagtggaagaagccagactcaagcatttttactatatgatcctatttatctgtaagcccaaacagacaaaattactgtaggatgttagaagtaaggacaatggttaccttggggggatgaaaagggaatagtgattgtgaggaagtatgagagggatctgtggggtagtggtgatgttctgtttcatgatttaggggatggatacacaagtgtgttcactttgcaatagttcattgcactatatacttatatttttagcttttctgtatatatattagacctaatttaaacgtttttaaaataggctatttcagtaaa

SEQ ID NO: 68 - DNA, 5563 bp, Homo sapiens
actttcttagaggcctcctgtcccaggcaacagcttggctctctgcactgttctgcttttcactgttaatccaacctgctcccaaacatcacccctcctccgcagagtgctgaagatgaagagagcccagcggcacaggggacccagagcttgcccttctgctgactcagtagtgactccgctgcctgtcatgactgatgtttccagaagggtcagggcctcctcaacctacctcttccttatgcacctttgtttgatgcaatagccctggggactctcacgattacgtttcttcttcatctattgtctccacttttatttatttatttatgtattttttagactctatcacccaggctggagtgcagcagcgcgatctcggctcactgcaacgtccgcctcctgggttcaagtgattctcctgcctcagcctcctgagtagctaggattacaggtatgtgcaccacgcccagttaatttttgtatttttagtagagatggggtttcaccatgttggccaggctggtcttgaactcctgacctcaggtgatccacccacctcggcccccaaatgtgctgggattacaggcatgagccaccgcgcccggccctactgtctccacttttaggagttcaaatccacaacctgactcactggaatgctcacaggagttcatgaactatctgagctacatggatagtctgcatgagttcaaggccactgcagacaccatgtctcagacaggcgtgtcatagtgtcaaagctcctgttaggcagcctgcagaacagcctaggccccgtgaattatgtcactagaacatcaacaattaattttgtccaatgacaaagtctgaaatgactgaaaagggtaggactcaggtaagcctgaaagaatctctcatgcgataataacaatttcatcatgaataataataatgaccatgtattgaacacttaaacttaagtcatgcgtctggcactatattaagcattttctttatattctgttatttaatctttacaggaatcctgaggtcagcatcatcccaatttcacagagtatgacatgacgtctcagagagggtaagcagcttgccaaacattccacagccaggaagctgtaggaccagcctgtgctcctaaggactggctcctggctgctatatatagaactaagcctggtggaggtgtcagaaatagaggttcacttctgtcatcaccgacctccctccacacctttgcaaaggaggaaactgagaggcagggatttccgcagagcaaggaacccaaattgctgcctcctgtgatttatacactgcaccccaagctgtaggggtaacccaaggacaaagctgtaacccaagcgggaacatatgccccatctggggccaccaaaatcttaccagcttcctcagctggtggatcggttaattcacggccacagccccctggagctgggggaaaggaaaaccagggcgtctccgcaaaccagcccagagagaggtctgcggaagggcccggaagcctgcaggcccctctgcacccccaaccccaccgccatcctggacctccaagatgacctggtccaacagagtcctgcatggaaaagactggaacccagggaggagcagagccccgcccaaggtcaccggccgagcctgaatagaacccggttctccaggagccctgtctttagctgtcttgtccaaataaaatttttcaggccatcagatttccgtactccctggagtgggacttcatctgggaccaaaggagggctggtgaggggagtggcaggagggaggagtgcctcggggccccgagcaggatgagcctgaggaagagacgggtccccatgttccctttcccgctcagataatggaggtgaattgaggggagcagagacctccccaccttcagggtgggaccctgagggaccaggacacctttgctaggggatgtccctcctcactcctgcacaagttcctcaaggacaccctcgggctccgaaaacggggggagggggacgacgccccagaggcccctgagcccctggttcttcccgaccctaagggcttttctccctcggttcccaggcggcgacggcgggtagcgcgaagcagcaggcgcaggggcgctgggatggggatgtctctgcaggtctaaggttccccttgggagtctaaacaaagactacggcagcgccgtcccctcccccgggaacccgacgccgcgcggccacagggggcctggaggggcgggcagggcctcgcagcgcacccagcacagtccgcgcggcggagcgggtgagaagtcggcgggggcgcggatcgaccggggtgtcccccaggctccgcgtcgcggtccccgctcgccctcccgcccgcccaccgggcaccccagccgcgcagaaggcggaagccacgcgcgagggaccgcggtccgtccgggactagccccaggcccggcaccgccccgcgggccgagcgcccacacccgccaaacccacgcgggcacgcccccgcggcgcaccgcccccagcccggcctccgcccctgcagccgcgggcacgcggaggggctcctggctgcccgcacctgcacccgcgcgtcggcggcgccgaagccccgctccccgcctgcgcgtctgtctcgtccgcatctccgcggtgagtcggcggcgccctcgcccctgagcccagggccagcttctctcgccgccgcggctgctgcgcgcgtccccgcccagcccagcccagccccgagcacgaccccagccccacgcacgaccctagccccgcgagtcccgcaccgactcgctcccgccccatttcgcctccgcgggggcggcgccccctcctccccgcggctcccgctctccttcctcgccttcccggccgcgctggggacccccagccgccgtccgcgaccccccaccgcgacgcccggaggcggcggggtctctttgttcgggcggcgggcacgggggaccacctcccacggtgtcaccgcacccaccccgcgcccttcctccgcctcctggagttcaccgggaccaggtggcggcgggtgcctttttgggggtgcgcggccatgcaattggtggatttttttaaaccgttttggaggggggagcgcggcgttgggggcgggagagcgctcctggctgtgagctgctcctgccgcttcgctccgcgctctcctgccgctccgctccgggtctcccgcgctcctctccccggctcggccgagcgcgctgccccgacgccgccacccagagccgggccgcgccgggcgccgagatgaaggtgctgggacaccggctggagctgctcacaggtaccgcccgcctgccccgcagccggccgccactttccgagttggagcggactccgggcgcggcggccggggactggggcggctcgggtctgagcaggaaggggtgcggaccccaactaagtcctagttttgtgctacctgtttgtgtgcggagcccagccccgggagaggacttgaggttgtggcgagtccctggcgctggcgtccgggctgcgggagcaccggtcagggggtggccccatggggtctctgaccagcggagctcggattaggaccctgaaagctagctcagggctcctgccctccaatcagtgtcgcttgtcccctaagaaaggacccgtgggcttctggcaggacccgcgccatggacctcttatttctgcgccctgtgacaatctgagccgtctttctctgggggagaagtttcttgctgggagtggaggcgacgccaagtggcctgggaagtgggaagccagattggaccctactgactggggaccctcagccttggggctcctctggagaagtgatcagttgccctgctggaaactcacatccagggggcagtggctggagagcaagagcgaacggtcaggaagaggaggtgggaaagggagcagggacgggggggaggattcgaggagtgacttctgtgttctccccggtgtggagagacccagacaggaggaaaggaaagcaacccggtttcctccagctctgggacttataggtgctccatccgtgtatgtcagatgagcacagattccagtaagtgtcctccgacacctgggggagggggctgatcactgccttccaggaccttaatgtccgatgagggagcagagccccggagccctgttacaaggctggaaggggcagccgtctgtgggtgcgctcaggaaacggtggaatccgagtcgggggcagcttttgaagacctcgaaaaacaatttttgttaatgaagaaggaggtggcattatgggttcaggatcaagggaatggcccatgctctggggtgtgagagaggcttgttcggggagaggtatggaggctgaatggcctggaatgtgcgtgtgtgttgcgggggagggggctagaaggtgggggtggggtacgttgggtgaggttttattaaatcggcagtagcaggccaggcgcagtggctcatgcctgtaatcccagcactttgtgaggctgaggccagaggatcgcttaaggccaggagttcaagaccagcctgggtaacatagcgagaccccatctttacaaaaaaataaataaataaatcagcaatagctcgtgacagacttgtagagaaggaaaggtggaagctgttcctgggacactggaaactgactgggtgctcagaagttccagcgctgagcccagttagccacggggctggcaccccctccaatcccttgccaactttttctttgtggagctcacttgaggcaggcccagctcacccaacttggaagggaaccgcgcaggccactgaacttgttaaccccctggagggaaggcttgcggttccatgggtgttttgtttgtttgttttgttttgagatggagtcccactctgtcgcccaggctggagtgcagtggcacagtctcggctccctgcagcgtccgtctcccaggatcaagggattctcgtgcctcagcttctcaagtagctgggattacaggcacgcgccaccatgcatgcctaatttttgtattcttagtagagatgggtttcgccatgttggccaggctggtctcgaacttctgacctcaagtgatccacctgcccctgcctcctaaagtgctgggattacaggcgtgagccaccgcacccagcccccaccatgggtttttagagctctgcatggtgaacagatctcctggttcctctcttccccaactcttcctgcccccaaagggcctggatgagaaatgctcacaggagacatgtgtggggggctcaagcaggggtctaggctttggagaccccatgcactgttctctcatctcctggatagaggagtcaggacccctcttcccccaccagatggggtcaccagattctggcgggctttctaagtccctggccagggtggatgaggcccctctcagttactgtcatcagtcacaggctccgcccccattgtacctgcaacctggccctgggagacaacccatcccctctgttgctattttgtggtcttcacagacttc

SEQ ID NO: 69 - DNA, 16 bp, Homo sapiens
gcggaagttgtcgtcg

SEQ ID NO: 70 - DNA, 19 bp, Homo sapiens
cgttcgtatcggtaacgtg

SEQ ID NO: 71 - DNA, 17 bp, Homo sapiens
gcgtttttcggcgagag

SEQ ID NO: 72 - DNA, 27 bp, Homo sapiens
aagagttagggtttaataagtttttcg

SEQ ID NO: 73 - DNA, 20 bp, Homo sapiens
cggacgcggagggaatgtag

SEQ ID NO: 74 - DNA, 23 bp, Homo sapiens
cataaaccgaaaacgaatacgcg

SEQ ID NO: 75 - DNA, 21 bp, Homo sapiens
cgcacccctactcaaacgacg

SEQ ID NO: 76 - DNA, 25 bp, Homo sapiens
cgctaaattaatacaatcgacgcgt

SEQ ID NO: 77 - DNA, 20 bp, Homo sapiens
gttgcgaagagtagagcgcg

SEQ ID NO: 78 - DNA, 21 bp, Homo sapiens
acgtttagtaaacggtcgtag

SEQ ID NO: 79 - DNA, 17 bp, Homo sapiens
gaaatcgcgactacccg

SEQ ID NO: 80 - DNA, 26 bp, Homo sapiens
cgcaacccctctaacaacccctctcg

SEQ ID NO: 81 - DNA, 18 bp, Homo sapiens
gattttcggtgggtagcg

SEQ ID NO: 82 - DNA, 23 bp, Homo sapiens
cgggtattgcgtttttggatacg

SEQ ID NO: 83 - DNA, 16 bp, Homo sapiens
cgaaaacgaacgcccg

SEQ ID NO: 84 - DNA, 16 bp, Homo sapiens
aggcggcgaagatgtg

SEQ ID NO: 85 - DNA, 18 bp, Homo sapiens
cgcgcggaggagatatcg

SEQ ID NO: 86 - DNA, 17 bp, Homo sapiens
attcggcggtgggagcg

SEQ ID NO: 87 - DNA, 17 bp, Homo sapiens
cgaaacccacacacacg

SEQ ID NO: 88 - DNA, 17 bp, Homo sapiens
cgcggattagtgggacg

SEQ ID NO: 89 - DNA, 18 bp, Homo sapiens
cggcgggattgagaggtc

SEQ ID NO: 90 - DNA, 20 bp, Homo sapiens
gcgtagaaggcggaagttac

SEQ ID NO: 91 - DNA, 21 bp, Homo sapiens
ccacaaccaaaacaaacatcg

SEQ ID NO: 92 - DNA, 17 bp, Homo sapiens
gctcgtactcaactcgc

SEQ ID NO: 93 - DNA, 25 bp, Homo sapiens
gctccaactacttctaaaactaacg

SEQ ID NO: 94 - DNA, 18 bp, Homo sapiens
ctcactacgctaaacccg

SEQ ID NO: 95 - DNA, 16 bp, Homo sapiens
cacgacgcccgcacct

SEQ ID NO: 96 - DNA, 16 bp, Homo sapiens
gttgtcgggacggagg

SEQ ID NO: 97 - DNA, 22 bp, Homo sapiens
cggagtggtggttatagcgagg

SEQ ID NO: 98 - DNA, 16 bp, Homo sapiens
gcgcgggttgattggt

SEQ ID NO: 99 - DNA, 20 bp, Homo sapiens
ccgctcctcccttactcgac

SEQ ID NO: 100 - DNA, 19 bp, Homo sapiens
ccgaataatccctaccgtc

SEQ ID NO: 101 - DNA, 17 bp, Homo sapiens
gcgcgcgcgtatttata

SEQ ID NO: 102 - DNA, 18 bp, Homo sapiens
gcggcgcggtcggtttcg

SEQ ID NO: 103 - DNA, 19 bp, Homo sapiens
cgccgctaaaaaactctcg

SEQ ID NO: 104 - DNA, 21 bp, Homo sapiens
cctatccctctccgaaaaccg

SEQ ID NO: 105 - DNA, 20 bp, Homo sapiens
cggtggattttcgagtttcg

SEQ ID NO: 106 - DNA, 19 bp, Homo sapiens
caacaccaacgcccaacat

SEQ ID NO: 107 - DNA, 17 bp, Homo sapiens
caacttccgacgacgcc

SEQ ID NO: 108 - DNA, 21 bp, Homo sapiens
ccaaaacgaccgcgccctaac

SEQ ID NO: 109 - DNA, 17 bp, Homo sapiens
ggaacgatttaggcgcg

SEQ ID NO: 110 - DNA, 18 bp, Homo sapiens
gaaaccgacgtaaacgcg

SEQ ID NO: 111 - DNA, 18 bp, Homo sapiens
accgaaacaaacaccgcg

SEQ ID NO: 112 - DNA, 22 bp, Homo sapiens
acgaatataaacgctcgacccg

SEQ ID NO: 113 - DNA, 21 bp, Homo sapiens
agtcgcgcgttttcggttcgc

SEQ ID NO: 114 - DNA, 21 bp, Homo sapiens
agtcgcgcgttttcggttcgc

SEQ ID NO: 115 - DNA, 34 bp, Homo sapiens
tgttaagtatcgcgggatttgcgtatatcgtaga

SEQ ID NO: 116 - DNA, 27 bp, Homo sapiens
agttggagatttcgatagagcgtcggt

SEQ ID NO: 117 - DNA, 20 bp, Homo sapiens
cgcgtagcgcgggcggtgcg

SEQ ID NO: 118 - DNA, 28 bp, Homo sapiens
cgcgttcgggcgttcggttatagttgcg

SEQ ID NO: 119 - DNA, 29 bp, Homo sapiens
ccactaccgacctccaaacgcctctcccg

SEQ ID NO: 120 - DNA, 20 bp, Homo sapiens
tcgtgtttggcgggggtcgg

SEQ ID NO: 121 - DNA, 16 bp, Homo sapiens
tggcggcggcgcggag

SEQ ID NO: 122 - DNA, 34 bp, Homo sapiens
agtaagcgtttattttcgtcggatagggtaggtg

SEQ ID NO: 123 - DNA, 19 bp, Homo sapiens
atcgcgtacgcggggttgc

SEQ ID NO: 124 - DNA, 36 bp, Homo sapiens
cccaacgcccctaaaccgtcctaacccgaaccgacc

SEQ ID NO: 125 - DNA, 25 bp, Homo sapiens
cggttgtagggagtttcggttcgcg

SEQ ID NO: 126 - DNA, 27 bp, Homo sapiens
cgcgcggttttggatttggtggcgacg

SEQ ID NO: 127 - DNA, 39 bp, Homo sapiens
cggtttagttggaggaggttagttttataggcggtagga

SEQ ID NO: 128 - DNA, 34 bp, Homo sapiens
cgggtgtatgggagtatcgagtagtttgagttgg

SEQ ID NO: 129 - DNA, 29 bp, Homo sapiens
cgcggttgaggcgtttcgaagggtaagcg

SEQ ID NO: 130 - DNA, 29 bp, Homo sapiens
ggtggagtagcgaaagcgttcggagcgcg

SEQ ID NO: 131 - DNA, 29 bp, Homo sapiens
cgcgtgaagatgaaatgacggtagtttcg

SEQ ID NO: 132 - DNA, 21 bp, Homo sapiens
cgagcgggcgggttggaagtg

SEQ ID NO: 133 - DNA, 26 bp, Homo sapiens
cgcgtgggtaggggtttagattgcgt

SEQ ID NO: 134 - DNA, 20 bp, Homo sapiens
agggatcgcggttcgttcgg

(19) United States
(12) Patent Application Publication
(10) Pub. No.: US 2023/0242995 A1
(43) Pub. Date: Aug. 3, 2023

(54) METHOD FOR DETECTING COLORECTAL CANCER

(71) Applicants: EPIFY B.V., Maastricht (NL); UNIVERSITEIT MAASTRICHT, Maastricht (NL); ACADEMISCH ZIEKENHUIS MAASTRICHT, Maastricht (NL)

(72) Inventors: Manon Van Engeland, Maastricht (NL); Alexander Koch, Maastricht (NL); Leander Van Neste, Maastricht (NL)

(21) Appl. No.: 18/013,178

(22) PCT Filed: Jun. 29, 2021
(86) PCT No.: PCT/IB2021/055828

§ 371 Date: Dec. 27, 2022

Related U.S. Application Data
(60) Provisional application No. 63/045,468, filed on Jun. 29, 2020.

(51) Int. Cl. C12Q 1/6886

(52) U.S. Cl. CPC C12Q 1/6886; C12Q 2600/158; C12Q 2600/154

(57) ABSTRACT

The disclosed subject matter is in the field of molecular diagnostics, and in particular provides diagnostic markers, methods, systems, and kits for detecting a predisposition to, or the presence of colorectal cancer (CRC), such as detecting a change in the expression status or methylation status, or a combination thereof of any one or more of a number of genes. Also described are pharmacogenetic methods for determining suitable treatment regimens for cancer and methods for treating cancer patients, based around selection of the patients according to the disclosed methods.

### CROSS-REFERENCE TO RELATED PATENT APPLICATIONS

The present application claims the benefit of priority under 35 U.S.C. § 119(e) to U.S. Provisional Application No. 63/045,468, filed on Jun. 29, 2020, the content of which is incorporated herein by reference in its entirety.

### TECHNICAL FIELD

The present invention is in the field of molecular diagnostics. In particular the invention provides diagnostic markers, methods, systems, and kits for detecting a predisposition to, or the presence of colorectal cancer (CRC), such as detecting a change in the expression status or methylation status, or a combination thereof of any one or more of a number of genes. Also described are pharmacogenetic methods for determining suitable treatment regimens for cancer and methods for treating cancer patients, based around selection of the patients according to the methods of the invention.

The markers disclosed herein provide a higher sensitivity and/or specificity in comparison to conventional markers.

### BACKGROUND

Colorectal cancer (CRC), also known as bowel cancer and colon cancer, is the development of cancer from the colon or rectum (parts of the large intestine). A cancer is the abnormal growth of cells that have the ability to invade or spread to other parts of the body. Signs and symptoms may include blood in the stool, a change in bowel movements, weight loss, and general fatigue.

Most colorectal cancers are due to old age and lifestyle factors with only a small number of cases due to underlying genetic disorders. Some other risk factors include diet, obesity, smoking, and lack of physical activity. Dietary factors that increase the risk include the consumption of red and processed meat as well as alcohol. Another risk factor is inflammatory bowel disease, which includes Crohn's disease and ulcerative colitis. Some of the inherited genetic disorders that can cause colorectal cancer include familial adenomatous polyposis and hereditary non-polyposis colon cancer; however, these represent less than 5% of cases. Colorectal cancer typically starts as a benign tumor, often in the form of a polyp, which over time becomes cancerous.

The current gold standard diagnostic test for colorectal cancer is colonoscopy, an invasive procedure during which a trained gastroenterologist checks the rectum and colon for polyps and other types of abnormal tissues and removes them if necessary. Tissue biopsies may also be collected that can then be inspected by a pathologist to determine whether they are pre-cancerous or cancerous. Much effort has been exerted into the development of minimally or non-invasive diagnostic tests that can precede or even replace colonoscopies, reducing the number of unnecessary invasive procedures and potentially increasing the participation rates in population-based screening programs.

One promising avenue for novel diagnostic tests is DNA methylation, an epigenetic mechanism that controls gene expression through the chemical binding of methyl, a small molecule, to specific genomic locations. Generally, in the transformation process of a normal cell to a cancer cell, hundreds of genes are gradually silenced or activated. Hypomethylation, in general, arises earlier and is linked to chromosomal instability and loss of imprinting, whereas hypermethylation is typically associated with promoters (or enhancers) and can result in (tumor suppressor) gene silencing, which also provides a target for epigenetic therapy. Typically, there is hypermethylation of tumor suppressor genes and hypomethylation of oncogenes. The presence of DNA methylation in the promoter region of a gene most often prevents the transcription of this gene. DNA methylation is a vital biological process involved in many developmental processes, while aberrant DNA methylation patterns are found in virtually every type of human cancer.

Commercially available, DNA methylation-based tests for the early detection of colorectal cancer have been described [7-10]. There is the multi-omic Cologuard® test commercialized by Exact Sciences, which includes two DNA methylation markers (NDRG4 and BMP3) and is performed on stool. Another test concerns the Epi ProColon® test commercialized by Epigenomics, which is based on the SEPT9 biomarker and is performed on blood. The new official gene symbol for this marker is SEPTIN9.

In asymptomatic persons at average risk for colorectal cancer, the Cologuard® test achieved a sensitivity of 92.3% and specificity of 89.9% for detecting colorectal cancer, compared to 73.8% and 96.4% for the fecal immunochemical test (FIT; Imperiale et al., 2014, clinical trial NCT01397747). While Cologuard® offers higher sensitivity than FIT, it also results in more false positives [1].

In a different clinical trial (NCT01580540), the blood-based Epi ProColon® test achieved a sensitivity of 73.3% and a specificity of 81.5%, compared to 68.0% and 97.4% for FIT (Johnson et al., 2014). In this case, the specificity was lower for the DNA-methylation based test than for FIT. The advantage of the Epi ProColon® test is that it is blood-based and could therefore increase screening participation. A study in Germany showed that of the people that refuse to undergo a colonoscopy, 97% accepted a non-invasive alternative (such as a stool or blood-based test) and that of these 97%, 83% preferred a blood test (Adler et al., 2014).

Therefore, there is room for improvement of both FIT and existing DNA methylation-based methods with regards to sensitivity and specificity in the field of CRC.

Noninvasive methods may also help in detecting cancer at very early stages, even before they become malignant, if one knows the correct markers for evaluation. Pre-cancer that goes unchecked may ultimately become cancerous. Methylation-based tests may be helpful in identifying increased risk of cancer, which should serve as a reminder to stay current with medical visits and screening tests and communicate concerns or changes to the doctor. Improved sensitivity of such tests and methods can for instance be a huge contributor in identifying dysplastic adenoma and other high-risk adenoma's that will likely evolve to malignant CRC.

Once CRC is diagnosed, the clinician has to take the next step in the clinical management, which may include determining the prognosis for this patient or estimating the likely progression of the cancer and the aggressiveness that it may exhibit (recurrence likelihood, progression and/or chance for metastasis despite adjuvant therapy). The clinician then may tailor the treatment and therapy to this likely evolution of the disease. The current practice for prognosis assessment is based on radiological (CT, MRI) and pathological (TNM, lymphovascular, perineural, and venous invasion) criteria. However, none of these prognostic tools provides clear evidence on which CRC cases are more prone to relapse. Prognostic biomarkers, including DNA methylation markers, have been published in the literature. However, to date, none of the markers has the robustness to be translated in clinical practice [12]. In addition, certain biomarkers can be used to detect minimal residual disease (MRD) and determine whether treatment and/or therapy is working, or if cancer has recurred, allowing the clinician to potentially adjust the patient management.

The final step in the clinical management of a patient with cancer is the determination of the most suitable therapeutic regimen. It is well known that patients with cancer exhibit different responses to certain therapies and some patients can benefit the most from the use of a biologic factor. Thus, it is of importance to be able to predict the most likely response to a given therapy. For this reason, predictive biomarkers are being evaluated. But, also here, more accurate biomarker panels are needed for use in clinical settings.

Accordingly, non-invasive methods and their biomarkers for evaluation may be helpful in early detection, diagnosis, therapy choices, patient stratification for therapy, treatment monitoring, detection of minimal residual disease, and recurrence monitoring.

One object underlying the present invention is therefore to find a marker or set of markers that improve the sensitivity and/or specificity of tests in the field of CRC. The marker and set of markers find their utility in screening, monitoring, stratification, and therapeutic set ups and find their applications in kits suitable for such utility.

### SUMMARY

The disclosed subject matter relates to methods, systems, and kits for detecting alterations in expression status of one or more genes in a sample containing or suspected of containing colorectal tumor or colorectal cancer nucleic acid taken from a human subject. The methods may comprise of, and the systems and kits may be used for, performing an assay on the sample and detecting expression status of at least one gene in the sample that differs from a reference expression status, wherein the at least one gene is selected from the genes listed in Table 1.

Equally important, the disclosed subject matter concerns methods, systems, and kits for detecting a predisposition to, or the presence of colorectal cancer in a sample containing or suspected of containing colorectal tumor or colorectal cancer nucleic acid taken from a human subject comprising detecting alterations in expression status in said sample, comprising: performing an assay on the sample and detecting the expression status of at least one gene that differs from a reference expression status, wherein the at least one gene is selected from the genes listed in Table 1, and, wherein the detection of an altered expression is indicative of a predisposition to, or the presence of colorectal cancer.

The disclosed subject matter also concerns methods, systems, and kits for determining a prognosis of colorectal cancer and/or determining a predisposition to colorectal cancer in a sample containing or suspected of containing colorectal tumor or colorectal cancer nucleic acid taken from a human subject comprising detecting alterations in expression status in said sample, comprising: performing an assay on the sample and detecting the expression status of at least one gene that differs from a reference expression status, wherein the at least one gene is selected from the genes listed in Table 1, and, wherein the detection of an altered expression is indicative for cancer development, likely evolution of the disease, or predisposition to colorectal cancer. The disclosed methods, systems, and kits may be performed in order to characterize a stage of colorectal cancer development.

The disclosed subject matter also provides methods, systems, and kits for detecting alterations in methylation status in a sample containing or suspected of containing colorectal tumor or colorectal cancer nucleic acid taken from a human subject, the method comprising: performing an assay on the sample and detecting a methylation status of at least one gene that differs from a reference methylation status, wherein the at least one gene is selected from the genes listed in Table 1.

The disclosed subject matter also relates to methods, systems, and kits for detecting a predisposition to, or the presence of colorectal cancer in a sample containing or suspected of containing colorectal tumor or colorectal cancer nucleic acid taken from a human subject comprising detecting alterations in methylation status in said sample, comprising: performing an assay on the sample and detecting the methylation status of at least one gene that differs from the reference methylation status, wherein the at least one gene is selected from the genes listed in Table 1, and, wherein the detection of an altered in methylation status is indicative of a predisposition to, or the presence of colorectal cancer.

The disclosed subject matter also concerns a method of prognosis to colorectal cancer or predisposition to cancer in a sample containing colorectal tumor or colorectal cancer nucleic acid taken from a human subject comprising detecting alterations in methylation status in said sample, comprising: performing an assay on the sample and detecting alterations in methylation status of at least one gene that differs from the reference methylation status, wherein the at least one gene is selected from the genes listed in Table 1, and, wherein the detection of alterations in methylation status is indicative for cancer development, likely evolution of the disease, whether pre- or post-treatment, or predisposition to colorectal cancer.

Further provided are kits for detecting alterations in expression status in a sample containing colorectal tumor or colorectal cancer nucleic acid, the kits comprising: tools to specifically detect the methylation status or promotor methylation status of at least one of the genes listed in Table 1.

The gene or genes for use in the disclosed methods, systems, and kits may be chosen from: TRBJ2-2, TRBJ2-4, SLC18A3, RIPPLY2, GALR1, FAM19A4, SYT9, KCNA1, TMEM196, VSX1, MMD2, GPM6A, SNAP91, PRKG2, SLC27A6, UNC5D, NOS1, TRHDE, STOX2, C12orf56, GALNT13, TMEM130, GPR27, CRTAC1, NRG3, PTPRT, NLGN4X, SLITRK5, RIMS2, COL25A1, CNNM1, EIF4E3, VIPR2, KCNIP4, SLC35F3, ZNF542, LRRC7, STK32B, TRBJ2-2P, FAM184B, PREX2, CACNA1A, GLB1L3, PCDH7, PDE6B, COL23A1, NCAM2, FBLIM1, KRBA1, KY, AMPH, ZNF132, ATP8B2, GALNTL6, KIAA1522, TRBJ2-1, TM6SF1, RNF175, NALCN, FAM162B, TRBJ2-3, SLC8A1, DOK6, TRBJ2-7, PDE8B, GUCY1A3, WDR86, LAYN, NXPH2, MARCH11, IRX4, KCNA6, RXFP3, TRBJ2-5, TRBJ2-6, FBXL21, BHLHE23, UNCX, NPY2R, SPOCK3, C8orf56, SLC6A2, SORCS3, HBM, PHF21B, DPY19L2P4, GPR123, DBX2, OPRK1, SLC5A7, DPP10, CTNND2, SHISA9, GRIN2A, CPEB1, GRIK3, SNTG2, WDR17, SEZ6L, LUZP2, TLL1, ARHGAP20, VWC2, PCDH8, BAALC, RNF150, EMILIN3, SLC8A3, PRKCB, ZNF471, VAT1L, HCG4, RBM24, ZNF415, NPPC, SLC35F1, FAM163A, ATP8A2, TUB, ZNF549, CLSTN2, FIGN, EFEMP1, TMEM108, RSPO3, PDE1C, COL14A1, VEPH1, MATK, RNF165, SLC18A2, ZNF788, ZNF880, HSPA1A, TMEM132E, FBXL7, DSCAM, OLIG1, FAM110B, ZNF660, ST8SIA5, GRID1, PNMAL1, RGS7BP, ST8SIA2, TRIM67, SNX32, DLGAP3, FAM155A, EVC, ROBO3, MGAT3, PRDM16, ITPKB, C1QL3, GPR83, ADARB2, HSPA1L, LY6H, LRRFIP1, ZNF804B, C17orf102, DMRT1, PAX7, PTF1A, NGB, ZNF804A, DPYSL3, GLRB, GUCY1B3, ZSCAN5A, BARHL2, LONRF2, RSPO2, CASR, SORCS1, GSG1L, PCDH10, CNGA3, SLC6A15, INA, MAL, RELN, GFRA1, DCLK1, ADHFE1, KCNQ5, IRF4, THBS4, CHL1, SCTR, SLC16A12, SLIT3, UNC5C, BEND5, SLIT2, SFRP2, ITGA8, GDF6, GDNF, BVES, IKZF1, ZEB2, NRG1, ZNF625, ZNF568, SYNE1, CYP1B1, SPG20, NELL1, SFMBT2, USP44, CD8A, ITGA4, EFS, EVL, PTPRM, GPC6, CRHR2, AKR1B1, GRASP, CD34, FGF12, CDH2, ADAMTS5, SDC2, FOXE1, SOX21, PRDM14, PAX1, SH3GL3, VSTM2A, NKX6-2, LMX1A, EPHA6, NKX2-2, LIFR, HAND2, EPHA5, BEND4, NTNG1, GNAO1, LRAT, JPH3, JAM2, ATP1B2, RGMA, ADD2, ADAMTS1, PPP1R16B, PDGFD, ZSCAN18, ZNF304, ITIH5, ZNF43, ZNF582, ST8SIA1, SYT6, JAM3, HCK, ELMO1, NDRG4, FOXF1, VAV3, VIM, SCGB3A1, GATA5, ZNF331, VEGFC, GDF1 and CLDN10.

### BRIEF DESCRIPTION OF THE DRAWINGS

FIG. 1; Example distribution of the AUC values of all 1540 three-marker panels, including the AUC cutoff, for the comparison of normal and CRC samples.

### DETAILED DESCRIPTION

Colorectal cancer (CRC) is among the most common cancers. In order to help clinicians optimize their practice, it is crucial to introduce more effective tools that will improve not only early diagnosis, but also prediction of the most likely progression of the disease and response to (chemo) therapy or other treatments.

The purpose of the invention is to provide accurate biomarkers for diagnostic, prognostic and predictive purposes that either alone or as part of a panel would help improve the patient's clinical management.

Through a careful and exhaustive analysis of large-scale cancer genomics datasets, we have established a list of highly sensitive and specific biomarkers with alterations in their expression statuses, applicable for the early detection of colorectal cancer. The biomarkers are listed in Table 1.

Validation studies have been performed and consisted of testing a subset of 10 biomarkers from the list in three solid tissue sample types: i) normal tissue, ii) adenoma tissue (adenomas are non-malignant lesions that can develop into colorectal cancer), and ii) colorectal cancer tumor tissue. The same biomarkers have also been tested in stool samples from people with the same three conditions (healthy, adenoma, colorectal cancer).

Additional validation studies using 22 of the markers in Table 1 have been performed on stool samples from patients with the same three conditions. Nineteen (19) markers were tested on blood.

The disclosed subject matter thus generally relates to novel biomarkers, biomarker panels, methods exploiting the markers and panels, and systems and kits for performing said methods for analyzing colorectal tumor or colorectal cancer samples taken from a human subject.

Hence, the disclosed subject matter relates to methods, systems, and kits for detecting alterations in expression status in a sample containing or suspected of containing colorectal tumor or colorectal cancer nucleic acid taken from a human subject. The methods may comprise and the systems and kits may be utilized for performing an assay on the sample and detecting the expression status of at least one gene that differs from a reference expression status; wherein the at least one gene is selected from the genes listed in Table 1.

In a related manner, the disclosed subject matter also relates to methods, systems, and kits for determining a predisposition to, or the presence of colorectal cancer in a sample containing or suspected of containing colorectal tumor or colorectal cancer nucleic acid taken from a human subject comprising detecting alterations in expression status in said sample, comprising: performing an assay on the sample and detecting the expression status of at least one gene that differs from a reference expression status, wherein the at least one gene is selected from the genes listed in Table 1, and, wherein the detection of an altered expression is indicative of a predisposition to, or the presence of colorectal cancer.

Most often the reference expression status will be a predetermined reference expression status. The reference expression status, predetermined expression status, or normal expression value is usually referred to as “cut-off value”, “reference value”, “reference level”, or “predetermined reference value”.

Accordingly, the disclosed subject matter relates to methods, systems, and kits for determining whether a subject has a predisposition to, or the presence of colorectal cancer (CRC) comprising the steps of: determining the expression status from one or more genes listed in Table 1 in a sample from the subject and comparing said expression status of each of said genes with a predetermined reference status of each of said genes, wherein it is concluded that the subject has a predisposition to, or the presence of CRC if at least one of said genes have an altered expression status relative to the predetermined reference status of at least one of said genes. In another set up, it is concluded that the subject has a predisposition to, or the presence of CRC if at least two, three, or more of said genes has an altered expression status relative to the predetermined reference status of at least two, three, or more of said genes.

A skilled person may be well aware of ways of obtaining such a reference expression status or predetermined reference expression status. It may for instance be the value obtained using the same probes and methods as described herein when applied to a normal individual or a panel of normal individuals. It may also be an arbitrary chosen value or it may be determined by trial and error.

In some embodiments, the term “reference expression status” or “predetermined reference expression status” as used herein means the expression status of a particular gene when measured as described above in a sample from an individual not suffering from CRC. Suitable normal subjects may include a subject that is negative for any colon abnormality in colonoscopy. Suitable subjects may include human subjects.

The reference value or predetermined reference status may also be determined empirically by the skilled person. The skilled person is well aware of the ins and outs of determining a reference status for measuring and determining the expression status of a gene.

The disclosed subject matter also concerns methods, systems, and kits for determining a prognosis of colorectal cancer and/or determining a predisposition to colorectal cancer in a sample containing or suspected of containing colorectal tumor or colorectal cancer nucleic acid taken from a human subject comprising detecting alterations in methylation status in said sample, comprising: performing an assay on the sample and detecting alterations in methylation status of at least one gene that differs from a reference methylation status, wherein the at least one gene is selected from the genes listed in Table 1, and, wherein the detection of an alteration in methylation status is indicative for cancer development or predisposition to colorectal cancer.

Such method of prognosis of colorectal cancer can be applied pre- or post-treatment. Testing can be performed diagnostically or in conjunction with a therapeutic regimen. Testing can be used to determine what therapeutic or preventive regimen to employ on a patient and be used to monitor efficacy of a therapeutic regimen or treatment/surgery.

Accordingly, the disclosed subject matter also provides a method for predicting the likelihood of successful treatment of a cancer as defined herein, such as CRC, comprising measurement of alterations in expression status and/or methylation status of at least one gene that differ from the reference expression and/or methylation status, wherein the at least one gene is selected from the genes listed in Table 1, wherein an altered expression status and/or methylation status indicates the efficacy of treatment/likelihood of resistance to treatment.

Alternatively, the method for predicting the likelihood of successful treatment of a cancer as defined herein, such as CRC, comprises measurement of alterations in expression status and/or methylation status of at least one gene that differ from the reference expression and/or methylation status, wherein the at least one gene is selected from the genes listed in Table 1, wherein an un-altered expression status and/or methylation status indicates the efficacy of treatment/likelihood of resistance to treatment.

TABLE 1
List of highly sensitive and specific biomarkers with alterations in
their expression statuses, applicable for tests in the field of CRC.

| | | | | | | | |
|---|---|---|---|---|---|---|---|
| TRBJ2-2 | TRBJ2-4 | SLC18A3 | RIPPLY2 | GALR1 | FAM19A4 | SYT9 | KCNA1 |
| TMEM196 | VSX1 | MMD2 | GPM6A | SNAP91 | PRKG2 | SLC27A6 | UNC5D |
| NOS1 | TRHDE | STOX2 | C12orf56 | GALNT13 | TMEM130 | GPR27 | CRTAC1 |
| NRG3 | PTPRT | NLGN4X | SLITRK5 | RIMS2 | COL25A1 | CNNM1 | EIF4E3 |
| VIPR2 | KCNIP4 | SLC35F3 | ZNF542 | LRRC7 | STK32B | TRBJ2-2P | FAM184B |
| PREX2 | CACNA1A | GLB1L3 | PCDH7 | PDE6B | COL23A1 | NCAM2 | FBLIM1 |
| KRBA1 | KY | AMPH | ZNF132 | ATP8B2 | GALNTL6 | KIAA1522 | TRBJ2-1 |
| TM6SF1 | RNF175 | NALCN | FAM162B | TRBJ2-3 | SLC8A1 | DOK6 | TRBJ2-7 |
| PDE8B | GUCY1A3 | WDR86 | LAYN | NXPH2 | MARCH11 | IRX4 | KCNA6 |
| RXFP3 | TRBJ2-5 | TRBJ2-6 | FBXL21 | BHLHE23 | UNCX | NPY2R | SPOCK3 |
| C8orf56 | SLC6A2 | SORCS3 | HBM | PHF21B | DPY19L2P4 | GPR123 | DBX2 |
| OPRK1 | SLC5A7 | DPP10 | CTNND2 | SHISA9 | GRIN2A | CPEB1 | GRIK3 |
| SNTG2 | WDR17 | SEZ6L | LUZP2 | TLL1 | ARHGAP20 | VWC2 | PCDH8 |
| BAALC | RNF150 | EMILIN3 | SLC8A3 | PRKCB | ZNF471 | VAT1L | HCG4 |
| RBM24 | ZNF415 | NPPC | SLC35F1 | FAM163A | ATP8A2 | TUB | ZNF549 |
| CLSTN2 | FIGN | EFEMP1 | TMEM108 | RSPO3 | PDE1C | COL14A1 | VEPH1 |
| MATK | RNF165 | SLC18A2 | ZNF788 | ZNF880 | HSPA1A | TMEM132E | FBXL7 |
| DSCAM | OLIG1 | FAM110B | ZNF660 | ST8SIA5 | GRID1 | PNMAL1 | RGS7BP |
| ST8SIA2 | TRIM67 | SNX32 | DLGAP3 | FAM155A | EVC | ROBO3 | MGAT3 |
| PRDM16 | ITPKB | C1QL3 | GPR83 | ADARB2 | HSPA1L | LY6H | LRRFIP1 |
| ZNF804B | C17orf102 | DMRT1 | PAX7 | PTF1A | NGB | ZNF804A | DPYSL3 |
| GLRB | GUCY1B3 | ZSCAN5A | BARHL2 | LONRF2 | RSPO2 | CASR | SORCS1 |
| GSG1L | PCDH10 | CNGA3 | SLC6A15 | INA | MAL | RELN | GFRA1 |
| DCLK1 | ADHFE1 | KCNQ5 | IRF4 | THBS4 | CHL1 | SCTR | SLC16A12 |
| SLIT3 | UNC5C | BEND5 | SLIT2 | SFRP2 | ITGA8 | GDF6 | GDNF |
| BVES | IKZF1 | ZEB2 | NRG1 | ZNF625 | ZNF568 | SYNE1 | CYP1B1 |
| SPG20 | NELL1 | SFMBT2 | USP44 | CD8A | ITGA4 | EFS | EVL |
| PTPRM | GPC6 | CRHR2 | AKR1B1 | GRASP | CD34 | FGF12 | CDH2 |
| ADAMTS5 | SDC2 | FOXE1 | SOX21 | PRDM14 | PAX1 | SH3GL3 | VSTM2A |
| NKX6-2 | LMX1A | EPHA6 | NKX2-2 | LIFR | HAND2 | EPHA5 | BEND4 |
| NTNG1 | GNAO1 | LRAT | JPH3 | JAM2 | ATP1B2 | RGMA | ADD2 |
| ADAMTS1 | PPP1R16B | PDGFD | ZSCAN18 | ZNF304 | ITIH5 | ZNF43 | ZNF582 |
| ST8SIA1 | SYT6 | JAM3 | HCK | ELMO1 | NDRG4 | FOXF1 | VAV3 |
| VIM | SCGB3A1 | GATA5 | ZNF331 | VEGFC | GDF1 | CLDN10 | |

Nomenclature of the genes in Table 1 is according to the HUGO Gene Nomenclature Committee, which is responsible for approving unique and unambiguous symbols and names for human genes.

In alternative embodiments, a combination of markers from the established list of highly sensitive and specific biomarkers may be utilized. The disclosed methods may also be performed using the expression status of at least two, three, four, five, six, seven, eighth, nine, ten genes, or more genes from the genes listed in Table 1. In such case, a panel of genes is used to obtain a better specificity and/or sensitivity of the method. The determination of the expression status of a panel of genes may be part of a multiplex set up in which all genes of the panel are simultaneously assessed for their expression. Whereas multiplexing undoubtedly has certain advantages, looking at one gene at a time may be necessary in order to solve certain technical challenges. Thus, alternatively, one or more of the genes are separately assessed for their expression status. In such case, a combination of the separate read-out results is used as basis for a colorectal cancer indication.

We analyzed an initial subset of genes from Table 1, those genes are ADHFE1, AMPH, FAM184B, FAM19A4, FBLIM1, GALNT13, GDNF, GFRA1, HAND2, ITGA4, KCNIP4, LONRF2, LRRC7, MARCHF11, NALCN NDRG4, NLGN4X, PCDH7, SLC27A6, SLC35F3, SLC6A2, SNAP91, SORCS1, SYT9, TM6SF1, TMEM130, TRI-IDE, UNC5C, and VIPR2. Thus, in one embodiment, the at least one, two, three, four, five, six, seven, eighth, nine, ten, or more gene(s) is (are) preferably chosen from these 29 genes. A reduced subset of genes from Table 1, are genes TM6SF1, FAM19A4, FBLIM1, SLC35F3, SLC6A2, TMEM130, PCDH7, VIPR2, AMPH, TRHDE, GALNT13, NALCN KCNIP4, NLGN4X, LRRC7, FAM184B, SYT9, MARCHF11.

Implementation of smaller panels of genes in diagnostic methods is interesting from a cost-effectiveness and assay complexity perspective. We observed that the performance of a minimal panel of 3 genes consisting of ADHFE1, ITGA4, and LONRF2 in the disclosed methods has certain applicability. The specificity of such a method remains 100%, whereas the sensitivity decreases to 72% when requiring two genes and to 44% when requiring three genes to be simultaneously positive for an overall positive test result. Such methods may be useful when the specificity of the assay is particularly important. This may for instance be the case in a population that has been prescreened for CRC with a method that has a high sensitivity and a low specificity. Such a population comprises a number of false positives that may then be submitted to an assay according to the disclosed methods. This will then detect the false positives and identify them correctly as CRC negatives. It may also be possible to combine FIT with methylation markers and balance its non-specificity in an algorithm.

Thus, in one embodiment, the panel comprises any one, two or three of the genes ADHFE1, AMPH, FAM184B, FAM19A4, FBLIM1, GALNT13, GDNF, GFRA1, HAND2, ITGA4, KCNIP4, LONRF2, LRRC7, MARCHF11, NALCN, NDRG4, NLGN4X, PCDH7, SLC27A6, SLC35F3, SLC6A2, SNAP91, SORCS1, SYT9, TM6SF1, TMEM130, TRHDE, UNC5C, and VIPR2. In order to improve the sensitivity of the disclosed methods, the methods may comprise determining the expression status of a panel of genes comprising the at least two genes, up to three genes, up to four genes, up to five genes, . . . up to ten genes.

An altered expression status will lead to an indication of colorectal cancer. As such, the invention relates to methods, systems, and kits for detecting a colorectal cancer as described herein wherein the indication of colorectal cancer is based on the detection of an altered expression status of said gene or said genes from Table 1. More particularly, detection of an altered expression status of at least one or more of the genes ADHFE1, AMPH, FAM184B, FAM19A4, FBLIM1, GALNT13, GDNF, GFRA1, HAND2, ITGA4, KCNIP4, LONRF2, LRRC7, MARCHF11, NALCN NDRG4, NLGN4X, PCDH7, SLC27A6, SLC35F3, SLC6A2, SNAP91, SORCS1, SYT9, TM6SF1, TMEM4130, TRHDE, UNC5C, and VIPR2, may lead to such indication. In some embodiments, colorectal cancer is indicated if at least two of said genes have an altered expression status relative to the respective reference expression status of said at least two genes. Suitable combinations of genes may include ADHFE1 and ITGA4, or, ADHFE1 and LONRF2, or, ITGA4 and LONRF2.

In some embodiments, colorectal cancer is indicated if at least three of said genes have an altered expression status relative to the respective reference expression status of said at least three genes. The disclosed subject matter also relates to a method as described herein wherein it is concluded that the subject has CRC if all three of genes ITGA4, ADHFE1, and LONRF2 have an altered expression, for instance a lower expression status relative to the respective reference expression status of said three genes.

The expression status of the genes, mentioned above, may be determined in a number of ways, all well known to the skilled person. It may be done by determining the RNA level expressed by these genes or even the level of expression of the proteins encoded by these genes. For gene expression analysis there are a number of routine techniques available such PCR-based techniques, mRNA northern blotting, nuclease protection assays, in situ hybridization, microarrays and many more. Protein expression may be detected by western blotting or immunohistochemistry. Within the category of PCR-based approaches, both end-point PCR and real-time PCR have been used, and the most common used PCR-based approach is quantitative RT-PCR.

In one embodiment, the expression status of the one or more genes under investigation is determined by measuring the DNA methylation status of the gene or DNA methylation status of their promoters relative to a normal status, cut-off level or any other suitable reference level or predetermined reference level. Whenever the term “methylation” is used herein, it refers to DNA methylation. In one embodiment, at least one gene of which the DNA methylation status or the DNA methylation status of its promoter is determined is chosen from the genes listed in Table 1. In one embodiment at least one gene of which the DNA methylation status or the DNA methylation status of its promoter is determined is chosen from the subset of genes ADHFE1, AMPH, FAM184B, FAM19A4, FBLIM1, GALNT13, GDNF, GFRA1, HAND2, ITGA4, KCNIP4, LONRF2, LRRC7, MARCHF11, NALCN NDRG4, NLGN4X, PCDH7, SLC27A6, SLC35F3, SLC6A2, SNAP91, SORCS1, SYT9, TM6SF1, TMEM130, TRHDE, UNC5C, and VIPR2.

Typically, the methylation status is determined in suitable CpG islands, or other CpG-rich regions, which are often found in the promoter, and enhancer, region of the gene(s). The term “methylation”, “methylation state”, “methylation level”, or “methylation status” refers to the presence or absence of 5-methylcytosine (“5-mCyt”) at one or a plurality of CpG dinucleotides within a DNA sequence, CpG dinucleotides are typically concentrated in the promoter regions and exons of human genes.

A reference methylation value or predetermined reference methylation value is for instance a value of 0.2 or more, such as 0.5 or more on the Infinium platform output. The value may also be determined with a suitable probe set, such as for instance probes comprising a sequence according to SEQ ID NO: 9, SEQ ID NO: 12, or SEQ ID NO: 15 for ITGA4, ADHFE1, and LONRF2 respectively. Probes and their sequences suitable for each of their corresponding genes ADHFE1, AMPH, FAM184B, FAM19A4, FBLIM1, GALNT13, GDNF, GFRA1, HAND2, ITGA4, KCNIP4, LONRF2, LRRC7, MARCHF11, NALCN, NDRG4, NLGN4X, PCDH7, SLC27A6, SLC35F3, SLC6A2, SNAP91, SORCS1, SYT9, TM6SF1, TMEM130, TRHDE, UNC5C, and VIPR2 are listed in Table 10.

When used in connection with the methylation of a promoter (or enhancer) region, the predetermined reference methylation level refers to the promoter (or enhancer) methylation level (PML) of the gene under the control of that promoter (or enhancer) in a sample from a subject not having CRC.

The reference value may also be determined empirically by the skilled person. The skilled person is well aware of the ins and outs of determining reference values for measuring and determining the level of their promoter methylation.

Herein, we employed two different, particularly suitable methods wherein the promoter methylation level of the genes ITGA4, ADHFE1, and LONRF2 was determined (examples 1 and 2). The promoter methylation level (PML) is inversely correlated with the expression level of the gene, i.e. the higher the PML, the lower the expression level of the gene. Table 2 shows a comparison of the sensitivity and specificity of the disclosed methods versus the commercially available Cologuard assay.

TABLE 2
Sensitivity of the disclosed methods versus
a commercial assay 10 (Cologuard)

| | Sensitivity | Specificity |
|---|---|---|
| Cologuard [1] | 92.3% | 89.9% |
| Method of the invention | 88.9% | 100% |

This means that the disclosed methods essentially detect as many patients with colon cancer as the best commercial assay available, whereas it produces far less false positive results. The method of the invention therefore results in an improvement of the Youden J statistic from 0.822 for Cologuard to 0.889.

More in detail: stool samples were obtained from 18 patients with colorectal cancer (CRC) and DNA was isolated and analyzed according to the procedures as described in example 1. Therein, the promoter methylation level (PML) of the promoters of genes ITGA4, ADHFE1, and LONRF2 was determined. If the PML of one of the promoters of a gene was above a predetermined reference value, this was interpreted as that the expression of the gene was decreased and the test was scored as positive. The results of the analysis are shown below in Table 3.

TABLE 3
DNA methylation of promoter regions of genes ITGA4,
ADHF1and LONRF2 in 18 patients with CRC.

| Patient | ADHFE1 | | ITGA4 | | LONRF | |
|---|---|---|---|---|---|---|
| # | PML | cut-off | PML | cut-off | PML | cut-off |
| 1 | 35.22 | 0.16 | 25.84 | 0.09 | 67.25 | 7.54 |
| 2 | 0.00 | 0.16 | 0.00 | 0.09 | 0.00 | 7.54 |
| 3 | 14.47 | 0.16 | 4.49 | 0.09 | 22.54 | 7.54 |
| 4 | 0.00 | 0.16 | 0.17 | 0.09 | 0.00 | 7.54 |
| 5 | 9.14 | 0.16 | 2.81 | 0.09 | 6.57 | 7.54 |
| 6 | 24.20 | 0.16 | 59.99 | 0.09 | 100.23 | 7.54 |
| 7 | 16.82 | 0.16 | 13.80 | 0.09 | 73.55 | 7.54 |
| 8 | 6.23 | 0.16 | 5.68 | 0.09 | 9.47 | 7.54 |
| 9 | 0.97 | 0.16 | 4.36 | 0.09 | 8.36 | 7.54 |
| 10 | 1.84 | 0.16 | 0.83 | 0.09 | 5.71 | 7.54 |
| 11 | 7.38 | 0.16 | 6.25 | 0.09 | 5.27 | 7.54 |
| 12 | 0.00 | 0.16 | 0.00 | 0.09 | 13.76 | 7.54 |
| 13 | 9.57 | 0.16 | 4.81 | 0.09 | 22.44 | 7.54 |
| 14 | 0.00 | 0.16 | 0.00 | 0.09 | 11.94 | 7.54 |
| 15 | 0.00 | 0.16 | 0.00 | 0.09 | 3.9 | 7.54 |
| 16 | 0.32 | 0.16 | 2.47 | 0.09 | 17.54 | 7.54 |
| 17 | 66.98 | 0.16 | 21.90 | 0.09 | 0.00 | 7.54 |
| 18 | 0.97 | 0.16 | 0.65 | 0.09 | 5.00 | 7.54 |

The same results are shown in Table 4, scored as “+” or “—” relative to the cut-off value.

TABLE 4
Results from Table 3 scored as positive (+) or negative
(−) relative to the predetermined reference value or cut-
off value. Samples were scored positive if the PML of at least
one/two/three of the three markers was above the cut-off value.

| Patient | | | | Sensitivity | | |
|---|---|---|---|---|---|---|
| # | ADHFE1 | ITGA4 | LONRF2 | 1/3 | 2/3 | 3/3 |
| 1 | + | + | + | + | + | + |
| 2 | − | − | − | − | − | − |
| 3 | + | + | + | + | + | + |
| 4 | − | + | − | + | − | − |
| 5 | + | + | − | + | + | − |
| 6 | + | + | + | + | + | + |
| 7 | + | + | + | + | + | + |
| 8 | + | + | + | + | + | + |
| 9 | + | + | + | + | + | + |
| 10 | + | + | − | + | + | − |
| 11 | + | + | − | + | + | − |
| 12 | − | − | + | + | − | − |
| 13 | + | + | + | + | + | + |
| 14 | − | − | + | + | − | − |
| 15 | − | − | − | − | − | − |
| 16 | + | + | + | + | + | + |
| 17 | + | + | − | + | + | − |
| 18 | + | + | − | + | + | − |
| False | 6/18 = | 4/18 = | 9/18 = | 2/18 = | 5/18 = | 10/18 = |
| Negative | 33% | 22% | 50% | 11.1% | 27.8% | 55.6% |
| rate | | | | | | |

Specificity of the disclosed methods was determined in a panel of 10 samples from 10 normal individuals, not having CRC. All normal subjects were found negative for any colon abnormality in colonoscopy. Their DNA was isolated from their stool and analyzed according to the procedures as described in example 1. The results of the analysis are shown herein below in Table 5.

TABLE 5
Methylation of promoter regions of genes ADHFE1,
ITGA4, and LONRF2 in 10 normal individuals.

| Patient | ADHFE1 | | ITGA4 | | LONRF2 | |
|---|---|---|---|---|---|---|
| # | PML | cut-off | PML | cut-off | PML | cut-off |
| 1 | 0.00 | 0.16 | 0.00 | 0.09 | 6.25 | 7.54 |
| 2 | 0.00 | 0.16 | 0.00 | 0.09 | 2.86 | 7.54 |
| 3 | 0.00 | 0.16 | 0.00 | 0.09 | 0.00 | 7.54 |
| 4 | 0.00 | 0.16 | 0.00 | 0.09 | 0.00 | 7.54 |
| 5 | 0.00 | 0.16 | 0.00 | 0.09 | 6.73 | 7.54 |
| 6 | 0.00 | 0.16 | 0.00 | 0.09 | 0.00 | 7.54 |
| 7 | 0.00 | 0.16 | 0.00 | 0.09 | 0.00 | 7.54 |
| 8 | 0.00 | 0.16 | 0.00 | 0.09 | 0.00 | 7.54 |
| 9 | 0.00 | 0.16 | 0.00 | 0.09 | 0.00 | 7.54 |
| 10 | 0.00 | 0.16 | 0.00 | 0.09 | 0.00 | 7.54 |

TABLE 6
Results from Table 5 scored as positive (+) or negative
(−) relative to the cut-off value. All samples were
negative for all 3 markers (sum).

| Patient # | ADHFE1 | ITGA4 | LONRF2 | Sum |
|---|---|---|---|---|
| 1 | − | − | − | − |
| 2 | − | − | − | − |
| 3 | − | − | − | − |
| 4 | − | − | − | − |
| 5 | − | − | − | − |
| 6 | − | − | − | − |
| 7 | − | − | − | − |
| 8 | − | − | − | − |
| 9 | − | − | − | − |
| 10 | − | − | − | − |
| False positive rate | 0% | 0% | 0% | 0% |

It may be concluded from these results that the disclosed methods have a specificity of 100%.

The results provided herein should not be interpreted so narrowly as to mean that the disclosed methods are restricted to the use of a particular technique to determine the expression status of the above mentioned marker genes, or even restricted to the particular set of DNA probes or primers used to determine the methylation levels of their promoters. The specific probes and primers as exemplified herein are merely provided as an example of the versatility of the method, and are by no means intended to limit the scope of the claimed subject matter.

As is shown herein below, the methylation level of the promoter regions of genes ADHFE1, ITGA4, and LONRF2 may be determined using different techniques at different CpG methylation sites within one or more promoter (or enhancer) regions.

The promoter regions of the genes ITGA4, ADHFE1, and LONRF2 are herein provided as SEQ ID NO: 1, SEQ ID NO: 3, and SEQ ID NO: 5 respectively. Suitable regions within these promoter regions for Q-PCR analysis may include but are not limited to the regions defined by SEQ ID NO: 2, SEQ ID NO: 4, and SEQ ID NO: 6. Promoter regions of the genes TRHDE, PCDH7, GALNT13, SLC27A6, TMEM130, VIPR2, FAM19A4, SLC6A2, AMPH, FBLIM1, ADHFE1, NALCN, SLC35F3, FAM184B, KCNIP4, LRRC7, TM6SF1, SYT9, NLGN4X, MARCHF11, LONRF2, and NDRG4 are herein provided as SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67 and SEQ ID NO: 68 respectively. Suitable regions within these promoter regions for Q-PCR analysis may include but are not limited to the regions defined by SEQ ID NO: 25; SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, and SEQ ID NO: 46 respectively.

We herein describe three additional probes for each of the three genes ITGA4, ADHFE1, and LONRF2 (9 probes in total) that were advantageously used for determining the promoter methylation level of the genes (Example 2).

The use of each of these probes in any combination resulted in a particularly useful method for detecting CRC. Not only was an excellent specificity achieved (100%), the sensitivity of the method as exemplified in Example 2 varied between 96% and 97%.

The highest sensitivity was achieved for the disclosed methods when a positive score was based on a promoter methylation level of one gene promoter above the cut-off value (column marked “sensitivity 1/3” in Table 11). Even when a result was scored as positive when two or three out of the three markers were positive (above a predetermined reference value or cut-off value), the sensitivity of the assay still had clinical utility (Table 11).

In addition to the 10 genes tested as described in example 1 (LONRF2, ADHFE1, ITGA4, UNC5C, GFRA1, SORCS1, SNAP91, SLC27A6, HAND2, GDNF), 19 top ranked CRC methylation biomarkers from Table 1 were evaluated in stool samples from 49 patients that were cancer-free based on the colonoscopy results, 25 patients with adenoma, 17 patients with high-grade adenoma, and 42 patients with confirmed colorectal cancer (Example 2). SLC27A6, ADHFE1, and LONRF2 were included for reference performance, totaling 22 genes.

These markers were assessed with either the delta Ct method, using a control gene as reference, or based on the detectable copy numbers as determined by a standard curve generated with known quantities of the target genes. Performance is expressed in terms of AUC (area under the ROC curve), with the individual biomarkers' performance for patients with CRC tumors, with any type of CRC precursor stage (adenoma), with high-risk CRC precursor stage (high-grade adenoma), or with high-risk CRC precursor stage or CRC versus patients without CRC tumors or precursor stages listed below, ranked according to their performance in high-grade adenomas and CRC (Table 7).

TABLE 7
Performance (AUC) of 22 DNA methylation markers
in the detection of CRC and/or precursor stages,
as assessed by the delta Ct method.

| | | | | High-grade |
|---|---|---|---|---|
| | | | High-grade | adenomas |
| marker | CRC | Adenomas | adenomas | and CRC |
| SLC35F3 | 0.72 | 0.57 | 0.66 | 0.71 |
| LONRF2 | 0.71 | 0.51 | 0.60 | 0.68 |
| FBLIM1 | 0.71 | 0.52 | 0.54 | 0.66 |
| SLC27A6 | 0.67 | 0.57 | 0.55 | 0.64 |
| TM6SF1 | 0.63 | 0.58 | 0.63 | 0.63 |
| FAM184B | 0.63 | 0.49 | 0.58 | 0.62 |
| NDRG4 | 0.68 | 0.51 | 0.59 | 0.62 |
| TMEM130 | 0.62 | 0.56 | 0.60 | 0.61 |
| SYT9 | 0.62 | 0.53 | 0.60 | 0.61 |
| AMPH | 0.58 | 0.45 | 0.65 | 0.60 |
| GALNT13 | 0.61 | 0.55 | 0.55 | 0.60 |
| MARCHF11 | 0.59 | 0.55 | 0.59 | 0.59 |
| SLC6A2 | 0.63 | 0.51 | 0.51 | 0.59 |
| TRHDE | 0.61 | 0.52 | 0.49 | 0.58 |
| VIPR2 | 0.56 | 0.59 | 0.60 | 0.57 |
| KCNIP4 | 0.56 | 0.50 | 0.58 | 0.56 |
| PCDH7 | 0.53 | 0.49 | 0.49 | 0.53 |
| LRRC7 | 0.49 | 0.50 | 0.65 | 0.53 |
| NLGN4X | 0.50 | 0.48 | 0.42 | 0.52 |
| ADHFE1 | 0.56 | 0.53 | 0.65 | 0.51 |
| NALCN | 0.54 | 0.53 | 0.56 | 0.51 |
| FAM19A4 | 0.53 | 0.54 | 0.55 | 0.49 |

An analogous table can be created for the copy number analysis (Table 8).

TABLE 8
Performance (AUC) of 22 DNA methylation markers
in the detection of CRC and/or precursor stages,
as assessed by the copy number method.

| | | | | High-grade |
|---|---|---|---|---|
| | | | High-grade | adenomas |
| marker | CRC | Adenomas | adenomas | and CRC |
| SLC35F3 | 0.79 | 0.57 | 0.67 | 0.76 |
| FAM184B | 0.71 | 0.49 | 0.59 | 0.68 |
| TM6SF1 | 0.68 | 0.58 | 0.63 | 0.67 |
| SYT9 | 0.68 | 0.52 | 0.62 | 0.66 |
| AMPH | 0.66 | 0.45 | 0.65 | 0.65 |
| MARCHF11 | 0.66 | 0.55 | 0.60 | 0.65 |
| SLC27A6 | 0.68 | 0.54 | 0.54 | 0.64 |
| TMEM130 | 0.63 | 0.58 | 0.63 | 0.63 |
| KCNIP4 | 0.64 | 0.51 | 0.59 | 0.63 |
| FBLIM1 | 0.67 | 0.55 | 0.56 | 0.62 |
| GALNT13 | 0.62 | 0.56 | 0.58 | 0.61 |
| SLC6A2 | 0.64 | 0.47 | 0.48 | 0.61 |
| VIPR2 | 0.58 | 0.61 | 0.60 | 0.59 |
| LRRC7 | 0.56 | 0.52 | 0.62 | 0.58 |
| NLGN4X | 0.58 | 0.48 | 0.57 | 0.58 |
| ADHFE1 | 0.54 | 0.55 | 0.67 | 0.57 |
| FAM19A4 | 0.59 | 0.55 | 0.55 | 0.55 |
| PCDH7 | 0.55 | 0.50 | 0.51 | 0.54 |
| NALCN | 0.52 | 0.53 | 0.57 | 0.53 |
| TRHDE | 0.54 | 0.49 | 0.50 | 0.53 |
| LONRF2 | 0.61 | 0.57 | 0.60 | 0.53 |

All markers have a positive effect on the specific detection of CRC or CRC precursor stages and hence assessing all these markers simultaneously could be advantageous. However, since that might not be (technically) achievable or might require a too big of a dilution of the sample, it is investigated which genes are most complementary in terms of performance. Genes are scored based on their relative importance when creating any possible panel of 3 genes.

All possible 1540 combinations of panels of 3 genes for the delta Ct method were scored in terms of AUC by fitting a logistic regression model for the detection of CRC, any type of adenoma, high-grade adenoma, and the combination of high-grade adenoma. The controls remain the same for each one of these, i.e. patients with no sign of adenoma or CRC. These four comparisons were supplemented with the addition of FIT (fecal immunochemical test) as biomarker for CRC and high-risk adenoma's, given a total of 8 AUC values per panel of 3 genes.

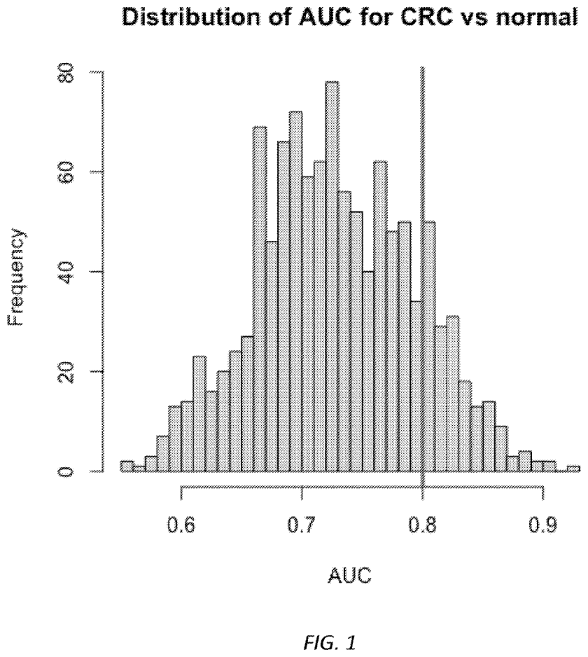

Only panels with AUC values indicating better performance than what would be randomly expected were retained, i.e. AUC>0.55, leaving 1120 panels. Next the panels were scored to assess their overall performance, i.e. for CRC, adenoma, high-grade adenoma, and the combination of high-grade adenoma and CRC, with and without the addition of FIT. To this purpose, the distribution of AUC values for each one of the 8 individual comparisons was inspected and a cutoff value, clearly enriching for the most performing panels, was identified. This cutoff was subtracted from the actual AUC of each one of the panels, hence with panels having an AUC above this cutoff value being scored positively, and panels below this cutoff being scored negatively. An example is shown in FIG. 1.

Finally, these scores for all 8 individual AUC comparisons were added, and only panels that scored overall positively (>0) were retained, resulting in 204 panels. All 22 genes were part of these panels, again indicating that all can contribute to an accurate detection of CRC and CRC precursor stages. To assess the importance of each individual gene, especially in light of complementarity to other markers and to other markers in combination with FIT, the panels were ordered according to their decreasing score. The rank of each gene in this ordered table was determined for 6 comparisons. The ranks for high-grade adenoma in combination with CRC (with and without FIT) were not included, since these are already reflected in the rank for CRC on the one hand and the rank for high-grade adenoma on the other hand. All of these ranks were summed together, with lower summed ranks indicating a higher importance for the individual gene (and the theoretical minimal rank being 6, i.e. positioned as first for all 6 comparisons). The resulted in the following ranking for the 22 genes: FBLIM1, SLC35F3, TM6SF1, NDRG4, SLC6A2, LONRF2, SLC27A6, FAM19A4, VIPR2, KCNIP4, MARCHF11, TMEM130, TRHDE, AMPH, ADHFE1, LRRC7, NALCN, GALNT13, SYT9, PCDH7, NLGN4X, and FAM184B. While building larger panels including as many genes as possible from this list would be beneficial, the above ranking assigns a priority to the genes for smaller panels.

The same methodology, using the same cutoff values was applied to the data generated with the copy number method, resulting in the ranking below (since no plasmid was available for NDRG4, no copy numbers were determined for this gene): SLC35F3, TM6SF1, LONRF2, SLC27A6, SLC6A2, FBLIM1, KCNIP4, VIPR2, FAM19A4, TMEM130, ADHFE1, MARCHF11, AMPH, TRHDE, LRRC7, GALNT13, NALCN, PCDH7, SYT9, NLGN4X, and FAM184B.

A ranking of the genes is determined by combining both the delta Ct method rank and the copy number rank: SLC35F3, TM6SF1, FBLIM1, LONRF2, SLC27A6, SLC6A2, KCNIP4, FAM19A4, VIPR2, TMEM130, MARCHF11, ADHFE1, AMPH, TRHDE, LRRC7, GALNT13, NALCN, SYT9, PCDH7, NLGN4X, FAM184B, and NDRG4.

Finally, it was evaluated whether methylated copies of these genes could be identified in 23 blood samples from CRC patients (example 4). The presence of detectable copies of any gene of NLGN4X, NALCN, TMEM130, FAM19A4, TM6SF1, AMPH, PCDH7, SLC27A6, SLC6A2, TRHDE, VIPR2, LRCC7, COL25A1, FAM184B, FBLIM1, GALNT13, KCNIP4, SLC35F3, SYT9, SNAP91, ITGA4, ADHFE1, and NDRG4. 18 out of 23 (78%) of the blood samples of these CRC patients had detectable copies for at least 1 of these 23 genes.

The disclosed methods may be performed on any sample taken from a subject; however, it is advantageous to use stool samples because they are easily obtainable. Tissue samples, for instance from a colon biopsy may also be advantageously employed because they contain a relatively large amount of tumor DNA. Other types of samples may be used in the disclosed methods. Non-limiting examples include human or animal fresh tissue samples, frozen tissue samples, tissue samples embedded in FFPE (formalin-fixed, paraffin-embedded tissue), whole blood, blood plasma, blood serum, urine, stool, lymph fluid, and rectal swabs. The samples may be collected using any suitable methods known in the art.

As discussed, the sample may be a tissue sample, swab specimen, body fluid, body fluid precipitate or lavage specimen. Typically, the sample under investigation contains nucleic acid from a colorectal tumor or colorectal cancer to be analyzed according to the disclosed methods.

The disclosed subject matter also provides systems and kits for performing the disclosed methods. In particular, the disclosed subject matter provides systems and kits for detecting alterations in expression status in a nucleic acid containing colorectal tumor or colorectal cancer sample, the systems and kits comprising: components, reagents, and/or tools to specifically detect methylation levels or promotor methylation levels of at least one of the genes listed in Table 1.

The disclosed components, reagents, and/or tools may be sequence-specific and/or may be utilized for detecting expression of at least one gene listed in Table 1 in a sequence specific-manner. The disclosed components, reagents, and/or tools may be utilized for detecting promotor methylation of at least one gene listed in Table 1. The disclosed components, reagents, and/or tools may include, but are not limited to primers, probes, and other reagents such as a proofreading polymerase. In some embodiments, primers and probes suitable for the detection of colorectal cancer according to the disclosed methods, systems, and kits are disclosed in the description and comprise: a probe according to SEQ ID NO: 9 and primers according to SEQ ID NO: 7 and SEQ ID NO: 8 for measuring the methylation level of the promoter of gene ITGA4; a probe according to SEQ ID NO: 12 and primers according to SEQ ID NO: 10 and SEQ ID NO: 11 for measuring the methylation level of the promoter of gene ADHFE1; and a probe according to SEQ ID NO: 15 and primers according to SEQ ID NO: 13 and SEQ ID NO: 14 for measuring the methylation level of the promoter of gene LONRF2; a probe according to SEQ ID NO: 113 and primers according to SEQ ID NO: 69 and SEQ ID NO: 91 for measuring the methylation level of the promoter of gene TRHDE; a probe according to SEQ ID NO: 114 and primers according to SEQ ID NO: 70 and SEQ ID NO: 92 for measuring the methylation level of the promoter of gene PCDH7; a probe according to SEQ ID NO: 115 and primers according to SEQ ID NO: 71 and SEQ ID NO: 93 for measuring the methylation level of the promoter of gene GALNT13; a probe according to SEQ ID NO: 116 and primers according to SEQ ID NO: 72 and SEQ ID NO: 94 for measuring the methylation level of the promoter of gene SLC27A6; a probe according to SEQ ID NO: 117 and primers according to SEQ ID NO: 73 and SEQ ID NO: 95 for measuring the methylation level of the promoter of gene TMEM130; a probe according to SEQ ID NO: 118 and primers according to SEQ ID NO: 74 and SEQ ID NO: 96 for measuring the methylation level of the promoter of gene VIPR2; a probe according to SEQ ID NO: 119 and primers according to SEQ ID NO: 75 and SEQ ID NO: 97 for measuring the methylation level of the promoter of gene FAM19A4; a probe according to SEQ ID NO: 120 and primers according to SEQ ID NO: 76 and SEQ ID NO: 98 for measuring the methylation level of the promoter of gene SLC6A2; a probe according to SEQ ID NO: 121 and primers according to SEQ ID NO: 77 and SEQ ID NO: 99 for measuring the methylation level of the promoter of gene AMPH; a probe according to SEQ ID NO: 122 and primers according to SEQ ID NO: 78 and SEQ ID NO: 100 for measuring the methylation level of the promoter of gene FBLIM1; a probe according to SEQ ID NO: 123 and primers according to SEQ ID NO: 79 and SEQ ID NO: 101 for measuring the methylation level of the promoter of gene COL25A1; a probe according to SEQ ID NO: 124 and primers according to SEQ ID NO: 80 and SEQ ID NO: 102 for measuring the methylation level of the promoter of gene NALCN; a probe according to SEQ ID NO: 125 and primers according to SEQ ID NO: 81 and SEQ ID NO: 103 for measuring the methylation level of the promoter of gene SLC35F3; a probe according to SEQ ID NO: 126 and primers according to SEQ ID NO: 82 and SEQ ID NO: 104 for measuring the methylation level of the promoter of gene FAM184B; a probe according to SEQ ID NO: 127 and primers according to SEQ ID NO: 83 and SEQ ID NO: 105 for measuring the methylation level of the promoter of gene KCNIP4; a probe according to SEQ ID NO: 128 and primers according to SEQ ID NO: 84 and SEQ ID NO: 106 for measuring the methylation level of the promoter of gene LRRC7; a probe according to SEQ ID NO: 129 and primers according to SEQ ID NO: 85 and SEQ ID NO: 107 for measuring the methylation level of the promoter of gene TM6SF1; a probe according to SEQ ID NO: 130 and primers according to SEQ ID NO: 86 and SEQ ID NO: 108 for measuring the methylation level of the promoter of gene SYT9; a probe according to SEQ ID NO: 131 and primers according to SEQ ID NO: 87 and SEQ ID NO: 109 for measuring the methylation level of the promoter of gene NLGN4X; a probe according to SEQ ID NO: 132 and primers according to SEQ ID NO: 88 and SEQ ID NO: 110 for measuring the methylation level of the promoter of gene MARCHF11; a probe according to SEQ ID NO: 133 and primers according to SEQ ID NO: 89 and SEQ ID NO: 111 for measuring the methylation level of the promoter of gene LONRF2; and a probe according to SEQ ID NO: 134 and primers according to SEQ ID NO: 90 and SEQ ID NO: 112 for measuring the methylation level of the promoter of gene NDRG4. The systems and kits may also comprise control biological sample material.

The disclosed systems and kits may comprise one or more components, reagents, and/or tools for detecting the expression of at least one gene listed in Table 1. In some embodiments, the disclosed systems and kits comprise one or more components, reagents, and/or tools for detecting the methylation status of at least one gene listed in Table 1, and optionally for detecting the methylation status of multiple genes listed in Table 1. Suitable components, reagents, and/or tools for detecting the methylation status of at least one gene listed in Table 1 may include, but are not limited to, components, reagents, and/or tools for performing one or more of the following processes: PCR, Q-PCR, sequencing, hybridization arrays, restriction enzyme approaches (e.g, methylation-specific PCR (MSP), quantitative methylation-specific PCR (Q-MSP), Illumina sequencing technology, Oxford Nanopore sequencing technology, Ion Torrent™ sequencing technology, Dreaming technology, and High Resolution Melting (HRM) technology). Suitable components, reagents, and/or tools may include, but are not limited to reagents that convert unmethylated cytosines to uracil but that do not convert methylated cytosines to uracil (e.g., bisulfite reagents); sequence-specific primers for performing PCR of at least one gene listed in Table 1, either before or after the gene is treated with a reagent that converts unmethylated cytosines to uracil but that does not convert methylated cytosines to uracil (e.g., bisulfite reagents); sequence-specific probes for detecting at least one gene listed in Table 1 and/or a PCR product thereof (e.g., an amplicon thereof) either before or after the gene or the PCR product thereof is treated with a reagent that converts unmethylated cytosines to uracil but that does not convert methylated cytosines to uracil; primers for sequencing a gene listed in Table 1 or a PCR product thereof (e.g., an amplicon thereof) either before or after the gene or the PCR product thereof is treated with a reagent that converts unmethylated cytosines to uracil but that does not convert methylated cytosines to uracil; enzymes for performing PCR amplification; enzymes for cleaving a nucleic acid target based on the methylation status of the nucleic acid target (e.g., restriction enzymes); and other reagents for performing any of the foregoing reactions (e.g. buffers, nucleotides, and cations (e.g., Mg++)).

The disclosed systems may be designed for detecting the expression status or the methylation status of at least one gene listed in Table 1 in a biological sample from a subject, and optionally designed for detecting the expression status or the methylation status of multiple genes listed in Table 1 in a biological sample from a subject. In some embodiments, the disclosed systems may comprise at least one hardware processor that is programmed to perform at least one step for detecting the expression status or the methylation status of at least one gene listed in Table 1 in a biological sample from a subject and/or a hardware processor that is programmed to actuate at least one mechanical component of the system to perform one or more steps for detecting the expression status or the methylation status of multiple genes listed in Table 1 in a biological sample from a subject. In some embodiments, the hardware processor may be configured to perform and/or to actuate one or more mechanical components of the system to perform one or more of the following tasks: (i) receiving and/or transporting a biological sample from a subject into the system; (ii) adding one or more components, reagents, and/or tools to the biological sample (e.g., one or more components, reagents, and/or tools to perform demethylation and/or PCR and/or sequencing of at least one of the genes listed in Table 1); (iii) performing PCR on the biological sample or a demethylated product thereof; (iv) detecting a PCR product (e.g., a PCR product of one or more of the genes listed in Table 1 or a demethylated product thereof; (v) sequencing at least one of the genes listed in Table 1 in the biological sample or a demethylated product thereof; (vi) generating a report that indicates the methylation status of one or more genes listed in Table 1 and/or the expression status? of one or more genes listed in Table 1, optionally relative to a control.

The disclosed systems may include automated systems. The disclosed kits may be in any format, including a format suitable for automated systems, such as a cartridge format. Such systems have been described and are well known to the skilled person. Typically, cartridges for automated systems for detecting methylation analysis of at least one gene are preloaded with reagents and the analysis of promotor methylation is performed in an automated manner by means of a computer-implemented method by an instrument. Once the sample is added to the cartridge, the automated system will run the assay.

The detection of methylation or promotor methylation may utilize a biological sample comprising nucleic acids or cells comprising nucleic acid to be analyzed according to the disclosed methods. Methods and systems for obtaining nucleic acid from samples have been described and may require isolation and/or purification of the nucleic acid from the sample.

Based on the present disclosure, the skilled person will be able to design further primers and probe sets for Q-PCR or probes for DNA arrays that will detect at least one methylated CpG dinucleotide in the promoter regions of the genes as disclosed herein.

Detection of promoter methylation may also be determined in a number of alternative ways as the ones exemplified herein. This methylation level may be determined by a method selected from the group consisting of PCR, Q-PCR, sequencing, hybridization arrays, restriction enzyme approaches, such as for instance methylation-specific PCR (MSP), quantitative methylation-specific PCR (Q-MSP), Illumina sequencing technology, Oxford Nanopore sequencing technology, Ion Torrent™ sequencing technology, Dreaming technology, High Resolution Melting (HRM) technology, and the like.

The detection method DREAMing (Discrimination of Rare EpiAlleles by Melt) technology uses semi-limiting dilutions and precise melt curve analysis to distinguish and enumerate individual copies of epi-allelic species at single-CpG-site resolution in fractions as low as 0.005%, providing facile and inexpensive ultrasensitive assessment of locus-specific epigenetic heterogeneity directly from liquid biopsies such as stool, urine, and blood samples [6].

The disclosed methods may include further steps such as diagnostic steps and treatment steps. In some embodiments, the disclosed methods comprise performing a colon biopsy on the subject. In some embodiments, the disclosed methods may comprise administering to the subject a treatment for colorectal cancer, optionally selected from a chemotherapy, a biological agent, a surgery, and/or radiation therapy.

The skilled person is familiar with these techniques and will not experience any difficulties in applying them.

### EXAMPLES

### Example 1: DNA Isolation, Bisulfite Conversion, and Promoter CpG Island Methylation Analyses

DNA was isolated from stool samples. Buffered whole stool samples (35 ml) were centrifuged to remove insoluble parts. DNA was precipitated using ethanol, washed, and dissolved. DNA was isolated using the Qiagen QIAamp Fast DNA stool kit.

Next, sodium bisulfite modification of 500 ng genomic DNA was performed using the EpiTect Bisulfite Kit (Qiagen, Venlo, The Netherlands) according to the manufacturer's instructions.

Following bisulfite conversion, quantitative methylation-specific polymerase chain reaction (Q-MSP) analyses were performed as described elsewhere [5]. Primer and probe sequences are shown in Table 9. Q-MSP data were analysed using the PML-method (promoter methylation level). All cut-offs were determined using ROC analysis. Samples were scored as positive when PML of ITGA4 was above 0.09, PML of ADHFE1 was above 0.16, or PML of LONRF2 was above 7.54.

Q-PCR conditions were as follows: The PCR mixture contains 1×PCR buffer (16.6 mM ammonium sulfate/67 mM Tris, pH 8.8/6.7 mM MgCl2/10 mM 2-mercaptoethanol), dNTPs (each at 1.25 mM), primers (300 ng each per reaction), and bisulfite-modified DNA (50 ng) in a final volume of 50 ul. Reactions were hot-started at 95° C. for 5 min before the addition of 1.25 units of Taq polymerase (BRL). Amplification was carried out in a thermocycler for 50 cycles (30 sec at 95° C., 30 sec at the annealing temperature of 64° C., and 30 sec at 72° C.), followed by a final 4-min extension at 72° C.

Q-PCR reactions were performed with controls for unmethylated alleles (for example unmethylated human control DNA, EpiTect Control DNA, Qiagen, Cat. no. 59568), methylated alleles (normal lymphocyte DNA treated in vitro with SssI methyltransferase [New England Biolabs]), and a no-template DNA control.

TABLE 9
Probes and primers used for Q PCR.

| Primer/Probe | Sequence | SEQ ID NO: |
|---|---|---|
| ITGA4 Forward | GGGATTTTATTTAGCGGTTCGTATTC | 7 |
| ITGA4 Reverse | GAACCGACCTAAAATACCGCG | 8 |
| ITGA4 Probe | TTGGCGGTAGAAATCGGGAGTGGGGTC | 9 |
| ADHFE Forward | TTTGAGGTTTAGATAGGTGATTTCGC | 10 |
| ADHFE1 Reverse | CGACCAATCACGAAAACTACCCG | 11 |
| ADHFE1 Probe | CGGGTGGGTAGGCGCGGTC | 12 |
| LONRF2 Forward | CGGCGGGATTGAGAGGTC | 13 |
| LONRF2 Reverse | ACCGAAACAAACACCGCG | 14 |
| LONRF2 Probe | CGCGTGGGTAGGGGTTTAGATTGCGT | 15 |

### Example 2: DNA Isolation, Bisulfite Conversion, and Promoter CpG Island Methylation Analyses

DNA was isolated from stool samples. Buffered whole stool samples (4-10 ml) were centrifuged to remove insoluble parts. DNA was precipitated using ethanol, washed, and dissolved. DNA was isolated using the Qiagen QIAamp Fast DNA stool kit.

Next, sodium bisulfite modification of 500 ng genomic DNA was performed using the Zymo Lightning Bisulfite Kit (Zymo Reseach, BaseClear, The Netherlands) according to the manufacturer's instructions.

Following bisulfite conversion, quantitative methylation-specific polymerase chain reaction (Q-MSP) analyses were performed as described elsewhere [5]. Primer and probe sequences are shown in Table 10. Q-MSP data were analysed using the PML-method (promoter methylation level). The DNA methylation level of each marker was determined by calculating normalized delta Ct values (difference in Ct between marker and a neutral assay) and normalized copy numbers (based on a standard curve and comparison with a neutral assay).

Q-PCR conditions were as follows: The PCR mixture contains 2x PCR mix (PrimeTime Gene expression Master mix), primers (375 nM each per reaction), probe (250 nM per.reaction), and bisulfite-modified DNA (10 ng) in a final volume of 20 ul. Reactions were performed on a LightCycler 480 (Roche). Polymerase activation for 3 min at 95° C. Amplification was carried out for 45 cycles (15 sec at 95° C. and 60 sec at the annealing temperature of 65° C.).

Q-PCR reactions were performed with controls for unmethylated alleles (for example Double knock out of the HCT-116 human cell line), methylated alleles (CpG Methylated Human Genomic DNA), and a no-template DNA control.

TABLE 10
Probes and primers used in Example 2.

| | | | | Amplicon |
|---|---|---|---|---|
| Marker | Forward primer | Reverse primer | Probe | size (bp) |
| TRHDE | GCGGAAGTTGTCGTCG (SEQ ID NO. | CCACAACCAAAACAAACATCG | AGTCGCGCGTTTTCGGTTCGC (SEQ ID NO 113) | 113 |
| | 69) | (SEQ ID NO 91) | | |
| PCDH7 | CGTTCGTATCGGTAACGTG (SEQ ID | GCTCGTACTCAACTCGC (SEQ ID NO | ATCGTGATCGGATCGGGTGAGGTG (SEQ ID NO 114) | 127 |
| | NO 70) | 92) | | |
| GALNT13 | GCGTTTTTCGGCGAGAG (SEQ ID NO | GCTCCAACTACTTCTAAAACTAACG | TGTTAAGTATCGCGGGATTTGCGTATATCGTAGA (SEQ | 113 |
| | 71) | (SEQ ID NO 93) | ID NO 115) | |
| SLC27A6 | AAGAGTTAGGGTTTAATAAGTTTTTCG | CTCACTACGCTAAACCCG (SEQ ID | AGTTGGAGATTTCGATAGAGCGTCGGT (SEQ ID NO 116) | 146 |
| | (SEQ ID NO 72) | NO 94) | | |
| TMEM130 | CGGACGCGGAGGGAATGTAG (SEQ ID | CACGACGCCCGCACCT (SEQ ID NO | CGCGTAGCGCGGGCGGTGCG (SEQ ID NO 117) | 95 |
| | NO 73) | 95) | | |
| VIPR2 | CATAAACCGAAAACGAATACGCG | GTTGTCGGGACGGAGG (SEQ ID NO | CGCGTTCGGGCGTTCGGTTATAGTTGCG (SEQ ID NO 118) | 91 |
| | (SEQ ID NO 74) | 96) | | |
| FAM19A4 | CGCACCCCTACTCAAACGACG (SEQ ID | CGGAGTGGTGGTTATAGCGAGG | CCACTACCGACCTCCAAACGCCTCTCCCG (SEQ ID NO | 81 |
| | NO 75) | (SEQ ID NO 97) | 119) | |
| SLC6A2 | CGCTAAATTAATACAATCGACGCGT | GCGCGGGTTGATTGGT (SEQ ID NO | TCGTGTTTGGCGGGGGTCGG (SEQ ID NO 120) | 94 |
| | (SEQ ID NO 76) | 98) | | |
| AMPH | GTTGCGAAGAGTAGAGCGCG (SEQ ID | CCGCTCCTCCCTTACTCGAC (SEQ | TGGCGGCGGCGCGGAG (SEQ ID NO 121) | 98 |
| | NO 77) | ID NO 99) | | |
| FBLIM1 | ACGTTTAGTAAACGGTCGTAG (SEQ ID | CCGAATAATCCCTACCGTC (SEQ ID | AGTAAGCGTTTATTTTCGTCGGATAGGGTAGGTG (SEQ | 152 |
| | NO 78) | NO 100) | ID NO 122) | |
| COL25A1 | GAAATCGCGACTACCCG (SEQ ID NO | GCGCGCGCGTATTTATA (SEQ ID | ATCGCGTACGCGGGGTTGC (SEQ ID NO 123) | 108 |
| | 79) | NO 101) | | |
| NALCN | CGCAACCCCTCTAACAACCCCTCTCG | GCGGCGCGGTCGGTTTCG (SEQ ID | CCCAACGCCCCTAAACCGTCCTAACCCGAACCGACC | 127 |
| | (SEQ ID NO 80) | NO 102) | (SEQ ID NO 124) | |
| SLC35F3 | GATTTTCGGTGGGTAGCG (SEQ ID NO | CGCCGCTAAAAAACTCTCG (SEQ ID | CGGTTGTAGGGAGTTTCGGTTCGCG (SEQ ID NO 125) | 143 |
| | 81) | NO 103) | | |
| FAM184B | CGGGTATTGCGTTTTTGGATACG (SEQ | CCTATCCCTCTCCGAAAACCG (SEQ | CGCGCGGTTTTGGATTTGGTGGCGACG (SEQ ID NO 126) | 153 |
| | ID NO 82) | ID NO 104) | | |
| KCNIP4 | CGAAAACGAACGCCCG (SEQ ID NO | CGGTGGATTTTCGAGTTTCG (SEQ | CGGTTTAGTTGGAGGAGGTTAGTTTTATAGGCGGTAGGA | 144 |
| | 83) | ID NO 105) | (SEQ ID NO 127) | |
| LRRC7 | AGGCGGCGAAGATGTG (SEQ ID NO | CAACACCAACGCCCAACAT (SEQ ID | CGGGTGTATGGGAGTATCGAGTAGTTTGAGTTGG (SEQ | 151 |
| | 84) | NO 106) | ID NO 128) | |
| TM6SF1 | CGCGCGGAGGAGATATCG (SEQ ID NO | CAACTTCCGACGACGCC (SEQ ID | CGCGGTTGAGGCGTTTCGAAGGGTAAGCG (SEQ ID NO | 112 |
| | 85) | NO 107) | 129) | |
| SYT9 | ATTCGGCGGTGGGAGCG (SEQ ID NO | CCAAAACGACCGCGCCCTAAC | GGTGGAGTAGCGAAAGCGTTCGGAGCGCG (SEQ ID NO | 114 |
| | 86) | (SEQ ID NO 108) | 130) | |
| NLGN4X | CGAAACCCACACACACG (SEQ ID NO | GGAACGATTTAGGCGCG (SEQ ID | CGCGTGAAGATGAAATGACGGTAGTTTCG (SEQ ID NO | 118 |
| | 87) | NO 109) | 131) | |
| MARCHF11 | CGCGGATTAGTGGGACG (SEQ ID NO | GAAACCGACGTAAACGCG (SEQ ID | CGAGCGGGCGGGTTGGAAGTG (SEQ ID NO 132) | 107 |
| | 88) | NO 110) | | |
| LONRF2 | CGGCGGGATTGAGAGGTC (SEQ ID NO | ACCGAAACAAACACCGCG (SEQ ID | CGCGTGGGTAGGGGTTTAGATTGCGT (SEQ ID NO 133) | 67 |
| | 89) | NO 111) | | |
| NDRG4 | GCGTAGAAGGCGGAAGTTAC (SEQ ID | ACGAATATAAACGCTCGACCCG | AGGGATCGCGGTTCGTTCGG (SEQ ID NO 134) | 95 |
| | NO 90) | (SEQ ID NO 112) | | |

### Example 3: DNA Array Analysis of Methylation

DNA was isolated from primary CRC tissue. Formalin-fixed, paraffin-embedded slides with CRC tissue were reviewed by an experienced pathologist. Slides were de-paraffinised and DNA was extracted following macro dissection with the QIAamp DNA Mini Kit (Qiagen, Venlo, The Netherlands). We employed the Infinium 450 k microarray platform [4] for analysis of the PML of the promoters of genes ITGA1, ADHFE1, and LONRF2. Using the probes as shown in Tables 11, 12, and 13, we found that an assay as disclosed herein resulted in a specificity of 100% and a sensitivity of between 59 and 97% in a population of 299 patients with CRC and 41 normal individuals (Table 14).

TABLE 11

| | | | | Relative to chromosome | | Relative to SEQ | | | |
|---|---|---|---|---|---|---|---|---|---|
| | | | Chr | (Chr) | | ID NO: 1 | | | SEQ |
| Gene | Probe | Strand | # | Start | End | Start | End | Sequence (*) | ID NO: |
| ITGA4 | cg06952671 | + | 2 | 181457541 | 181457590 | 780 | 829 | gattaaccaa | 16 |
| | | | | | | | | cgaaaaaaaa | |
| | | | | | | | | cgccccaaaa | |
| | | | | | | | | aataaaacgc | |
| | | | | | | | | aacgtatccg | |
| ITGA4 | cg21995919 | + | 2 | 181457552 | 181457601 | 791 | 840 | cgaaatacga | 17 |
| | | | | | | | | cgattaacca | |
| | | | | | | | | acgaaaaaaa | |
| | | | | | | | | acgccccaaa | |
| | | | | | | | | aaataaaacg | |
| ITGA4 | cg25024074 | + | 2 | 181457774 | 181457823 | 1062 | 1062 | cgaccgaata | 18 |
| | | | | | | | | accgaacaac | |
| | | | | | | | | gtattataaa | |
| | | | | | | | | aaccctaata | |
| | | | | | | | | aaacaacgcg | |

TABLE 12

| | | | | Relative to | | Relative to SEQ | | | |
|---|---|---|---|---|---|---|---|---|---|
| | | | Chr | chromosome (Chr) | | ID NO: 3 | | | SEQ |
| Gene | Probe | Strand | # | Start | End | Start | End | Sequence (*) | ID NO: |
| ADHFE1 | cg01588438 | - | 8 | 66432269 | 66432318 | 872 | 921 | ctacraaaca | 19 |
| | | | | | | | | attaccttct | |
| | | | | | | | | acracaattt | |
| | | | | | | | | caaaaataaa | |
| | | | | | | | | taatacraac | |
| ADHFE1 | cg01988129 | - | 8 | 66432652 | 66432701 | 1255 | 1304 | ctacrcaatc | 20 |
| | | | | | | | | ttctccrctt | |
| | | | | | | | | actttcaaaa | |
| | | | | | | | | attcraaaaa | |
| | | | | | | | | tttaaatacc | |
| ADHFE1 | cg20295442 | - | 8 | 66432382 | 66432431 | 985 | 1034 | cttaaaactt | 21 |
| | | | | | | | | aaacaaataa | |
| | | | | | | | | ccccgcgaaa | |
| | | | | | | | | cgaataaaca | |
| | | | | | | | | aacgcgaccg | |

TABLE 13

| | | | | Relative to | | Relative to SEQ | | | |
|---|---|---|---|---|---|---|---|---|---|
| | | Strand | Ch | chromosome (Chr) | | ID NO: 5 | | | SEQ |
| Gene | Probe | | # | Start | End | Start | End | Sequence(*) | ID NO: |
| LONRF2 | cg03559235 | + | 2 | 100321482 | 100321531 | 440 | 489 | aaaaatactt | 22 |
| | | | | | | | | cccgaccgaa | |
| | | | | | | | | taccgactac | |
| | | | | | | | | gcaaactaac | |
| | | | | | | | | aaaccaaacg | |
| LONRF2 | cg07304692 | - | 2 | 100322534 | 100322583 | 1492 | 1541 | aaaaataccr | 23 |
| | | | | | | | | aaaccctaaa | |
| | | | | | | | | aacaaataac | |
| | | | | | | | | cttaaccrcr | |
| | | | | | | | | aactactaac | |
| LONRF2 | cg23977632 | - | 2 | 100322288 | 100322337 | 1247 | 1296 | atataaacga | 24 |
| | | | | | | | | cgaaccccgc | |
| | | | | | | | | aaaactacaa | |
| | | | | | | | | tcgttccgaa | |
| | | | | | | | | ataaaaaccg | |

TABLE 14
Sensitivity and specificity of an assay as disclosed herein.

| Combination | Sensitivity | Sensitivity | Sensitivity | Specificity |
|---|---|---|---|---|
| of probes | [%] (1/3) | [%] (2/3) | [%] (3/3) | [%] |
| cg06952671 + | 96 | 87 | 68 | 100 |
| cg01588438 + | | | | |
| cg03559235 | | | | |
| cg21995919 + | 96 | 84 | 64 | 100 |
| cg01588438 + | | | | |
| cg03559235 | | | | |
| cg25024074 + | 96 | 88 | 69 | 100 |
| cg01588438 + | | | | |
| cg03559235 | | | | |
| cg06952671 + | 97 | 87 | 68 | 100 |
| cg01988129 + | | | | |
| cg03559235 | | | | |
| cg21995919 + | 97 | 84 | 64 | 100 |
| cg01988129 + | | | | |
| cg03559235 | | | | |
| cg25024074 + | 97 | 88 | 69 | 100 |
| cg01988129 + | | | | |
| cg03559235 | | | | |
| cg06952671 + | 96 | 87 | 68 | 100 |
| cg20295442 + | | | | |
| cg03559235 | | | | |
| cg21995919 + | 96 | 84 | 63 | 100 |
| cg20295442 + | | | | |
| cg03559235 | | | | |
| cg25024074 + | 96 | 88 | 69 | 100 |
| cg20295442 + | | | | |
| cg03559235 | | | | |
| cg06952671 + | 96 | 86 | 67 | 100 |
| cg01588438 + | | | | |
| cg07304692 | | | | |
| cg21995919 + | 96 | 85 | 61 | 100 |
| cg01588438 + | | | | |
| cg07304692 | | | | |
| cg25024074 + | 96 | 88 | 67 | 100 |
| cg01588438 + | | | | |
| cg07304692 | | | | |
| cg06952671 + | 97 | 86 | 67 | 100 |
| cg01988129 + | | | | |
| cg07304692 | | | | |
| cg21995919 + | 97 | 85 | 61 | 100 |
| cg01988129 + | | | | |
| cg07304692 | | | | |
| cg25024074 + | 97 | 89 | 67 | 100 |
| cg01988129 + | | | | |
| cg07304692 | | | | |
| cg06952671 + | 96 | 86 | 66 | 100 |
| cg20295442 + | | | | |
| cg07304692 | | | | |
| cg21995919 + | 96 | 85 | 60 | 100 |
| cg20295442 + | | | | |
| cg07304692 | | | | |
| cg25024074 + | 96 | 88 | 67 | 100 |
| cg20295442 + | | | | |
| cg07304692 | | | | |
| cg06952671 + | 96 | 85 | 64 | 100 |
| cg01588438 + | | | | |
| cg23977631 | | | | |
| cg21995919 + | 96 | 81 | 60 | 100 |
| cg01588438 + | | | | |
| cg23977631 | | | | |
| cg25024074 + | 96 | 87 | 64 | 100 |
| cg01588438 + | | | | |
| cg23977631 | | | | |
| cg06952671 + | 97 | 85 | 64 | 100 |
| cg01988129 + | | | | |
| cg23977631 | | | | |
| cg21995919 + | 97 | 82 | 60 | 100 |
| cg01988129 + | | | | |
| cg23977631 | | | | |
| cg25024074 + | 97 | 88 | 64 | 100 |
| cg01988129 + | | | | |
| cg23977631 | | | | |
| cg06952671 + | 96 | 85 | 63 | 100 |
| cg20295442 + | | | | |
| cg23977631 | | | | |
| cg21995919 + | 96 | 81 | 59 | 100 |
| cg20295442 + | | | | |
| cg23977631 | | | | |
| cg25024074 + | 96 | 87 | 63 | 100 |
| cg20295442 + | | | | |
| cg23977631 | | | | |

### Example 4: DNA Isolation, Bisulfite Conversion, and Promoter CpG Island Methylation Analyses on Blood Samples

DNA methylation analysis was performed on 23 blood samples using the MOB (methylation on bead) technique as described by Keeley et al. (2013) [13].

### REFERENCES

1. Imperiale, T. F. et al., N. Engl. J. Med. 370; 1287-97 (2014).
2. Suzuki & Bird, Nature Reviews Genetics 9, 465-476 (2008).
3. Phillips T., Nature education 1(1): 116 (2008).
4. Moran, S., et al., Epigenomics 3; 389-399 (2016).
5. Gao L, van den Hurk K, Moerkerk P T, et al. Promoter CpG Island Hypermethylation in Dysplastic Nevus and Melanoma: CLDN11 as an Epigenetic Biomarker for Malignancy. J Invest Dermatol, 134:2957-66 (2014).
6. Pisanic, T. R. et al., Nucl. Acids Res. 43: e154 (2015).
7. Melotte et al., Cancer Prev. Res. 8: 157-164 (2015)].
8. Hellebrekers et al. Clin Cancer Res. 2009; 15: 3990-3997 (PMID: 19509152).
9. Melotte et al., J. Natl. Cancer Inst. 101: 916-927 (2009).
10. Glockner et al. Cancer Res. 69: 4691-4699 (2009) (PMID: 19435926).
11. Mant, D. et al., Br. J. Gen. Pract. 40: 423-425 (1990).
12. Draht, M. X. G. Prognostic DNA methylation markers for sporadic colorectal cancer: A sytematic review. Clinical Epigenetics 10, (2018).

1. A method of detecting alterations in expression status in a sample containing colorectal tumor or colorectal cancer nucleic acid taken from a human subject, the method comprising:
performing an assay on the sample and detecting the expression status of at least one gene that differs from a reference expression status;
wherein the at least one gene is a gene selected from TM6SF1, FAM19A4, FBLIM1, SLC35F3, SLC6A2, TMEM130, PCDH7, VIPR2, AMPH, TRHDE, GALNT13, NALCN, KCNIP4, NLGN4X, LRRC7, FAM184B, SYT9, and MARCHF11.

2. The method of claim 1, wherein the at least one gene comprises TM6SF1.

3. The method according claim 2, comprising at least one additional gene selected from SLC27A6, FAM19A4, FBLIM1, SLC35F3, SLC6A2, TMEM130, ADHFE1, PCDH7, VIPR2, AMPH, TRHDE, GALNT13, NALCN, KCNIP4, NLGN4X, LRRC7, FAM184B, SYT9, MARCHF11, NDRG4, LONRF2, ITGA4, UNC5C, GFRA1, SORCS1, SNAP91, HAND2, and GDNF.

4. A method for detecting a predisposition to, or the presence of colorectal cancer comprising:
detecting alterations in expression status in a sample according to the method of claim 1,
wherein the detection of an altered expression is indicative of a predisposition to, or the presence of colorectal cancer.

5. The method according to claim 1, wherein expression status of at least two, three, four, five, six, seven, eight, nine or ten genes are detected.

6. (canceled)

7. (canceled)

8. The method according to claim 1, wherein the alteration in expression status is detected by detecting a methylation status of the at least one gene or a methylation status of a promoter of the at least one gene.

9. The method according to claim 8, wherein hypermethylation of the at least one gene or promoter of the at least one gene is indicative of colorectal cancer.

10. The method according to claim 8, wherein hypomethylation of the at least one gene or promoter of the at least one gene is indicative of a predisposition to, or the presence of colorectal cancer.

11. The method according to claim 9, wherein hypermethylation of the promoter of the at least one gene comprises hypermethylation of the promoter of TM6SF1 and hypermethylation of a promoter of a gene selected from the group consisting of SLC27A6, FAM19A4, FBLIM1, SLC35F3, SLC6A2, TMEM130, ADHFE1, PCDH7, VIPR2, AMPH, TRHDE, GALNT13, NALCN, KCNIP4, NLGN4X, LRRC7, FAM184B, SYT9, MARCHF11, NDRG4, LONRF2, ITGA4, UNC5C, GFRA1, SORCS1, SNAP91, HAND2, and GDNF and is indicative of a predisposition to, or the presence of colorectal cancer.

12. (canceled)

13. The method according to claim 8, wherein the at least one gene is TM6SF1 and the promoter of the at least one gene comprises SEQ ID NO: 63 or SEQ ID NO: 41.

14. The method according to claim 8, wherein hypomethylation is determined by comparing the methylation status of the at least one gene or the methylation status of the promoter of the at least one gene to a reference methylation status, wherein the reference methylation status is from a subject not having CRC, optionally a subject that is negative for any colon abnormality according to colonoscopy.

15. The method according to claim 1, wherein the sample is selected from the group consisting of stool, blood, urine, serum, plasma, biopsies, and rectal swabs.

16. The method according to claim 8, wherein the at least one gene is TM6SF1 and the methylation status of the promoter of TM6SF1 is determined by analyzing a methylation level of at least one CpG dinucleotide in the promoter of TM6SF1.

17. The method according to claim 8, wherein the methylation status of the promoter of TM6SF1 and the methylation status of at least one promoter of a gene selected from SLC27A6, FAM19A4, FBLIM1, SLC35F3, SLC6A2, TMEM130, ADHFE1, PCDH7, VIPR2, AMPH, TRHDE, GALNT13, NALCN, KCNIP4, NLGN4X, LRRC7, FAM184B, SYT9, MARCHF11, NDRG4, LONRF2, ITGA4, UNC5C, GFRA1, SORCS1, SNAP91, HAND2, and GDNF is determined by analyzing the methylation level of at least one CpG dinucleotide in each of the promoters of the gene selected.

18. (canceled)

19. The method according to claim 8, wherein the at least one gene comprises TM6SF1 and measuring a methylation status comprises using a probe according to SEQ ID NO: 129 and primers according to SEQ ID NO: 85 and SEQ ID NO: 107 to measure the methylation status of the promoter of the gene TM6SF1.

20. The method according to claim 1, further comprising subjecting the subject to a colon biopsy.

21. The method accordingly to claim 1, further comprising administering to the subject a treatment method for colorectal cancer, optionally selected from a chemotherapy, a biological agent, a surgery, and/or radiation therapy.

22. A kit for detecting alterations in expression status in a nucleic acid containing colorectal tumor or colorectal cancer sample, the kit comprising:
labelled oligonucleotides to specifically detect methylation levels or promoter methylation levels of at least one gene selected from TM6SF1, SLC27A6, FAM19A4, FBLIM1, SLC35F3, SLC6A2, TMEM130, PCDH7, VIPR2, AMPH, TRHDE, GALNT13, NALCN, KCNIP4, NLGN4X, LRRC7, FAM184B, SYT9, MARCHF11, ITGA4, UNC5C, GFRA1, SORCS1, SNAP91, HAND2, GDNF, LONRF2, NDRG4 and ADHFE1.

23. The kit of claim 24, wherein the labelled oligonucleotides specifically hybridize to the promoter of gene TM6SF1.

24. A system for detecting alterations in expression status in a nucleic acid containing colorectal tumor or colorectal cancer sample, the system comprising:
labelled oligonucleotides configured to specifically detect methylation levels or promoter methylation levels of at least one gene selected from TM6SF1, SLC27A6, FAM19A4, FBLIM1, SLC35F3, SLC6A2, TMEM130, PCDH7, VIPR2, AMPH, TRHDE, GALNT13, NALCN, KCNIP4, NLGN4X, LRRC7, FAM184B, SYT9, MARCHF11, ITGA4, UNC5C, GFRA1, SORCS1, SNAP91, HAND2, GDNF, LONRF2, NDRG4 and ADHFE1.

25.-26. (canceled)
